SEQUENCE LISTING (118 sequences)

SEQ ID NO: 1 - protein, 58 aa, unidentified
XXXXCXXXXXXGXCXXXXXXXXXXXXXXXCXXFXXXGCXXXXXXXXXXXXCXXXCXXX

SEQ ID NO: 2 - protein, 58 aa, synthetic construct
KPDFCFLEEDPGPCRAVKRRYFYNNQTKQCERFKYGGCLGNMNNFETLEECKNICEDG

SEQ ID NO: 3 - protein, 58 aa, synthetic construct
KPDFCFLENDPGPCRALKRRYFYNNQTKQCERFYYGGCFGNMNNFVTLEECKNICEDG

SEQ ID NO: 4 - protein, 58 aa, synthetic construct
KPDFCFLENDPGWCRALKRRYFYNNQTKQCERFGYGGCLGNMNNFVTLEECKNICEDG

SEQ ID NO: 5 - protein, 58 aa, synthetic construct
KPDFCFLEEDPGPCRALKRRYFYNNQTKQCERFYYGGCFGNMNNFVTLEECKNICEDG

SEQ ID NO: 6 - protein, 58 aa, synthetic construct
KPDFCFLENDPGPCRAAKRRYFYNNQTKQCERFYYGGCLGNMNNFVTLEECKNICEDG

SEQ ID NO: 7 - protein, 58 aa, synthetic construct
KPDFCFLENDPGPCRALKRRYFYNNQTKQCERFYYGGCLGNMNNFVTLEECKNICEDG

SEQ ID NO: 8 - protein, 58 aa, synthetic construct
KPDFCFLENDPGPCRALKLRYFYNNQTKQCERFGYGGCLGNMNNFVTLEECKNICEDG

SEQ ID NO: 9 - protein, 58 aa, synthetic construct
KPDFCFLEEDPGPCRALKRRYFYNNQTKQCERFGYGGCLGNMNNFVTLEECKNICEDG

SEQ ID NO: 10 - protein, 58 aa, synthetic construct
KPDFCFLEEDPGPCRAAKRRYFYNNQTKQCERFYYGGCFGNMNNFVTLEECKNICEDG

SEQ ID NO: 11 - protein, 58 aa, synthetic construct
KPDFCFLENDPGPCRALKRRYFYNNQTKQCERFYYGGCFGNMNNFETLEECKNICEDG

SEQ ID NO: 12 - protein, 58 aa, synthetic construct
KPDFCFLENDPGPCRALKRRYFYNNQTKQCERFGYGGCLGNMNNFVTLEECKNICEDG

SEQ ID NO: 13 - protein, 58 aa, synthetic construct
KPDFCFLEEDPGPCRALKRRYFYNNQTKQCERFGYGGCFGNMNNFVTLEECKNICEDG

SEQ ID NO: 14 - protein, 58 aa, synthetic construct
KPDFCFLENDPGLCRALKRRYFYNNQTKQCERFGYGGCLGNMNNFVTLEECKNICEDG

SEQ ID NO: 15 - protein, 58 aa, synthetic construct
KPDFCFLENDPGPCRALKRRYFYNNQTKQCERFYYGGCLGNMNNFETLEECKNICEDG

SEQ ID NO: 16 - protein, 58 aa, synthetic construct
KPDFCFLEEDPGPCRALKRRYFYNNQTKQCERFSYGGCFGNMNNFVTLEECKNICEDG

SEQ ID NO: 17 - protein, 58 aa, synthetic construct
KPDFCFLEEDPGPCRALKLRYFYNNQTKQCERFGYGGCFGNMNNFVTLEECKNICEDG

SEQ ID NO: 18 - protein, 58 aa, synthetic construct
KPDFCFLENDPGLCRAAAVRYFYNNQTKQCERFYYGGCLGNMNNFVTLEECKNICEDG

SEQ ID NO: 19 - protein, 58 aa, synthetic construct
KPDFCFLENDPGPCRAAAVRYFYNNQTKQCERFYYGGCLGNMNNFVTLEECKNICEDG

SEQ ID NO: 20 - protein, 58 aa, synthetic construct
KPDFCFLEEDPGWCRALKRRYFYNNQTKQCERFGYGGCFGNMNNFVTLEECKNICEDG

SEQ ID NO: 21 - protein, 58 aa, synthetic construct
KPDFCFLENDPGVCRAAAVRYFYNNQTKQCERFYYGGCLGNMNNFVTLEECKNICEDG

SEQ ID NO: 22 - protein, 58 aa, synthetic construct
KPDFCFLENDPGPCRALARRYFYNNQTKQCERFYYGGCLGNMNNFVTLEECKNICEDG

SEQ ID NO: 23 - skipped

SEQ ID NO: 24 - protein, 58 aa, synthetic construct
KPDFCFLENDPGPCRAVKRRYFYNNQTKQCERFGYGGCLGNMNNFVTLEECKNICEDG

SEQ ID NO: 25 - protein, 58 aa, synthetic construct
KPDFCFLEEDPGPCRAVKRRYFYNNQTKQCERFYYGGCLGNMNNFETLEECKNICEDG

SEQ ID NO: 26 - protein, 58 aa, synthetic construct
KPDFCFLENDPGWCRAAKRRYFYNNQTKQCERFGYGGCLGNMNNFVTLEECKNICEDG

SEQ ID NO: 27 - protein, 58 aa, synthetic construct
KPDFCFLENDPGPCRAAKRRYFYNNQTKQCERFGYGGCLGNMNNFVTLEECKNICEDG

SEQ ID NO: 28 - protein, 58 aa, synthetic construct
KPDFCFLENDPGVCRALAVRYFYNNQTKQCERFYYGGCLGNMNNFVTLEECKNICEDG

SEQ ID NO: 29 - protein, 58 aa, synthetic construct
KPDFCFLENDPGWCRAAKLRYFYNNQTKQCERFGYGGCLGNMNNFVTLEECKNICEDG

SEQ ID NO: 30 - protein, 58 aa, synthetic construct
KPDFCFLEEDPGPCRALKRRYFYNNQTKQCERFKYGGCLGNMNNFETLEECKNICEDG

SEQ ID NO: 31 - protein, 58 aa, synthetic construct
KPDFCFLEEDPGPCRAVKRRYFYNNQTKQCERFGYGGCLGNMNNFETLEECKNICEDG

SEQ ID NO: 32 - protein, 58 aa, synthetic construct
KPDFCFLENDPGPCRAVKRRYFYNNQTKQCERFKYGGCLGNMNNFETLEECKNICEDG

SEQ ID NO: 33 - protein, 58 aa, synthetic construct
XXXXCXXXNXXGPCRALKRXXXXXXXXXXCXXFYXXGCFXXXXXXVXXXXCXXXCXXX

SEQ ID NO: 34 - protein, 58 aa, synthetic construct
XXXXCXXXNXXGWCRALKRXXXXXXXXXXCXXFGXXGCLXXXXXXVXXXXCXXXCXXX

SEQ ID NO: 35 - protein, 58 aa, synthetic construct
XXXXCXXXEXXGPCRALKRXXXXXXXXXXCXXFYXXGCFXXXXXXVXXXXCXXXCXXX

SEQ ID NO: 36 - protein, 58 aa, synthetic construct
XXXXCXXXNXXGPCRAAKRXXXXXXXXXXCXXFYXXGCLXXXXXXVXXXXCXXXCXXX

SEQ ID NO: 37 - protein, 58 aa, synthetic construct
XXXXCXXXNXXGPCRALKRXXXXXXXXXXCXXFYXXGCLXXXXXXVXXXXCXXXCXXX

SEQ ID NO: 38 - protein, 58 aa, synthetic construct
XXXXCXXXNXXGPCRALKLXXXXXXXXXXCXXFGXXGCLXXXXXXVXXXXCXXXCXXX

SEQ ID NO: 39 - protein, 58 aa, synthetic construct
XXXXCXXXEXXGPCRALKRXXXXXXXXXXCXXFGXXGCLXXXXXXVXXXXCXXXCXXX

SEQ ID NO: 40 - protein, 58 aa, synthetic construct
XXXXCXXXEXXGPCRAAKRXXXXXXXXXXCXXFYXXGCFXXXXXXVXXXXCXXXCXXX

SEQ ID NO: 41 - protein, 58 aa, synthetic construct
XXXXCXXXNXXGPCRALKRXXXXXXXXXXCXXFYXXGCFXXXXXXEXXXXCXXXCXXX

SEQ ID NO: 42 - protein, 58 aa, synthetic construct
XXXXCXXXNXXGPCRALKRXXXXXXXXXXCXXFGXXGCLXXXXXXVXXXXCXXXCXXX

SEQ ID NO: 43 - protein, 58 aa, synthetic construct
XXXXCXXXEXXGPCRALKRXXXXXXXXXXCXXFGXXGCFXXXXXXVXXXXCXXXCXXX

SEQ ID NO: 44 - protein, 58 aa, synthetic construct
XXXXCXXXNXXGLCRALKRXXXXXXXXXXCXXFGXXGCLXXXXXXVXXXXCXXXCXXX

SEQ ID NO: 45 - protein, 58 aa, synthetic construct
XXXXCXXXNXXGPCRALKRXXXXXXXXXXCXXFYXXGCLXXXXXXEXXXXCXXXCXXX

SEQ ID NO: 46 - protein, 58 aa, synthetic construct
XXXXCXXXEXXGPCRALKRXXXXXXXXXXCXXFSXXGCFXXXXXXVXXXXCXXXCXXX

SEQ ID NO: 47 - protein, 58 aa, synthetic construct
XXXXCXXXEXXGPCRALKLXXXXXXXXXXCXXFGXXGCFXXXXXXVXXXXCXXXCXXX

SEQ ID NO: 48 - protein, 58 aa, synthetic construct
XXXXCXXXNXXGLCRAAAVXXXXXXXXXXCXXFYXXGCLXXXXXXVXXXXCXXXCXXX

SEQ ID NO: 49 - protein, 58 aa, synthetic construct
XXXXCXXXNXXGPCRAAAVXXXXXXXXXXCXXFYXXGCLXXXXXXVXXXXCXXXCXXX

SEQ ID NO: 50 - protein, 58 aa, synthetic construct
XXXXCXXXEXXGWCRALKRXXXXXXXXXXCXXFGXXGCFXXXXXXVXXXXCXXXCXXX

SEQ ID NO: 51 - protein, 58 aa, synthetic construct
XXXXCXXXNXXGVCRAAAVXXXXXXXXXXCXXFYXXGCLXXXXXXVXXXXCXXXCXXX

SEQ ID NO: 52 - protein, 58 aa, synthetic construct
XXXXCXXXNXXGPCRALARXXXXXXXXXXCXXFYXXGCLXXXXXXVXXXXCXXXCXXX

SEQ ID NO: 53 - skipped

SEQ ID NO: 54 - protein, 58 aa, synthetic construct
XXXXCXXXNXXGPCRAVKRXXXXXXXXXXCXXFGXXGCLXXXXXXVXXXXCXXXCXXX

SEQ ID NO: 55 - protein, 58 aa, synthetic construct
XXXXCXXXEXXGPCRAVKRXXXXXXXXXXCXXFYXXGCLXXXXXXEXXXXCXXXCXXX

SEQ ID NO: 56 - protein, 58 aa, synthetic construct
XXXXCXXXNXXGWCRAAKRXXXXXXXXXXCXXFGXXGCLXXXXXXVXXXXCXXXCXXX

SEQ ID NO: 57 - DNA, 227 bp, synthetic construct
  source 1..227: mol_type = other DNA
gggtccggaggctcgggcaaaccggacttctgcttcctggaataataaccgggttaatgccgtgcgtaaaaacgtcgttacttctacaacaaccagaccaaacagtgcgaacgtttctaatacggtggttgctaaggtaacatgaacaacttctaaaccctggaagaatgcaaaaacatctgcgaagacggtggcggcagcggcggcagcggcgggagctccagcgc

SEQ ID NO: 58 - DNA, 85 bp, synthetic construct
  source 1..85: mol_type = other DNA
cgggcaaaccggacttctgcttcctggaannknnkccgggtnnktgccgtgcgstgaaacgtcgttacttctacaacaaccagac

SEQ ID NO: 59 - DNA, 86 bp, synthetic construct
  source 1..86: mol_type = other DNA
caaacagtgcgaacgtttcnnktacggtggttgcnnkggtaacatgaacaacttcnnkaccctggaagaatgcaaaaacatctgcg

SEQ ID NO: 60 - DNA, 227 bp, synthetic construct
  source 1..227: mol_type = other DNA
gggtccggaggctcgggcaaaccggacttctgcttcctggaaaacgacccgggttaatgctaataataataataacgttacttctacaacaaccagaccaaacagtgcgaacgtttctattacggtggttgcctgggtaacatgaacaacttcgtgaccctggaagaatgcaaaaacatctgcgaagacggtggcggcagcggcggcagcggcgggagctccagcgc

SEQ ID NO: 61 - DNA, 64 bp, synthetic construct
  source 1..64: mol_type = other DNA
cctggaaaacgacccgggtnnktgcnnknnknnknnknnkcgttacttctacaacaaccagacc

SEQ ID NO: 62 - DNA, 85 bp, synthetic construct
  source 1..85: mol_type = other DNA
cgggcaaaccggacttctgcttcctggaagaagacccgggttaatgctaagcgtaaaaacttcgttacttctacaacaaccagac

SEQ ID NO: 63 - DNA, 86 bp, synthetic construct
  source 1..86: mol_type = other DNA
caaacagtgcgaacgtttctaatacggtggttgcttcggtaacatgaacaacttcgtaaccctggaagaatgcaaaaacatctgcg

SEQ ID NO: 64 - DNA, 227 bp, synthetic construct
  source 1..227: mol_type = other DNA
gggtccggaggctcgggcaaaccggacttctgcttcctggaagaagacccgggttaatgctaagcgtaaaaacgtcgttacttctacaacaaccagaccaaacagtgcgaacgtttctaatacggtggttgcttcggtaacatgaacaacttcgtaaccctggaagaatgcaaaaacatctgcgaagacggtggcggcagcggcggcagcggcgggagctccagcgc

SEQ ID NO: 65 - DNA, 85 bp, synthetic construct
  source 1..85: mol_type = other DNA
cgggcaaaccggacttctgcttcctggaagaagacccgggtnnktgcavagcgnnkaaacttcgttacttctacaacaaccagac

SEQ ID NO: 66 - DNA, 65 bp, synthetic construct
  source 1..65: mol_type = other DNA
caaacagtgcgaacgtttcnnktacggtggttgcttcggtaacatgaacaacttcgtaaccctgg

SEQ ID NO: 67 - DNA, 61 bp, synthetic construct
  source 1..61: mol_type = other DNA
gaagtaacgacgtttcagcgcacggcacggacccgggtcgttttccaggaagcagaagtcc

SEQ ID NO: 68 - DNA, 23 bp, synthetic construct
  source 1..23: mol_type = other DNA
ctccatcgccatgatgtctaacg

SEQ ID NO: 69 - DNA, 22 bp, synthetic construct
  source 1..22: mol_type = other DNA
gctagttattgctcagcggtgg

SEQ ID NO: 70 - DNA, 69 bp, synthetic construct
  source 1..69: mol_type = other DNA
cagtgcgaacgtttcggatacggtggttgcctgggtaacatgaacaacttcgtaaccctggaagaatgc

SEQ ID NO: 71 - DNA, 69 bp, synthetic construct
  source 1..69: mol_type = other DNA
cagtgcgaacgtttctattacggtggttgcctgggtaacatgaacaacttcgtaaccctggaagaatgc

SEQ ID NO: 72 - DNA, 69 bp, synthetic construct
  source 1..69: mol_type = other DNA
cagtgcgaacgtttctattacggtggttgctttggtaacatgaacaacttcgtaaccctggaagaatgc

SEQ ID NO: 73 - DNA, 69 bp, synthetic construct
  source 1..69: mol_type = other DNA
cagtgcgaacgtttctattacggtggttgcctgggtaacatgaacaacttcgaaaccctggaagaatgc

SEQ ID NO: 74 - DNA, 69 bp, synthetic construct
  source 1..69: mol_type = other DNA
cagtgcgaacgtttctattacggtggttgctttggtaacatgaacaacttcgaaaccctggaagaatgc

SEQ ID NO: 75 - DNA, 56 bp, synthetic construct
  source 1..56: mol_type = other DNA
ggttgttgtagaagtaacgaactgccgccgcacggcacacacccgggtcgttttcc

SEQ ID NO: 76 - DNA, 56 bp, synthetic construct
  source 1..56: mol_type = other DNA
ggttgttgtagaagtaacgaactgccgccgcacggcacagacccgggtcgttttcc

SEQ ID NO: 77 - DNA, 56 bp, synthetic construct
  source 1..56: mol_type = other DNA
ggttgttgtagaagtaacgaactgccgccgcacggcacggacccgggtcgttttcc

SEQ ID NO: 78 - DNA, 56 bp, synthetic construct
  source 1..56: mol_type = other DNA
ggttgttgtagaagtaacgaactgccagcgcacggcacacacccgggtcgttttcc

SEQ ID NO: 79 - DNA, 35 bp, synthetic construct
  source 1..35: mol_type = other DNA
gacttctgcttcctggaaaatgacccgggtccgtg

SEQ ID NO: 80 - DNA, 35 bp, synthetic construct
  source 1..35: mol_type = other DNA
cacggacccgggtcattttccaggaagcagaagtc

SEQ ID NO: 81 - DNA, 33 bp, synthetic construct
  source 1..33: mol_type = other DNA
cagtgcgaacgtttcggatacggtggttgcctg

SEQ ID NO: 82 - DNA, 33 bp, synthetic construct
  source 1..33: mol_type = other DNA
caggcaaccaccgtatccgaaacgttcgcactg

SEQ ID NO: 83 - DNA, 33 bp, synthetic construct
  source 1..33: mol_type = other DNA
cagtgcgaacgtttctattacggtggttgcctg

SEQ ID NO: 84 - DNA, 33 bp, synthetic construct
  source 1..33: mol_type = other DNA
caggcaaccaccgtaatagaaacgttcgcactg

SEQ ID NO: 85 - DNA, 33 bp, synthetic construct
  source 1..33: mol_type = other DNA
ccgtgccgtgcgctgaaacgtcgttacttctac

SEQ ID NO: 86 - DNA, 33 bp, synthetic construct
  source 1..33: mol_type = other DNA
gtagaagtaacgacgtttcagcgcacggcacgg

SEQ ID NO: 87 - DNA, 33 bp, synthetic construct
  source 1..33: mol_type = other DNA
ccgtgccgtgcggtgaaacgtcgttacttctac

SEQ ID NO: 88 - DNA, 33 bp, synthetic construct
  source 1..33: mol_type = other DNA
gtagaagtaacgacgtttcaccgcacggcacgg

SEQ ID NO: 89 - DNA, 34 bp, synthetic construct
  source 1..34: mol_type = other DNA
gaaaacgacccgggttggtgccgtgcgctgaaac

SEQ ID NO: 90 - DNA, 34 bp, synthetic construct
  source 1..34: mol_type = other DNA
gtttcagcgcacggcaccaacccgggtcgttttc

SEQ ID NO: 91 - DNA, 34 bp, synthetic construct
  source 1..34: mol_type = other DNA
gaaaacgacccgggtctgtgccgtgcgctgaaac

SEQ ID NO: 92 - DNA, 34 bp, synthetic construct
  source 1..34: mol_type = other DNA
gtttcagcgcacggcacagacccgggtcgttttc

SEQ ID NO: 93 - DNA, 33 bp, synthetic construct
  source 1..33: mol_type = other DNA
ccgtgccgtgcggcgaaacgtcgttacttctac

SEQ ID NO: 94 - DNA, 33 bp, synthetic construct
  source 1..33: mol_type = other DNA
gtagaagtaacgacgtttcgccgcacggcacgg

SEQ ID NO: 95 - DNA, 35 bp, synthetic construct
  source 1..35: mol_type = other DNA
gccgtgcgctgaaacttcgttacttctacaacaac

SEQ ID NO: 96 - DNA, 35 bp, synthetic construct
  source 1..35: mol_type = other DNA
gttgttgtagaagtaacgaagtttcagcgcacggc

SEQ ID NO: 97 - DNA, 48 bp, synthetic construct
  source 1..48: mol_type = other DNA
gaaaacgacccgggttggtgccgtgcggcgaaacgtcgttacttctac

SEQ ID NO: 98 - DNA, 48 bp, synthetic construct
  source 1..48: mol_type = other DNA
gtagaagtaacgacgtttcgccgcacggcaccaacccgggtcgttttc

SEQ ID NO: 99 - DNA, 54 bp, synthetic construct
  source 1..54: mol_type = other DNA
gaaaacgacccgggttggtgccgtgcggcgaaacttcgttacttctacaacaac

SEQ ID NO: 100 - DNA, 54 bp, synthetic construct
  source 1..54: mol_type = other DNA
gttgttgtagaagtaacgaagtttcgccgcacggcaccaacccgggtcgttttc

SEQ ID NO: 101 - DNA, 33 bp, synthetic construct; identical to SEQ ID NO: 93
  source 1..33: mol_type = other DNA

SEQ ID NO: 102 - DNA, 33 bp, synthetic construct; identical to SEQ ID NO: 94
  source 1..33: mol_type = other DNA

SEQ ID NO: 103 - DNA, 32 bp, synthetic construct
  source 1..32: mol_type = other DNA
gccgtgcgctggcacgtcgttacttctacaac

SEQ ID NO: 104 - DNA, 32 bp, synthetic construct
  source 1..32: mol_type = other DNA
gttgtagaagtaacgacgtgccagcgcacggc

SEQ ID NO: 105-106 - skipped (2 entries)

SEQ ID NO: 107 - DNA, 200 bp, synthetic construct
  source 1..200: mol_type = other DNA
ggatccaaaccggacttctgcttcctggaagaagacccgggtccgtgccgtgcgctgaaacgtcgttacttctacaacaaccagaccaaacagtgcgaacgtttcggatacggtggttgcctgggtaacatgaacaacttcgtaaccctggaagaatgcaaaaacatctgcgaagacggttaataagcttggcactcgag

SEQ ID NO: 108 - DNA, 200 bp, synthetic construct
  source 1..200: mol_type = other DNA
ggatccaaaccggacttctgcttcctggaagaagacccgggtccgtgccgtgcgctgaaacgtcgttacttctacaacaaccagaccaaacagtgcgaacgtttcggatacggtggttgctttggtaacatgaacaacttcgtaaccctggaagaatgcaaaaacatctgcgaagacggttaataagcttggcactcgag

SEQ ID NO: 109 - DNA, 200 bp, synthetic construct
  source 1..200: mol_type = other DNA
ggatccaaaccggacttctgcttcctggaagaagacccgggtccgtgccgtgcgctgaaacgtcgttacttctacaacaaccagaccaaacagtgcgaacgtttcagctacggtggttgctttggtaacatgaacaacttcgtaaccctggaagaatgcaaaaacatctgcgaagacggttaataagcttggcactcgag

SEQ ID NO: 110 - DNA, 200 bp, synthetic construct
  source 1..200: mol_type = other DNA
ggatccaaaccggacttctgcttcctggaagaagacccgggttggtgccgtgcgctgaaacgtcgttacttctacaacaaccagaccaaacagtgcgaacgtttcggatacggtggttgctttggtaacatgaacaacttcgtaaccctggaagaatgcaaaaacatctgcgaagacggttaataagcttggcactcgag

SEQ ID NO: 111 - DNA, 200 bp, synthetic construct
  source 1..200: mol_type = other DNA
ggatccaaaccggacttctgcttcctggaagaagacccgggtccgtgccgtgcgctgaaactgcgttacttctacaacaaccagaccaaacagtgcgaacgtttcggatacggtggttgctttggtaacatgaacaacttcgtaaccctggaagaatgcaaaaacatctgcgaagacggttaataagcttggcactcgag

SEQ ID NO: 112 - DNA, 200 bp, synthetic construct
  source 1..200: mol_type = other DNA
ggatccaaaccggacttctgcttcctggaagaagacccgggtccgtgccgtgcggcgaaacgtcgttacttctacaacaaccagaccaaacagtgcgaacgtttctattacggtggttgctttggtaacatgaacaacttcgtaaccctggaagaatgcaaaaacatctgcgaagacggttaataagcttggcactcgag

SEQ ID NO: 113 - DNA, 200 bp, synthetic construct
  source 1..200: mol_type = other DNA
ggatccaaaccggacttctgcttcctggaagaagacccgggtccgtgccgtgcgctgaaacgtcgttacttctacaacaaccagaccaaacagtgcgaacgtttctattacggtggttgctttggtaacatgaacaacttcgtaaccctggaagaatgcaaaaacatctgcgaagacggttaataagcttggcactcgag

SEQ ID NO: 114 - protein, 299 aa, synthetic construct
GSKPDFCFLENDPGPCRALKRRYFYNNQTKQCERFYYGGCLGNMNNFETLEECKNICEDGGTGGGSGSSSGSDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 58 aa, synthetic construct; identical to SEQ ID NO: 1

SEQ ID NO: 116 - protein, 58 aa, Homo sapiens
KPDFCFLEEDPGICRGYITRYFYNNQTKQCERFKYGGCLGNMNNFETLEECKNICEDG

SEQ ID NO: 117 - DNA, 550 bp, synthetic construct
  source 1..550: mol_type = other DNA
catatgaaaataaaaacaggtgcacgcatcctcgcattatccgcattaacgacgatgatgttttccgcctcggcgctagcatcggattataaagacgatgatgacaaagcttccggcgggtccggaggctcgggcaaaccggacttctgcttcctggaagaagacccgggtatctgccgtggttacatcacccgttacttctacaacaaccagaccaaacagtgcgaacgtttcaaatacggtggttgcctgggtaacatgaacaacttcgaaaccctggaagaatgcaaaaacatctgcgaagacggtggcggcagcggcggcagcggcgggagctccagcgccgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctaataatctagagcggccgccccgcggtggagcttgaattcgccctatagtgagtcgt

SEQ ID NO: 118 - protein, 163 aa, synthetic construct
HMKIKTGARILALSALTTMMFSASALASDYKDDDDKASGGSGGSGKPDFCFLEEDPGICRGYITRYFYNNQTKQERFKYGGCLGNMNNFETLEECKNICEDGGGSGGSGGSSSAEGDDPAKAAFNSLQASATEYIGYAWAMVVVIVGATIGIKLFKKFTSKAS

(19) United States
(12) Patent Application Publication
(10) Pub. No.: US 2026/0098080 A1
(43) Pub. Date: Apr. 9, 2026

(54) A MODIFIED PROTEIN SCAFFOLD AND USE THEREOF

(71) Applicant: EVOLVERITAS BIOTECHNOLÓGIAI KORLÁTOLT FELELOSSÉGU TÁRSASÁG, Szentes (HU)

(72) Inventors: Péter GÁL, Budapest (HU); Gergo Zsolt GÓGL, Pilisborosjeno (HU); Bence KISS, Budapest (HU); Zsombor KÖLLER, Budapest (HU); Zoltán Attila NAGY, Budapest (HU); Zoltán Bálint NÉMETH, Budapest (HU); Gábor PÁL, Budapest (HU); Andrea PÁRISNÉ DR KOCSIS, Budapest (HU)

(73) Assignee: EVOLVERITAS BIOTECHNOLÓGIAI KORLÁTOLT FELELOSSÉGU TÁRSASÁG, Szentes (HU)

(21) Appl. No.: 19/114,995

(22) PCT Filed: Sep. 29, 2023
(86) PCT No.: PCT/HU2023/050068

§ 371 Date: Mar. 25, 2025

(30) Foreign Application Priority Data
Sep. 29, 2022 (HU) P2200390

(51) Int. Cl. C07K 14/81; A61K 38/00; G01N 33/573

(52) U.S. Cl. CPC C07K 14/8114; C12Y 304/21104; G01N 33/573; A61K 38/00; G01N 2333/96433; G01N 2500/04

(57) ABSTRACT

A protein comprising amino acid sequence of SEQ ID NO: 115, within which a segment of general formula Ih-mod GX₁CX_{1V}X₂X₃X₄X₅ is present
where
X₁ is any of F, Y, L, P, Q, M, V, W, A, or T,
X_{1V} is R, or K,
X₂ is any of A, G, S, or T,
X₃ is any amino acid of the 17-set, where the 17-set comprises A, I, L, F, or Y,
X₄ is any of K, I, Q, R, H, S, F, M, N, L, or V, and
X₅ is any of R, V, I, K, M, Q, E, F, L, N, Y, D, S, H;
and
b) in position 34 of SEQ ID NO: 115 it contains an amino acid selected from the 34-set contains any amino acid of the 34-set, where the 34-set comprises Y, I, F, G, V and S, and use in pharmaceutical preparations, kits, screening procedures.

### CROSS REFERENCE TO RELATED APPLICATIONS

This application is a 35 U.S.C. § 371 National Stage of International Patent Application No. PCT/HU2023/050068, filed Sep. 29, 2023, claiming benefit from Hungairan Patent Application No. P2200390, filed Sep. 29, 2022, the disclosures of which are incorporated herein in their entirety by reference, and priority is claimed to each of the foregoing.

### REFERENCE TO AN ELECTRONIC SEQUENCE LISTING

The contents of the electronic sequence listing named EVO2 MASP-2 inhibitors.xml, Size: 107,814 bytes; and Date of Creation: Sep. 29, 2022) is herein incorporated by reference in its entirety.

### FIELD OF THE INVENTION

The present invention relates to a family of novel protein scaffolds, to a process for the production of proteins having such scaffolds, and to the use of such proteins in the production of medicines.

### BACKGROUND OF THE INVENTION

The complement system is a highly conserved part of the innate immune system of the vertebrates including humans. The complement system is a network of different proteins including serine proteases, soluble and membrane-bound receptors and regulators. It is evolutionary and functionally closely related to other protein networks in the blood such as the blood coagulation and the fibrinolysis. As a part of the immune system, the complement system can recognize, label and eliminate invading pathogen microorganisms (viruses, bacteria, fungi) and dangerously altered self-structures such as virus infected cells, apoptotic/necrotic cells, cancer cells. The complement system forms one of the first lines of defense against the pathogenic microorganism. Besides being a major effector arm of the innate immune system, the complement system also connects the innate and adaptive immune response in many ways. Although it is a network of protein molecules it is capable of priming and modulating various cellular processes, as well (Merle 2015a; Merle 2015b; Hajishengallis 2017).

The complement system consists of more than 30 protein components. The main components are serine proteases which activate each-other in a cascade-like manner (Sim 2004). Activation involves limited proteolysis of the zymogen protease by the activated subcomponent. Other components include pattern recognition molecules, inhibitors, modulators, and cell-surface receptors. The major event during complement activation is the cleavage of the C3 component. C3 is cleaved into two pieces, C3a and C3b, by the C3 convertase enzyme complexes. As a result of a significant conformational change a thioester bond is exposed at the surface of the C3b molecule through which it can attach to the activation surface (e.g., bacterial cell, immune complexes) (Geisbrecht 2022). C3b is an opsonin that facilitates the clearance of the dangerous structures by the phagocytes, as well as it serves as a platform for further enhancement of complement activation and the initiation of the terminal pathway leading the destruction of the invaded cell. The C3a component is an anaphylatoxin which initiates inflammation through triggering immune cells activation (Ricklin 2016).

There are three routes through which C3 convertase complexes can be formed: the classical, the lectin and the alternative pathways. In the classical and the lectin pathways pattern recognition molecules bind to the activation surface and associated serine proteases initiate the proteolytic cascade. The pattern recognition molecule of the classical pathway is the C1q (Thielen 2017). C1q is composed of three kinds of polypeptide chains, A, B and C chains, and it has a structure resembling a bunch of six tulips. The A, B and C chains form a trimer which has a collagen-like arm at the N-terminal and a globular head at the C-terminal half of the molecule. Six of such trimers associate to form the entire C1q structure. In the blood circulation C1q is associated with two types of serine proteases: C1r and C1s. Two C1r and two C1s molecules form a tetramer (C1s-C1r-C1r-C1s) which binds to the collagenous arms of the C1q (Zwarthoff 2021). The resulting C1 complex is the initiation complex of the classical pathway. Upon activation, the globular heads of C1q bind to the activator structure. Typical activators of the classical pathway are immune complexes, C-reactive protein, and apoptotic cells (Diebolder 2014, Sharp 2019). In the C1 complex the C1r and C1s serine proteases are present in zymogenic form and become activated after the C1q binds to the activator surface. During activation, an Arg-Ile bond is cleaved in the activation peptide of the serine protease domain. The resulting two polypeptide chains are held together by a disulfide bond. The first enzymatic event in the classical pathway activation is the autoactivation of C1r. In the next step, active C1r cleaves and activates the zymogen C1s. Activated C1s is the executive protease of the C1 complex. It cleaves C4 and C2 components generating the classical pathway C3 convertase C4b2a (Gal 2009).

The same C3 convertase complex is generated through the lectin pathway. In the lectin pathway there are at least five different pattern recognition molecules: MBL (mannose-binding lectin), ficolin-1/-2/-3, CL-LK (Holmskov 2003). The global structure of MBL and ficolins resembles C1q, however, the trimeric subunits are composed of one kind of polypeptide chain. While C1q has a well-defined hexameric structure, the polymerization status of MBL and ficolins varies between dimer and hexamer. The most populated polymer form is the tetramer. The two collectins, collectin kidney 1 (CL-K1) and collectin liver 1 (CL-L1) form heterotrimeric subunits containing one CL-L1 and two CL-K1 polypeptide chains. There are three MBL-associated serine proteases (hereinafter referred to as MASPs): MASP-1, MASP-2 and MASP-3 (Dobó 2016a). MASP-1 and MASP-2 are present in the zymogen form and are associated with the pattern recognition molecules. MASP-1 and MASP-2 are the initiator proteases of the lectin pathway. MASP-1 and MASP-2 form homodimers via the N-terminal non-catalytic region. These homodimers bind to the pattern recognition molecules. A tetrameric pattern recognition molecule typically binds a single MASP dimer. Most of the activation complexes of the lectin pathway contain one kind of MASP. During lectin pathway activation the pattern recognition molecules bind to the activator surface (e.g., bacterial surface). MBL binds to the carbohydrate arrays on the surface of the bacteria in a Ca⁺ dependent manner. Ficolins bind acetylated compounds, typically to acetylated sugars (e.g., N-acetyl-glycosamine). On the surface MBL-MASP-1 and MBL-MASP-2 complexes are deposited next to each-other (Degn 2014). The first enzymatic event during lectin pathway activation is the autoactivation of MASP-1. Activated MASP-1 is the exclusive activator of MASP-2 (Héja 2012a). Only activated MASP-2 is capable of cleaving C4, while C2 is cleaved by both MASP-1 and MASP-2. The resulting C3 convertase complex (C4b2a) is identical to that of the classical pathway. The third complement pathway, the alternative pathway is closely connected to the lectin pathway. MASP-3, which is an alternative splice product of the MASP1 gene, is responsible for the activation of pro-Factor D (pro-FD) (Dobó 2016b). MASP-3 constitutively activates pro-FD even in the absence of any danger signal (PAMP or DAMP). Due to the constant MASP-3 proteolytic activity, FD is present in the activated form in the blood plasma. When C3b deposits on the activation surface it binds Factor B (FB), the serine protease component of the alternative pathway C3 convertase. FD has extremely narrow substrate specificity. C3-bound FB is the sole substrate of FD. After FD mediated cleavage of FB, the larger fragment Bb remains associated with C3b, and the smaller fragment (Ba) dissociates. The resulting alternative pathway convertase (C3bBb) cleaves other C3 molecules that could serve as platforms for more C3 convertase complexes. As a result of this, the alternative pathway forms a positive feed-back mechanism for enhancing complement activation regardless of the initiation pathway (Harboe 2004).

As the classical or the lectin pathway generates the first C3b molecules, the alternative pathway initiates and provides the amplification loop. The alternative pathway can also initiate on its own due to the so called “tick over” mechanism (Pangburn 1981). C3 hydrolyzes slowly in the fluid phase. The resulting C3 (H₂O) has a C3b-like conformation and binds FB. After FD mediated cleavage, the resulting C3(H₂O)Bb is a fluid-phase C3 convertase. This fluid-phase convertase continuously deposits C3b to the nearby surfaces. In the case of self-tissues membrane-bound complement inhibitors prevent complement activation. On the pathogenic surfaces, however, the positive amplification loop of the alternative pathway builds up. In this way the “tick over” mechanism of the alternative pathway can distinguish between self and non-self-structures without using pattern recognition molecules.

The cleavage of C3 is the turning point of the complement cascade. This is the point where the three activation routes (classical, lectin and alternative) converge, and the common terminal pathway is initiated. When the density of the deposited C3b increases on the surface the substrate specificity of the C3 convertases switches from C3 to C5 (Mannes 2021). The C5 convertases (C4b2a(C3b)ₙ and C3bBb(C3b)ₙ) cleave C5 into C5b and C5a. The smaller fragment, C5a, is a very potent anaphylatoxin. It stimulates different cells (endothelial, lymphocytes, monocytes, etc.) through G-protein coupled receptors and triggers inflammatory reactions. The larger fragment, C5b, binds C6 and C7. The C5b-7 complex associates with the cell membrane and binds C8. After a conformational change, the C5b-8 complex inserts itself through the membrane of the pathogen (e.g., bacterial membrane) and recruits multiple (as many as 20) C9 molecules. The C5b-9ₙ complex (as generally used in the scientific literature: TCC=terminal complement complex, also known as MAC=membrane attack complex) forms a pore (about 240 Å in diameter) on the cell membrane resulting in the destruction of the cell through osmotic shock and lysis (Tegla 2011). This defense mechanism is very important against Gram-negative bacteria (particularly at Neisseria species) (Petersen 1979, Lewis 2014).

The C1r and C1s of the classical pathway and the MASP-1/-2/-3 of the lectin pathway form a family of proteases with identical domain organization and related functions (Gal 2009). The members of the C1r/C1s/MASPs family consist of six domains: five non-catalytic domains at the N-terminal half of the molecule and a serine protease domain at the C-terminus. The non-catalytic domains are responsible for the protein-protein interactions (e.g., dimerization, tetramer formation, binding to the pattern recognition molecules) while the trypsin-like serine protease domain carries the enzymatic (proteolytic) activity. At the N-terminal region there is a CUB domain (CUB=C1r/C1s, sea urchin Uegf and Bone morphogenetic protein-1), an EGF (EGF=Epidermal Growth Factor) domain, another CUB domain and two CCP (CCP=Complement Control Protein) domains. The CUB1-EGF-CUB2 fragment mediates the dimerization of the MASPs and the binding to the collagenous arms of the pattern recognition molecules. The serine protease domain binds and cleaves the substrates, however, the CCP domains contribute to the substrate specificity via providing additional binding sites (exosites) for the substrates (Kidmose 2012). The CCP1-CCP2-SP fragment is enzymatically identical to the full-length molecule. These proteases (except MASP-3) are present as zymogens in the circulation and become activated only after the pattern recognition part of the complex binds to the activation surface. In vitro both MASP-1 and MASP-2 can autoactivate, but in physiological circumstances the autoactivation capacity of MASP-2 does not manifest. In the blood, MASP-1 is the exclusive activator of MASP-2 (Héja 2012a). The serum concentration of MASP-1 is 143 nM (11 μg/ml), while that of MASP-2 is 6 nM (0.4 μg/ml). The low concentration and the low autoactivation ability of MASP-2 do not allow autoactivation in the blood. On the activation surface the MBL-MASP-1 and MBL-MASP-2 complexes are juxtaposed and because of the high concentration of MASP-1 each MASP-2 molecule is surrounded by multiple MASP-1 molecules (Degn 2014). This arrangement and the enzymatic properties of MASP-1 ensure that MASP-1 acts as an exclusive activator of MASP-2. MASP-1 has an extremely high autoactivation capacity and it cleaves zymogen MASP-2 very efficiently (Megyeri 2013). On the other hand, MASP-2 is very efficient at cleaving of C4 (it is more efficient than C1s). MASP-1 alone cannot initiate the lectin pathway since it cannot cleave C4.

Both MASP-1 and MASP-2 are necessary for lectin pathway activation, and consequently, inhibition of either MASP-1 or MASP-2 results in the inhibition of lectin pathway activation. The low concentration of MASP-2 makes it a more attractive drug target than MASP-1 in the situations where lectin pathway inhibition is beneficial. While MASP-2 has very narrow substrate specificity (it cleaves C4 and C2), MASP-1 has many substrates, but all of them are connected to the innate immune response (Dobó 2016a). MASP-1 has thrombin-like activity (it can cleave fibrinogen, prothrombin, Factor XIII, protease-activated receptors). In this way, MASP-1 could contribute to thrombus formation and directly activate endothelial cells and leukocytes. Moreover, MASP-1 is capable of releasing bradykinin from high molecular weight kininogen and influence the permeability of blood vessels (Dobó 2011, Debreczeni 2019). Recently, it was shown that MASP-2 also can promote thrombus formation, and high plasma levels of MASP-2 can increase the risk of future incident venous thromboembolism (Damoah 2022). Both the MASP1 and MASP2 genes have alternative splicing products. The MASP1 gene encodes three different proteins: MASP-1, MASP-3 and MAp44. The non-catalytic regions (the first five domains) of MASP-1 and MASP-3 are identical, but the serine protease domains are different. Although these proteases bind to the same pattern recognition molecules (MBL, ficolins), due to the different SP domains the enzymatic properties and the biological roles of MASP-1 and MASP-3 are different. MASP-3 cannot autoactivate and its only known physiological substrate is pro-FD. Since the majority (approx. 80%) of MASP-3 is present in activated form in the blood, another protease must be responsible for its activation (Oroszlán 2017). Recently, it was discovered that human proprotein convertases, PCSK6, PC5A and furin, are capable of activating zymogen MASP-3 (Oroszlán 2021). Since secreted PCSK6 (also known as PACE4) is present in the blood, it is very probable that it is the major or the exclusive activator of MASP-3. MASP-3 has no role in lectin pathway activation. The permanent inhibition of MASP-3 results in the inhibition of the alternative pathway (Cummings 2017). Since MASP-3 competes for the recognition molecules with MASP-1 and MASP-2 it could exert some inhibitory effect on the lectin pathway. Similar inhibitory function is attributed to the other alternative splice product of the MASP1 gene: MAp44 (also known as MAP-1) (Pavlov 2012). MAp44 consists of the first four non-catalytic domains (CUB1-EGF-CUB2-CCP1) of MASP-1, and it is expressed mainly in the myocardium. The MASP2 gene also has an alternative splice product: MAp19 (also known as MAP-2, sMAP). MAp19 contains the first two N-terminal domains (CUB1-EGF) of MASP-2 and its biological function is unknown (Stover 1999). Theoretically, it could function as a lectin pathway inhibitor (similarly to MAp44), however, its low serum concentration and its weak binding to the pattern recognition molecules make this assumption questionable.

An intact complement system is indispensable for maintaining the immune homeostasis of the body. It protects against inflammation, and it protects against autoimmune diseases through elimination of immune complexes and cell debris. It is a powerful killing machinery which efficiently targets invading microorganisms while at the same time it spares the healthy human cells. Numerous fluid-phase and cell-surface inhibitors ensure the safe operation of the complement cascade in the human blood. However, when the tight regulatory control of the complement activation is compromised for any reason, inappropriate or uncontrolled activation of the complement system can cause local and/or systemic inflammation, self-tissue damage and development of serious disease conditions. The complement system is now recognized as an attractive therapeutic target to treat various diseases (Dobó 2018, Mastellos 2019, Ricklin 2019).

Ischemia-reperfusion injury (IRI) is a severe autoimmune reaction in which the complement activation plays a major role. When the blood flow in an organ is temporally restricted or interrupted for any reason (e.g., vascular obstruction), the deprivation of oxygen (hypoxia) predisposes the tissues for complement mediated attack after the restoration of blood flow (reperfusion). During reperfusion the immune system recognizes the ischemic cells as damaged self-cells (DAMP=damage associated molecular pattern) and a complex inflammatory reaction is launched in which the complement system plays a cardinal role. IRI significantly contributes to the tissues damage in the case of myocardial infarction and stroke, and it may also cause complications during coronary bypass surgery and organ transplantations (Markiewsky 2007). The lectin pathway is predominantly involved in this process since the pattern recognition molecules (MBL, collectin 11) can recognize certain carbohydrate signatures on ischemic cells (Collard 2000, Nauser 2018). Natural IgM antibodies also bind to certain neoantigens exposed on ischemic tissues, and these IgMs trigger the lectin pathway (Chan 2004, Zhang 2006, McMullen 2006). The alternative pathway amplifies the complement deposition on the infracted tissue. Several experiments in animal models proved that abolition of the lectin pathway activation reduces the extent and the consequences of IRI (Jordan 2001, Hart 2005, La Bonte 2009). Inhibition of MASP-2 is a promising approach to treat or prevent IRI. In mouse models, it was demonstrated that targeting (abolishing or blocking) MASP-2 confers protection from myocardial and gastrointestinal IRI (Schwaeble 2011, Clark 2018). While this protection is MASP-2 dependent, in this mouse model it was found that C4 did not play a role in the MASP-2-mediated IRI. Similarly, in the case of renal IRI the tissue damage was MASP-2 dependent, but C4 independent in the mouse model (Asgari 2014). The natural, endogen lectin pathway inhibitor (MAp44) was also effective in attenuating myocardial IRI (Pavlov 2012). MAp44, as a non-catalytic fragment of MASP-1/3 is capable of displacing MASP-1 and MASP-2 from the pattern recognition molecules. In a mouse model, where the animal expresses human MBL, anti-MBL antibodies reduced the size of myocardial IRI (Pavlov 2015). The protecting effect of lectin pathway inhibition was also demonstrated in a renal IRI model in pigs, as well (Castellano 2010). Furthermore, targeting MASP-2 in mice mediated protection against post-ischemic brain injury, as well (Orsini 2016). In line with this preclinical finding, smaller infarction size and better functional outcomes were revealed also in MBL deficient patients after ischemic stroke (Osthoff 2011). In summary, the above-described results strongly suggest that inhibition of the lectin pathway and particularly inhibition of the MASP-2 enzyme can prevent or alleviate IRI of different organ systems in various animal models as well as in human patients.

Trauma-related and sepsis-related tissue damages also lead to massive complement activation. The complement overactivation and the “protease storm” during sepsis fuel a vicious hyperinflammatory cycle leading to multiple organ failure. As complement inhibition may be beneficial (Liu 2007), C1-inhibitor is being tested in trauma (Igonin 2012, van Erp 2021). C1-inhibitor is the principal inhibitor of the classical and the lectin pathways and it also inhibits plasma kallikrein. Plasma kallikrein and MASP-1 are responsible for releasing the vasoactive peptide bradykinin from high molecular weight kininogen (Dobó 2011).

Artificial materials used in modern medicine such as polymer plastics, metal alloys, nanoparticles (contrast agents and drug carriers, especially liposomes, lipid nanoparticle vaccines) can activate the complement system and cause an allergy-like reaction called CARPA (complement activation-related pseudoallergy) (Szebeni 2005, Dézsi 2022). CARPA is independent of IgE, but its mechanism is not fully understood yet. As the pattern recognition molecules may recognize the artificial surfaces, inhibition of the lectin pathway could be a therapeutical option.

Haemodialysis is an indispensable treatment of patients suffering in severe renal diseases or even in end-stage renal disease (ESRD). During haemodialysis the blood of the patient contacts heavily with the artificial surface of the haemodialysis filter unit. This contact can trigger complement activation, exacerbate inflammation, and contribute to cardiovascular diseases (Ekdahl 2017). Although the introduction of synthetic polymers in the haemodialysis filters reduced the extent of inflammation compared to the cellulose-based filters, unwanted complement activation is still a problem (Ekdahl 2011). Applying of complement inhibitors during haemodialysis can alleviate the inflammatory symptoms and improve the quality of life of the patients.

The pathological complement activation is a decisive factor in many renal disorders. The kidney is especially vulnerable for complement mediated attacks due to its unique anatomical and functional features. In the case of C3 glomerulophathies (C3G) uncontrolled complement activation results in C3 deposition in the glomeruli without immunoglobulin deposition (Fakhouri 2010, Pickering 2013). There are two major subgroups of C3G: dense deposit disease (DDD) and C3 glomerulonephritis (C3GN). In the case of membranoproliferative glomerulonephritis, classical pathway activation contributes to C3 deposition, since immunoglobulins and C1q are also deposited in the kidney. In the case of IgA nephropathy polymeric IgA1 triggers the activation of the lectin and alternative pathways.

Primary membranous nephropathy (pMN) is the most common cause of nephrotic syndrome in non-diabetic Caucasian adults over 40 years of age. It has an estimated incidence of 8-10 cases per 1 million. Recently, it has been demonstrated that the activation of the lectin pathway plays a key role in the etiology of pMN. In pMN anti-PLA2R1 antibodies elicit proteolysis of the two essential podocyte proteins synaptopodin and NEPH1, resulting in perturbations of the podocyte cytoskeleton. Anti-PLA2R1-IgG4 directly binds to MBL in a glycosylation-dependent manner. In a cohort of pMN patients, increased levels of galactose-deficient IgG4 were identified, which correlated with anti-PLA2R1-titers and podocyte damage induced by patient sera. The aberrantly glycosylated IgG4-induced lectin pathway activation could be abolished by MASP-1- and MASP-2-specific inhibitors. It was suggested that blockade of the lectin pathway could be a new and effective therapeutical option to treat pMN (Haddad 2021).

Atypical hemolytic uremic syndrome (aHUS) is a complement-related disease manifesting in microangiopathic hemolytic anemia, thrombocytopenia, vascular damage with thrombosis, and organ injury, typically that of the kidney (Noris 2009). The complement system attacks the kidney endothelium promoting the formation of microthrombi in the renal microvasculature. The development of aHUS is associated with uncontrolled complement activation due to mutations in (Neumann 2003) or autoantibodies against (Hofer 2014) Factor H, the master regulator of the alternative pathway.

Hemolytic uremic syndrome (HUS) can also be elicited by bacterial infections. Bacterial toxins (e.g., Shiga-toxin) compromise the regulation of the complement cascade resulting in uncontrolled activation of the complement system (Conway 2015). Inhibition of the lectin pathway of complement activation provided protection against HUS in a mouse model of HUS (Ozaki 2016).

Besides the kidney, another organ which is extremely vulnerable for pathological complement activation is the eye. Age-related macular degeneration (AMD) is a chronic inflammatory disease of the retina that represents a leading cause of irreversible vision loss in the industrialized world (Geerlings 2017). It is estimated that it affects a large population (about 100 million AMD cases) worldwide. In the center of the retina of AMD patients, immune deposits called drusens accumulate underneath the retinal pigment epithelium. The drusens (that contain activated complement components) compromise the transport of oxygen and nutrients to the photoreceptors facilitating their degeneration. Applying complement inhibitors in the treatment of AMD is under clinical testing (Fritsche 2016).

The involvement of lectin pathway activation has also been implicated in the pathogenesis of rheumatoid arthritis (Ammiztboll 2012) and also in juvenile idiopathic arthritis (Petri 2015). Rheumatoid arthritis is an extremely complex disorder whose pathomechanism is not revealed yet. The proteolytic activity of MASP-1 and MASP-2 may contribute to the progression of the disease (Holers 2018).

Excessive activity of the complement system also plays a role in the development and maintenance of various neurodegenerative diseases (e.g., Alzheimer's, Huntington's and Parkinson's diseases and Multiple Sclerosis) (Tichaczek-Goska 2012; Ingram 2009). The complement system is responsible for synapse elimination during normal postnatal brain development. If this process is pathologically upregulated during adulthood it can lead to development of neurodegenerative diseases (Presumey 2017). The pathological activation of the lectin pathway in the central nervous system can contribute to the development of schizophrenia, as well (Mayilyan 2006).

Paroxysmal nocturnal haemoglobinuria (PNH) is a rare complement-related disorder (Hill 2017). Acquired somatic mutations in genes responsible for membrane anchor synthesis result in the lack of two membrane-bound regulator proteins: DAF (CD55) and CD59. In PNH patients, erythrocytes have increased susceptibility to (bystander) complement attack, which leads to their lysis (intravascular haemolysis) and contributes to thrombotic complications. Complement inhibition is a successful strategy to treat PNH (Gavriilaki 2022).

Inhibition of the lectin pathway of complement activation may also mediate protection against graft rejection after organ transplantation (Fildes 2008; Ibernon 2014).

Uncontrolled, excessive complement activation can cause severe complications during viral infections. While the complement system contributes to the elimination of viruses and virus-infected cells, overactivation of the complement system can trigger harmful inflammatory reactions. An example is the COVID-19 pandemic caused by the severe acute respiratory syndrome coronavirus 2 (SARS-CoV-2). A primary cause of death in COVID-19 is severe respiratory failure. Pathological activation of the lectin pathway can result in arterial thrombosis and severe endothelial damage in the lung tissue (Magro 2020). The lectin pathway plays a cardinal role in this process (Götz 2022). Coronavirus spike (S) protein and nucleocapsid (N) protein have been shown to activate the lectin pathway. It has been reported that N-protein directly activates MASP-2 (Ali 2021). In the lung and kidney tissues of the deceased patients, heavy MASP-2 deposition has been detected (Niederreiter 2022). Inhibition of MASP-2 could be beneficial in coronavirus infection through reducing inflammation, endothelial damage, and thrombosis (Rambaldi 2020, Flude 2021).

Since the complement system is potentially harmful it is tightly regulated in the blood. Both fluid phase and membrane-anchored inhibitors ensure that the complement system does not damage self-tissues. The major inhibitor of the early proteases of the classical and the lectin pathway is C1-inhibitor (Davis 2010). C1-inhibitor is a serpin (serine protease inhibitor) which acts as a pseudo-substrate and makes an irreversible covalent complex with the proteases. C1-inhibitor inhibits C1r, C1s, MASP-1 and MASP-2. MASP-3 is not inhibited by C1-inhibitor and there is no known physiological inhibitor of this protease. Although C1-inhibitor has anti-inflammatory properties, it is not specific; it inhibits several pathways at the same time. Besides the complement system C1-inhibitor inhibits the coagulation, the contact and the fibrinolytic systems. In some cases, it could be beneficial; however, it could cause complete immune suppression. In many cases pathway specific inhibition is preferred, since in this case the disease-causing pathway is blocked while other pathways can maintain their protective functions against infections. Precluding lectin pathway activation could prevent development of certain diseases without interfering with the protective function of the classical and the alternative pathways. Another serpin, antithrombin, is also an efficient inhibitor of the lectin pathway in the presence of heparin (Paréj 2013). Heparin itself is able to lessen lectin pathway activation to a certain extent. It was also suggested that alfa2-macroglobulin can inhibit the lectin pathway, but this issue is controversial. The blood borne canonical inhibitor TFPI (tissue factor pathway inhibitor) is a very weak inhibitor of MASP-2 (Keizer 2015). Considering its low serum concentration (2.5 nM) it has a negligible effect on the lectin pathway activation at physiological setting.

As outlined above, inhibition of the unwanted, inappropriate complement activation could have therapeutical effect in many clinical conditions. The trypsin-like complement proteases can effectively be inhibited by small organic molecules (e.g., benzamidine, NPGB, FUT-175). These compounds, however, are not specific enough; they inhibit all the trypsin-like proteases (thrombin, plasmin, kallikrein) in the other cascade systems in the blood. In many cases pathway-specific inhibition is required to treat a certain disease without causing severe side effects. Peptide and protein inhibitors make numerous contacts with the protease ensuring a specific and efficient inhibition. Inhibition of the lectin pathway can be carried out through the inhibition of either MASP-1 or MASP-2. Due to its low serum concentration MASP-2 is an ideal target for lectin pathway inhibition.

International patent application WO2010136831 discloses oligopeptides that are inhibitors of the MASP enzymes, selectively inhibiting the lectin pathway. Some of them were selective MASP-2 inhibitors over the MASP-1 enzyme while some of them were not selective between MASP-1 and MASP-2. The oligopeptides described in that prior art are of plant origin, i.e., those peptides were evolved by the phage display technique from the 14-amino acid length Sun Flower Trypsin Inhibitor (SFTI) and termed as SFTI-based MASP Inhibitors (SFMI) (Kocsis 2010).

International patent application WO2012007777 discloses proteins that have certain MASP inhibitory sequence. These sequences were evolved also by the phage display technique starting from the sequence of the inhibitory loop of the S. gregaria Chymotrypsin Inhibitor (SGCI). The MASP inhibitors described in that prior art and termed as SGCI-based MASP Inhibitors (SGMI) are of insect origin and are selective either for MASP-1 or for MASP-2 (Héja 2012b).

International patent application WO2011047346 discloses MASP-2 inhibitors for the treatment of complement mediated coagulation disorder.

Inhibitory oligopeptides are often inserted in proteins, i.e., host proteins, to keep the functional structure of the peptide intact and to prevent decomposition by proteases or by other factors. A generally used choice of this kind of host proteins is the protease inhibitor called Kunitz domain type protein, or shortly Kunitz domain. Kunitz domain type proteins are widely used for this purpose as they are stable and easy to produce. Such modified Kunitz domains are useful biopharmaceuticals acting as specific protease inhibitors. U.S. Pat. No. 5,994,125A discloses Kunitz domain type proteins that inhibit the serine protease human plasma kallikrein. Definition, features and use of Kunitz domains are described in U.S. Pat. No. 5,994,125A, which is therefore hereby incorporated by reference in its entirety.

International patent application WO2018127719 discloses human protein based compounds that are efficient and selective inhibitors of the human MASP-2 enzyme. Unlike international patent applications WO2010136831 and WO2012007777 that both disclosed non-human peptide or protein based MASP-2 inhibitor compounds, the human protein based compounds of WO2018127719 should impose a significantly lower risk of immunogenicity in humans.

The proteins according to the invention disclosed in WO2018127719 are based on the Kunitz-type scaffold of the second domain (residues 121-178) of the human Tissue Factor Pathway Inhibitor-1 protein (TFPI-1; UniProt ID P10646), hereafter referred to as TFPI-D2 (SEQ ID NO: 116).

The general Kunitz domain sequence (SEQ ID NO: 1) defined in the patent application U.S. Pat. No. 5,994,125A is the following:

xxxxCxxxxxxGxCxxxxxxXXXxxxxxxCxxFxXXGCxXxxxxxxxxxx
CxxxCxxx

As disclosed in WO2018127719, the bold (xCxxxxx) segment within the general Kunitz domain sequence indicates the position which was substituted by certain amino acid sequences resulting in a protein that inhibits human MASP-2 enzyme. The one-residue longer Kunitz domain segment GxCxxxxx in the Kunitz domain forms a disulphide stabilized surface loop. This, so called canonical inhibitory loop, occupies the substrate binding groove of MASP-2 as described below.

The Kunitz domain family belongs to the large group of substrate-like reversible protease inhibitors that has at least 18 independently evolved families. While each family has a distinct scaffold, all of them have a surface inhibitory loop that blocks the substrate-binding groove of the enzyme in essentially the same, i.e., canonical conformation.

Residue positions of the substrate-like canonical inhibitory loop are referred to by the nomenclature of Schechter & Berger 1967 originally introduced for peptide substrates. The scissile peptide bond is formed between the carbonyl group of the P1 and the amino group of the P1′ residues. The canonical inhibitory loop is generally located within the 8-residue long P4-P4′ segment. In the case of the general Kunitz domain sequence (SEQ ID NO: 1), the P4-P4′ segment corresponds to GxCxxxxx (see Table 100), where the highly conserved x12 glycine (Kunitz numbering in SEQ ID NO: 1) occupies the P4 position, and the highly conserved x14 cysteine (Kunitz numbering in SEQ ID NO: 1) occupies the P2 position. Both said conserved Kunitz domain residues have key importance in forming and stabilizing the canonical conformation of the inhibitory loop.

TABLE 100
Numbering concept of the P4-P4′ segment in the Kunitz domain

| | position names in the P4-P4′ segment | | | | | | | |
|---|---|---|---|---|---|---|---|---|
| | P4 | P3 | P2 | P1 | P1′ | P2′ | P3′ | P4′ |
| P4-P4′ segment | G | x | C | x | x | x | x | x |
| sequence * | | | | | | | | |
| positions in SEQ | 12 | 13 | 14 | 15 | 16 | 17 | 18 | 19 |
| ID NO: 1 | | | | | | | | |

* x denotes a variable position

Among the inhibitors disclosed in WO2018127719 the highest efficiency human MASP-2 inhibiting compound (SEQ ID NO: 11 in WO2018127719) is the one with the canonical inhibitory loop sequence of GFCRAVKR which inhibits human MASP-2 with a 2 nM inhibitory binding constant (K_{I}) value, but hardly inhibits rat MASP-2 (the K_{I} value is 640 nM), making it unusable in rat animal models. Another inhibitor with the inhibitory loop sequence of GPCRAVKR disclosed in WO2018127719 with SEQ ID NO: 14 is only about 4-times weaker inhibitor of human MASP-2 and has a K_{I} of 7.9 nM, however, it is an equally good inhibitor of rat MASP-2 (K_{I}=7.2 nM).

While nanomolar inhibitory binding constants of the abovementioned two inhibitors are generally considered to represent high affinity, they might still not be high enough for all types of drug development purposes.

Therefore the goal of the present invention was to develop significantly higher binding affinity compounds and therefore significantly higher potency lead molecules. To reach this goal a structure-based directed evolution campaign was conducted that started from the above described inhibitor (in WO2018127719 referred to as SEQ ID NO: 14), which will be referred to as EVO2 (SEQ ID NO: 2) in the present description. These efforts indeed led to the development of certain amino acid residue sets at certain positions outside of the canonical inhibitory loop, which dramatically increased the affinity and inhibitory potency of the previously developed above described MASP-2 inhibitors. Namely, the essence of the present invention is not in the canonical inhibitory loop, but in the host protein sequence outside the canonical inhibitory loop.

### SUMMARY OF THE INVENTION

The present invention relates to modified Kunitz domain proteins having a general amino acid sequence set forth in the general sequence of SEQ ID NO: 115, with certain provisions.

The present invention is based on the surprising finding that two Kunitz domain residues at exactly defined Kunitz domain positions can synergistically enhance MASP-2 inhibitory potency compared to the compounds disclosed in WO2018127719. Further positions in the Kunitz domain were also identified, where certain amino acids further enhance efficacy.

One of the two above mentioned positions is the non-conserved position 17 according to the numbering of SEQ ID NO: 1 (i.e., the general Kunitz sequence numbering), which occupies the P2′ position of the P4-P4′ segment (see Table 100 above). This position corresponds to the X₃ position in the general formula Ih GX₁CRX₂X₃X₄X₅ according to the invention disclosed in WO2018127719. At this X₃ position, the general formula Ih of WO2018127719 allowed for V, A, I, L, M, D, H and S amino acid residues.

In the different sequential context of the present invention, a different, but overlapping set of amino acid residues were found at this position 17 to be preferred for human MASP-2 binding. Based on their higher than the expected mean value of 5% codon normalized frequency in the set of two-hundred-ninety human MASP-2 binding clones, L (14%), I (14%), F (13%), Y (11%), A (10%), M (7%), V (6%) and H (6%) were found to be the overrepresented amino acid types as illustrated in Table 9. Out of these eight amino acid types, L, I, F, Y and A are the most preferred ones.

On one hand, the present invention limits the original set described in WO2018127719 to A, I and L, (i.e., excluding V, M, D, H and S). On the other hand, the present invention broadens this set with two amino acids, namely with F and Y. The resulted set with five amino acids is markedly hydrophobic. For the present invention we define this modified amino acid set as the “17-set”, meaning that in position 17 according to the numbering of SEQ ID NO: 1 the following amino acids can be present: A, I, L, F and Y.

Accordingly, because of the modified X₃ amino acid set (compared to WO2018127719), i.e., the new (17-set), for the purpose of the present invention we define a modified general formula Ih-mod as follows:

(Ih-mod)
GX₁CX_{1V}X₂X₃X₄X₅,

where
X₁ is F, Y, L, P, Q, M, V, W, A, T and
X_{1V} is R, K and
X₂ is A, G, S, T and
X₃ is A, I, L, F, Y and
X₄ is K, I, Q, R, H, S, F, M, N, L, V and
X₅ is R, V, I, K, M, Q, E, F, L, N, Y, D, S, H,
where the possible amino acids in the X₃ position form the 17-set. As later will be detailed, the amino acid sequence of the general formula Ih-mod is present within the general Kunitz domain sequence, starting at position 12 and ending at position 19 of SEQ ID NO: 1.

The other position is the non-conserved position 34 according to the numbering of SEQ ID NO: 1 (i.e., the general Kunitz sequence numbering). This position is not in sequential vicinity of the P4-P4′ segment and therefore is referred to as an exosite position.

As illustrated in Table 3, we found that when position 17 can contain only L or V, human MASP-2 prefers the following six amino acid types at position 34: Y (17%), I (14%), S (11%), F (10%), L (8%) and H (7%), while, as illustrated in Table 9, when position 17 is allowed to contain any of the 20 amino acids, human MASP-2 prefers the following five amino acid types at position 34, Y (18%), I (17%), F (14%), G (6%) and V (6%). The combined set contains the following eight preferred amino acid types at position 34: Y, I, F, G, V, S, L and H. From this set six residues, namely Y, I, F, G, V and S are the most preferred ones at position 34. This amino acid set is dominantly hydrophobic like the amino acids of the above mentioned 17-set, except S34, which, just like Y34, has a hydroxyl group suggesting a complex stabilizing potency of that hydroxyl. For the present invention we define this modified amino acid set as the “34-set”, meaning that at position 34 according to the numbering of SEQ ID NO: 1 the following amino acids can be present: Y, I, F, G, V and S.

Surprisingly, we found that when any of the tested 17-set and 34-set combinations out of the thirty pairs was introduced into Kunitz domain based proteins disclosed in WO2018127719, they increased MASP-2 binding affinity and lectin pathway inhibitory potency of those compounds up to or over 40-fold.

Therefore, the present invention relates to a protein comprising an amino acid sequence of SEQ ID NO: 115, where the variable positions in the amino acid sequence of SEQ ID NO: 115 are limited in such a way that
x1 to x4, x58, x57, and x56 may be variable or absent,
x6 to x11, x13, x15 to x20, x24 to x29, x31 to x32, x34, x39, x41 to x42, x44, x46 to x50, x52 to x54 may be variable,
x21 is F, Y, or W,
x22 is Y or F,
x23 is Y or F,
x35 is Y or W,
x36 is G or S,
x40 is G or A,
x43 is N or G, and
x45 is F or Y;
characterized in that said protein
i) has an amino acid sequence segment of general formula Ih-mod, starting at position 12 and ending at position 19 of SEQ ID NO: 115:

(Ih-mod)
GX₁CX_{1V}X₂X₃X₄X₅,

where
X₁ is any of F, Y, L, P, Q, M, V, W, A, or T,
X_{1V} is R, or K,
X₂ is any of A, G, S, or T,
X₃ is any amino acid of the 17-set, where the 17-set comprises A, I, L, F, or Y,
X₄ is any of K, I, Q, R, H, S, F, M, N, L, or V, and
X₅ is any of R, V, I, K, M, Q, E, F, L, N, Y, D, S, H; and
ii) in position 34 of the amino acid sequence of SEQ ID NO: 115 it contains any amino acid of the 34-set, where the 34-set comprises Y, I, F, G, V and S;
and to salts, esters and pharmaceutically acceptable prodrugs of said protein.

According to a preferred embodiment, said protein, salts, esters and pharmaceutically acceptable prodrugs of said protein is a human MASP-2 inhibitor with a K_{I} value equal to or lower than 100 nM.

According to a preferred embodiment, said protein comprises an amino acid sequence, where
i) said amino acid sequence has at least 70%, or at least 80%, or at least 90%, or at least 95% similarity, more preferably at least 98% similarity, even more preferably at least 70%, or at least 80%, or at least 90%, or at least 95% identity, most preferably 98% identity with the amino acid sequence set forth in SEQ ID NO: 116, with the proviso that
i) said amino acid segment starting at position 12 and ending at position 19 has the sequence defined by the general formula Ih-mod; and
ii) in position 34 of the amino acid sequence of SEQ ID NO: 115 it contains an amino acid selected from the 34-set.

According to a preferred embodiment, said protein comprises an amino acid sequence, where the amino acid pair from the 17-set and 34-set is selected from the group consisting of (in x17/x34 format): A/Y, A/I, A/F, A/G, A/V, A/S, I/Y, I/I, I/F, I/G, I/V, I/S, L/Y, L/I, L/F, L/G, L/V, L/S, F/Y, F/I, F/F, F/G, F/V, F/S, Y/Y, Y/I, Y/F, Y/G, Y/V, Y/S.

According to a more preferred embodiment, said amino acid pair from the 17-set and 34-set is selected from the group consisting of (in x17/x34 format): A/Y, A/I, A/F, A/V, I/Y, I/I, I/F, I/G, I/V, I/S, L/Y, L/I, L/F, L/G, L/V, L/S, F/I, F/G, F/V, F/S, Y/Y, Y/I, Y/G, Y/V, Y/S.

According to a further preferred embodiment, said protein comprises an amino acid sequence, where in position 9 of the amino acid sequence of SEQ ID NO: 115 said protein contains any amino acid of the 9-set, where the 9-set consists of N or E.

According to a further preferred embodiment, said protein comprises an amino acid sequence, where in position 39 of the amino acid sequence of SEQ ID NO: 115 said protein contains any amino acid of the 39-set, where the 39-set consists of F or L.

According to a further preferred embodiment, said protein comprises an amino acid sequence, where in position 46 of the amino acid sequence of SEQ ID NO: 115 said protein contains any amino acid of the 46-set, where the 46-set consists of V or E.

According to another preferred embodiment, said protein is selected from proteins comprising an amino acid sequence selected from the group consisting of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 54, SEQ ID NO: 55, and SEQ ID NO: 56.

According to a further preferred embodiment, said protein comprises an amino acid sequence that has at least 70%, or at least 80%, or at least 90%, or at least 95% similarity, more preferably at least 98% similarity, even more preferably at least 70%, or at least 80%, or at least 90%, or at least 95% identity, most preferably 98% identity, or is fully identical with any of the amino acid sequences set forth from SEQ ID NO: 3 to SEQ ID NO: 22 and from SEQ ID NO: 24 to SEQ ID NO: 32, with the proviso that the amino acid segment starting at position 12 and ending at position 19 has the sequence defined by the general formula Ih-mod, and in position 34 of the amino acid sequence of SEQ ID NO: 115 it contains an amino acid selected from the 34-set.

According to a further preferred, said protein is selected from proteins comprising any of the amino acid sequences of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 24, SEQ ID NO: 25, and SEQ ID NO: 26, SEQ ID NO: 27, SEQ ID NO: 28, SEQ ID NO: 29, SEQ ID NO: 30, SEQ ID NO: 31, and SEQ ID NO: 32.

According to an even more preferred embodiment, said protein comprises an amino acid sequence that has at least 95% similarity, more preferably at least 98% similarity, even more preferably 95% identity, most preferably 98% identity, or is fully identical with any of the amino acid sequences set forth in SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 24, SEQ ID NO: 25, and SEQ ID NO: 26, with the proviso that the amino acid segment starting at position 12 and ending at position 19 has the sequence defined by the general formula Ih-mod, and in position 34 of the amino acid sequence of SEQ ID NO: 115 it contains an amino acid selected from the 34-set.

According to another embodiment of the present invention, said protein is in the form of a fusion protein, which comprises
i) an amino acid sequence of SEQ ID NO: 115, where the variable positions in the amino acid sequence of SEQ ID NO: 115 are limited in such a way that
x1 to x4, x58, x57, and x56 may be variable or absent,
x6 to x11, x13, x15 to x20, x24 to x29, x31 to x32, x34, x39, x41 to x42, x44, x46 to x50, x52 to x54 may be variable,
x21 is F, Y, or W,
x22 is Y or F,
x23 is Y or F,
x35 is Y or W,
x36 is G or S,
x40 is G or A,
x43 is N or G, and
x45 is F or Y;
within which
   a) there is an amino acid sequence segment of general formula Ih-mod, starting at position 12 and ending at position 19 of said SEQ ID NO: 115:

(Ih-mod)
GX₁CX_{1V}X₂X₃X₄X₅,

where
X₁ is any of F, Y, L, P, Q, M, V, W, A, or T,
X_{1V} is R, or K,
X₂ is any of A, G, S, or T,
X₃ is any amino acid of the 17-set, where the 17-set comprises A, I, L, F, or Y,
X₄ is any of K, I, Q, R, H, S, F, M, N, L, or V, and
X₅ is any of R, V, I, K, M, Q, E, F, L, N, Y, D, S, H; and
b) in position 34 of the amino acid sequence of SEQ ID NO: 115 it contains an amino acid selected from the 34-set, where the 34-set comprises Y, I, F, G, V and S; and
ii) an antibody Fc-domain, preferably a human antibody Fc-domain.

According to a preferred embodiment, said fusion protein comprises an amino acid sequence that has at least 70%, or at least 80%, or at least 90%, or at least 95% similarity, more preferably at least 98% similarity, even more preferably at least 70%, or at least 80%, or at least 90%, or at least 95% identity, most preferably 98% identity, or is fully identical with SEQ ID NO: 114.

The present invention also relates to pharmaceutical preparations that contain at least one protein, its pharmaceutically acceptable salt, pharmaceutically acceptable ester or pharmaceutically acceptable prodrug of the present invention, and at least one additive. Said at least one protein is preferably selected from proteins defined by any of the amino acid sequences of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 54, SEQ ID NO: 55, and SEQ ID NO: 56; more preferably said at least one protein is selected from the proteins comprising any of the amino acid sequences of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 24, SEQ ID NO: 25, and SEQ ID NO: 26, SEQ ID NO: 27, SEQ ID NO: 28, SEQ ID NO: 29, SEQ ID NO: 30, SEQ ID NO: 31, and SEQ ID NO: 32; most preferably said at least one protein is selected from the proteins comprising any of the amino acid sequences of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 24, SEQ ID NO: 25, and SEQ ID NO: 26.

Said additive is preferably a matrix ensuring controlled active agent release.

The pharmaceutical preparations according to the present invention are preferably in the form of infusions, tablets, powders, granules, suppositories, injections, syrups, inhalation and intranasal delivery systems.

Nucleic acid encoding any of the proteins of the present invention defined above.

Vector, comprising said nucleic acid.

Kits containing at least one protein, its salt or ester of the present invention, and manual for use or reference to such manual.

Screening procedure of compounds potentially inhibiting a MASP-2 enzyme, preferably the human MASP-2 enzyme, in the course of which i) a protein according to the present invention, its salt or ester, in a labelled form, is added to a solution containing said MASP-2 enzyme, preferably said human MASP-2 enzyme, then ii) the solution containing one or more compounds to be tested is added to it, and iii) the amount of the released labelled protein is measured.

Use of proteins, their salts, esters or prodrugs, of the present invention for the inhibition of MASP-2 protein, preferably human MASP-2 protein.

Use of proteins, their pharmaceutically acceptable salts, pharmaceutically acceptable esters or pharmaceutically acceptable prodrugs, of the present invention in the production of a pharmaceutical preparation suitable for the treatment or prevention of diseases that can be treated by inhibiting the complement system.

Said diseases are preferably selected from the following list: (1) ischemia-reperfusion (IR) injuries (especially following recanalization after arterial occlusion due to thrombosis or other obstructive diseases), including those occurring after myocardial infarction (e.g., treated by percutaneous coronary interventions or thrombolysis), coronary bypass surgery, IR injury of the graft at organ transplantations, gastrointestinal IR injury, renal IR injury, post-ischemic brain injury, stroke, thrombosis affecting any region of the body; (2) inflammatory and autoimmune conditions with excess activation of the complement system, including autoimmune nephritis (including dense deposit disease, C3 glomerulonephritis), IgA nephropathy, membranous nephropathy, rheumatoid arthritis (RA), juvenile idiopathic arthritis, age-related macular degeneration, systemic lupus erythematosus (SLE), atypical hemolytic uremic syndrome (aHUS), thrombotic microangiopathy (TMA), post-infection hemolytic uremic syndrome (HUS), pseudo-allergy developing as a consequence of complement activation (CARPA), paroxysmal nocturnal hemoglobinuria (PNH), polytrauma, graft rejection after organ transplantation; venous thromboembolism (3) neurodegenerative diseases, preferably Alzheimer's disease, Huntington's disease, Parkinson's disease, multiple sclerosis and age-related macular degeneration; (4) complement overactivation caused by viral infection such as COVID-19 (SARS-CoV-2), acute respiratory distress syndrome (ARDS), complement associated microvascular injury and thrombosis due to severe COVID-19 infection.

Process for isolating the human MASP-2 enzyme, in the course of which i) a carrier with one or more immobilised proteins, their pharmaceutically acceptable salts, esters, of the present invention are contacted with a solution containing said human MASP-2 enzyme and ii) the preparation is washed.

### BRIEF DESCRIPTION OF THE DRAWINGS

In the drawings

FIG. 1 shows a schematic representation of the phage display method used for evolving the inhibitors of the present invention;

FIG. 2 shows the DNA (SEQ ID NO: 117) and amino acid (SEQ ID NO: 118) sequence of the fusion gene created for the display of TFPI-D2 on the surface of M13 bacteriophage;

FIG. 3 shows the sequence logo diagrams representing sets of unique sequences obtained in the first stage of evolution by selecting the first library on the human or the rat MASP-2 enzyme;

FIG. 4 shows the sequence logo diagrams representing sets of unique sequences obtained in the second stage of evolution by selecting the second library on the human or the rat MASP-2 enzyme;

FIG. 5 shows the sequence logo diagrams representing sets of unique sequences obtained in the third stage of evolution by selecting the third library on the human or the rat MASP-2 enzyme, and it shows an additional sequence logo corresponding to all human MASP-2 binding clones regardless whether selected on human or rat MASP-2;

FIG. 6 shows the cumulative side chain volume distribution of x17/x34 residue pairs;

FIG. 7 shows the vector map of the pS100A4-EVO24 bacterial expression plasmid;

FIG. 8 shows the vector map of pEW-EVO24L bacterial expression plasmid;

FIG. 9 shows the DNA and protein sequence of the EVO24L chimera protein (SEQ ID NO: 114);

FIG. 10 shows the pharmacodynamic effects of the selected compounds in rat.

### DETAILED DESCRIPTION OF THE INVENTION

The inhibition of the complement system, including the lectin pathway, may be an efficient tool in fighting against human diseases occurring as a result of the abnormal activity of the complement system.

The presently known selective lectin pathway blocker canonical inhibitors have either the plant-originated SFTI peptide structure (see WO2010136831, SFTI-based SFMI and the inhibitors are described) or have the insect-originated Pacifastin protein structure (see WO2012007777, where Pacifastin-based SGMI inhibitors are described), or have a human Kunitz domain scaffold (see WO2018127719). While the last mentioned TFMI inhibitors shall impose a significantly lower risk of immunogenicity in the human host than the previous inhibitors having non-human scaffold, it seemed possible that their efficacy could be significantly improved by a limited number of amino acid replacements by involving scaffold positions distinct from those occupied by the residues of the general (Ih) sequence described in WO2018127719. We pursued and achieved this goal by identifying amino acid replacements that increase the MASP-2 binding strength (lower the KD) of the corresponding compounds and enhance their lectin pathway blocking efficacy.

We surprisingly found that the Kunitz domain protein based compounds having the sequence of modified general formula Ih-mod combined with the 34-set, further optionally combined with the 9-set, the 39-set and/or the 46-set meet the objective of the present invention, i.e., they are more efficient inhibitors of the human MASP-2 enzyme than those described in WO2018127719.

As used in the present description, Kunitz domain proteins have the general Kunitz domain sequence (SEQ ID NO: 1) as defined in U.S. Pat. No. 5,994,125A:

xxxxCxxxxxxGxCxxxxxxXXXxxxxxxCxxFxXXGCxXxxX
xXxxxxxCxxxCxxx

where:
x1 to x4, x58, x57, and x56 may be variable or absent,
x6 to x11, x13, x15 to x20, x24 to x29, x31 to x32, x34, x39, x41 to x42, x44, x46 to x50, x52 to x54 may be variable,
X21=Phe, Tyr, Trp,
X22=Tyr or Phe,
X23=Tyr or Phe,
X35=Tyr or Trp,
X36=Gly or Ser,
X40=Gly or Ala,
X43=Asn or Gly, and
X45=Phe or Tyr.

The proteins of the present invention are defined via the amino acid segment of SEQ ID NO: 115, with certain limitations.

Although the present invention relates to human MASP-2 inhibitors, the way towards the invention included the research on rat MASP-2 inhibitors, as well. The latter research part aimed to reveal, which human MASP-2 inhibitors could also inhibit rat MASP-2. These bispecific inhibitors are useful for in vivo studies performed in rat as animal model. The research work cannot be divided into “human” and “rat” parts. Nevertheless, besides describing the development of human MASP-2 inhibitors, the description below also contains references to the development of rat MASP-2 inhibitors. The sole purpose of the rat MASP-2 related information is to provide a full support to the present invention.

If “sequence” is mentioned in the present description without a prefix of “amino acid” or “nucleic acid”, an amino acid sequence shall be understood.

The general formula Ih-mod, as well as, the 9-set, the 34-set, the 39-set and the 46-set as used herein describes amino acid sequences or amino acid sets using the one-letter code of amino acid residues known by a person skilled in the art. The positions of the eight-unit long P4-P4′ segment (see Table 100 above) sequences of the general formula Ih-mod are denoted by X₁ to X₅ (incl. X_{1V}) in case the amino acid at said position is variable, and are denoted by a certain one-letter code (e.g., G, C or R) if it is constant. The possibilities in positions X₁ to X₅ are shown with the one-letter codes. For example, if in case of general formula Ih-mod X₂ is said to be A, G, S, T, it means that alanine, glycine, serine and threonine may be the choice in position X₂. We used the IUPAC recommendations to mark the amino acid side chains in the given sequences (Nomenclature of α-Amino Acids, Recommendations, 1974—Biochemistry, 14 (2), 1975).

The present invention relates to Kunitz domain proteins. Under Kunitz family or Kunitz domain the following shall be understood within the scope of the present invention. The already referred U.S. Pat. No. 5,994,125A gives a detailed description of the Kunitz domain. Briefly, Kunitz domain means a homologue of bovine pancreatic trypsin inhibitor, hereinafter BPTI (not of the Kunitz soya-bean trypsin inhibitor). A Kunitz domain is a domain of a protein having at least 51 amino acids (and up to about 61 amino acids) containing at least two, and preferably three, disulfides. Herein, the residues of all Kunitz domains are numbered as 1-58 by reference to the 58 amino acid residue mature form of BPTI, the amino-acid sequence of which was disclosed as SEQ ID NO: 21 in U.S. Pat. No. 5,994,125A. Note, that the full-length, prepro form of BPTI contains 100 amino acid residues, and the 58-residue matured segment corresponds to the segment of 36-93 according to the full-length protein numbering. We note here that the sequence of mature BPTI disclosed in Table 2 of U.S. Pat. No. 5,994,125A as SEQ ID NO: 2, contains a Met in (matured) position 44, while in several published BPTI sequences there is an Asn in this position (see e.g., Uniprot P00974, residue 79 according to the full-length numbering). However, this difference does not influence the definition of the Kunitz domain from the point of view of the present invention. Thus, the first cysteine residue is residue 5 and the last cysteine is residue 55. An amino-acid sequence shall, for the purposes of the present invention, be deemed a Kunitz domain if it can be aligned, with three or fewer mismatches, to the sequence of SEQ ID NO: 1. An insertion or deletion of one residue shall count as one mismatch. In SEQ ID NO: 1, “x” matches any amino acid and “X” matches the types listed for that position. Disulfide bonds link at least two of: 5 to 55, 14 to 38, and 30 to 51. The number of disulfides may be reduced by one, but none of the standard cysteines shall be left unpaired. Thus, if one cysteine is changed, then a compensating cysteine is added in a suitable location or the matching cysteine is also replaced by a non-cysteine (the latter being generally preferred). For example, Drosophila funebris male accessory gland protease inhibitor has no cysteine at position 5, but has a cysteine at position −1 (just before position 1); presumably this forms a disulfide to Cys55. If Cys14 and Cys18 are replaced, the requirement of Gly12, (Gly or Ser)37, and Gly36 are dropped. From zero to many residues, including additional domains (including other Kunitz Domains), can be attached to either end of a Kunitz domain.

The general sequence of the Kunitz domains is as follows (SEQ

xxxxCxxxxxxGxCxxxxxxXXXxxxxxxCxxFxXXGCxXxxX
xXxxxxxCxxxCxxx

where:
x1 to x4, x58, x57, and x56 may be variable or absent,
x6 to x11, x13, x15 to x20, x24 to x29, x31 to x32, x34, x39, x41 to x42, x44, x46 to x50, x52 to x54 may be variable,
X21=Phe, Tyr, Trp,
X22=Tyr or Phe,
X23=Tyr or Phe,
X35=Tyr or Trp,
X36=Gly or Ser,
X40=Gly or Ala,
X43=Asn or Gly, and
X45=Phe or Tyr.

Here, “x” matches any amino acid and “X” matches the types listed for that position.

As outlined above, the present invention relates to a protein comprising an amino acid sequence of SEQ ID NO: 115, where the variable positions in the amino acid sequence of SEQ ID NO: 115 are limited in such a way that
x1 to x4, x58, x57, and x56 may be variable or absent,
x6 to x11, x13, x15 to x20, x24 to x29, x31 to x32, x34, x39, x41 to x42, x44, x46 to x50, x52 to x54 may be variable,
x21 is F, Y, or W,
x22 is Y or F,
x23 is Y or F,
x35 is Y or W,
x36 is G or S,
x40 is G or A,
x43 is N or G, and
x45 is F or Y;
characterized in that said protein
i) has an amino acid sequence segment of general formula Ih-mod, starting at position 12 and ending at position 19 of SEQ ID NO: 115:

(Ih-mod)
GX₁CX_{1V}X₂X₃X₄X₅,

where
X₁ is any of F, Y, L, P, Q, M, V, W, A, or T,
X_{1V} is R, or K,
X₂ is any of A, G, S, or T,
X₃ is any amino acid of the 17-set, where the 17-set comprises A, I, L, F, or Y,
X₄ is any of K, I, Q, R, H, S, F, M, N, L, or V, and
X₅ is any of R, V, I, K, M, Q, E, F, L, N, Y, D, S, H; and
ii) in position 34 said protein contains any amino acid of the 34-set, where the 34-set comprises Y, I, F, G, V and S;
and to salts, esters and pharmaceutically acceptable prodrugs of said protein.

The amino acid sequence of SEQ ID NO: 115 is the framework of the general Kunitz sequence (SEQ ID NO: 1), and the variable amino acids are defined as originally disclosed for the Kunitz domain in U.S. Pat. No. 5,994,125 as follows:
x1 to x4, x58, x57, and x56 may be variable or absent,
x6 to x11, x13, x15 to x20, x24 to x29, x31 to x32, x34, x39, x41 to x42, x44, x46 to x50, x52 to x54 may be variable,
x21 is F, Y, or W,
x22 is Y or F,
x23 is Y or F,
x35 is Y or W,
x36 is G or S,
x40 is G or A,
x43 is N or G, and
x45 is F or Y.

When a protein having an amino acid sequence of SEQ ID NO: 115 is referred to in the present description, the limitations defined in this paragraph are to be understood valid for said protein, if other interpretation is not explicitly disclosed.

Consequently, a protein of SEQ ID NO: 115 shows all characteristics of the Kunitz domain, i.e., it is a Kunitz domain protein. The proteins of the present invention are defined via SEQ ID NO: 115, where some variable parts of SEQ ID NO: 115 are restricted to arrive to the set of the proteins of the present invention.

One of these limitations concerns the eight-unit long P4-P4′ segment within the protein of SEQ ID NO: 115 located from position 12 to position 19 (see Table 100). This P4-P4′ segment can be any of the amino acid sequences defined above as the general formula Ih-mod. Position X₃ in Ih-mod is in position P2′ of the P4-P4′ segment, and is at position 17 of the amino acid sequence SEQ ID NO: 115. As this X₃ position has an outstanding importance compared to other positions of the P4-P4′ segment from the point of view of the present invention, the possible amino acids in this position (i.e., A, I, L, F, or Y) are mentioned as the 17-set throughout the present description for practical reasons.

The other limitation in SEQ ID NO: 115 concerns position 34, which is outside of the P4-P4′ segment into the C-terminal direction. According to the present invention, the possible amino acids in this position are Y, I, F, G, V and S. For practical reasons again, these amino acids are collectively named as the 34-set throughout the present description.

The basic concept behind the present invention is that if both above detailed limitations are applied to a protein with the amino acid sequence scaffold of SEQ ID NO: 115, we arrive to a set of proteins that effectively inhibit the human MASP-2 protein.

The protein sequence defined in this way (i.e., SEQ ID NO: 115 with said two essential limitations) taken on its own, or can be part of larger proteins. A protein having two or more Kunitz domains is also falling within the scope of the present invention, if at least one of the Kunitz domains fulfil the above outlined amino acid sequence criteria.

As obvious for a person skilled in the art, the claimed proteins can be in the form of salts, esters, or pharmaceutically acceptable prodrugs, which variations of the proteins fall also within the scope of the present invention.

The person skilled in the art understands that the sequence parts corresponding to the P4-P4′ segment defined by the general formula Ih-mod, and the defined sets (i.e., the essential 17-set and 34-set, and the optional 9-set, 39-set and 46-set) are the essence of the present invention. The person skilled in the art, however, also understands that other parts of the Kunitz domain protein of the present invention are also important for providing the necessary molecular environment for the effective spatial arrangement of the atoms of these elements. Further functions of the protein parts beyond said elements can be for example:
providing the necessary solubility of the protein in a drug product;
carrying other molecular objects, like e.g., labels, anchoring elements;
providing beneficial pharmacokinetic and pharmacodynamic properties for the drug product.

The person skilled in the art will understand that for ensuring these functions of the protein further molecular elements may be needed, like e.g., further amino acid sequence extensions on any of the end parts, modified amino acids, carbohydrate moieties, specific small molecular or biomolecular compounds, etc. Keeping the above mentioned essence of the present invention in mind, such kind of modified proteins are also within the scope of the present invention.

The present invention relates to Kunitz domain proteins and protein derivatives selectively inhibiting the human MASP-2 enzyme. By selective inhibition we understand first of all a selectivity over MASP-1, and enzymes of other proteolytic cascades in the blood such as the classical and alternative complement pathways and the extrinsic and intrinsic coagulation pathways. It will be obvious for a person skilled in the art that the protein environment of the amino acid sequence defined by SEQ ID NO: 115 may influence the successful inhibition.

Furthermore, the person skilled in the art will also understand that the modified Kunitz domain proteins of the present invention can be incorporated in an other protein such that said Kunitz domain protein maintains its MASP-2 inhibiting and lectin pathway blocking capacities in the resulting chimera protein. Keeping the above mentioned essence of invention in mind, such chimera proteins are also within the scope of the present invention. One such chimera protein is EVO24L (SEQ ID NO: 114) provided as a non-restrictive example (for details, see item E.3. in Example E below).

The scope of protection of the present invention also includes proteins into which elements ensuring detectability (e.g., fluorescent group, radioactive atom, etc.) are integrated. This kind of labelling is useful according to the state of the art in diagnostic methods, research works etc.

Furthermore, the scope of protection of the present invention also includes proteins that comprise further amino acids, amino acid sequences or protein domains at their N-terminal, C-terminal, or both ends, additional to what is defined by amino acid sequence scaffold of SEQ ID NO: 115 these further elements do not have a significant negative influence on the MASP-2 inhibitory activity of the original sequence. The aim of such further elements positioned at the ends may be to facilitate immobilisation, ensure the possibility of linking to other reagents, influence solubility, absorption, in vivo stability, pharmacokinetic and pharmacodynamic and other characteristics.

The present invention also relates to salts of the proteins of the present invention. In case said proteins are applied to the human body, e.g., within the framework of a pharmaceutical application, pharmaceutically acceptable salts are preferred. By the pharmaceutically acceptable salts are meant, which, during contact with the corresponding human tissues, do not result in an unnecessary degree of toxicity, irritation, allergic symptoms, or similar phenomena. As non-restrictive examples of acid addition salts, the following are mentioned: acetate, citrate, aspartate, benzoate, benzene sulphonate, butyrate, digluconate, hemisulphate, fumarate, hydrochloride, hydrobromide, hydroiodide, lactate, maleate, methane sulphonate, oxalate, propionate, succinate, tartrate, phosphate, glutamate. As non-restrictive examples of base addition salts, salts based on the following are mentioned: alkali metals and alkaline earth metals (lithium, potassium, sodium, calcium, magnesium, aluminium), quaternary ammonium salts, amine cations (methylamine, ethylamine, diethylamine, etc.).

Esters of the proteins according to the present invention involve all esters known by a person skilled in the art. In case said proteins are applied to the human body, e.g., within the framework of a pharmaceutical application, pharmaceutically acceptable esters are preferred. By the pharmaceutically acceptable esters are meant, which, during contact with the corresponding human tissues, do not result in an unnecessary degree of toxicity, irritation, allergic symptoms, or similar phenomena. It is within the general knowledge of a skilled person how to form esters using a surface functional group of a protein. These functional groups are typically alcoholic and carboxylic functional groups.

In respect of the present invention prodrugs are compounds that transform in vivo into a protein according to the present invention. Transformation can take place for example in the blood during enzymatic hydrolysis. In the prodrug form the compound is not active: it cannot fulfil its function. For example, if any of the amino acid residues of the inhibitory loop is covalently modified with a bulky compound, the loop cannot efficiently interact with any proteinases including MASP-2. If the chemical modification can be removed by a chemical reaction, e.g., hydrolysis, catalysed by a host enzyme, the prodrug will be transformed into the active drug. Protein modifications resulting in prodrugs are known for a person skilled in the art (Tobin 2014, Gou 2016).

According to a preferred embodiment, said protein is a human MASP-2 inhibitor with a K_{I} value equal to or lower than 100 nM. A compound is considered human MASP-2 inhibitor in the sense of the present invention if the K₁ value for the said interaction is determined to be equal to or lower than 100 nM. This corresponds to a level of inhibitory potency that can provide biologically relevant extent of MASP-2 inhibition. The person skilled in the art understands that a protein can be a human MASP-2 inhibitor only if it physically interacts with the human MASP-2 and thereby hinders binding of the substrates to human MASP-2 and/or hinders the function of the catalytic centre of the human MASP-2. We define inhibitory potency strictly through the measured value of the equilibrium inhibitory constant, i.e., the K_{I}. Said K_{I} value corresponding to the human MASP-2-inhibitor interaction is to be understood within the framework of the present invention as determined using an appropriate enzyme inhibitory kinetic assay that determines the concentration of uninhibited active human MASP-2 as a function of the concentration of the applied inhibitor at least as reliably as achieved by a modified version of the method of Empie and Laskowski (Empie, 1982) described in detail in (Szakács 2019). By active human MASP-2 we mean a protein having the UniProt ID 000187, which underwent proteolytic cleavage of the peptide bond after Arg-444, or any shorter, but catalytically equally active fragment of said protein. For details of the K_{I} measurement see Example F.2. below.

The present invention also relates to Kunitz domain proteins and protein derivatives which are sequentially analogous to the disclosed sequences of the present invention, i.e., those defined by the amino acid sequence scaffold of SEQ ID NO: 115 and the biological activity of which is also analogous when compared to the proteins of the present invention. A person skilled in the art finds it obvious that certain side chain modifications or amino acid replacements can be performed without altering the biological function of the protein in question. Such modifications may be based on the similarity of the amino acid side chains, for example on similarities in size, charge, hydrophobicity, hydrophilicity, etc. The aim of such changes may be to increase the stability of the protein against enzymatic decomposition or to improve certain pharmacokinetic or other parameters.

Similarity of two proteins can be defined in the percentage of similar or in the percentage of identical amino acids. To determine any of these percentage values, first the two amino acid sequences in question shall be aligned for the optimal comparison purposes. For example, gaps can be introduced in one or both of a first and a second amino acid sequence for optimal alignment and non-homologous sequences can be disregarded for comparison purposes, e.g., the Fc sequence part of the fusion protein according to the present invention. The optimal alignment is determined as the best score using the GAP program in the GCG software package with a Blosum 62 scoring matrix with a gap penalty of 12, a gap extend penalty of 4, and a frameshift gap penalty of 5. In the sense of the present invention, the general Kunitz domain sequence (SEQ ID NO: 1) is a good starting point for the alignment. When the amino acid sequences in question are aligned, the amino acid residues at corresponding amino acid positions are then compared. When a position in the first sequence is occupied by the same amino acid residue as the corresponding position in the second sequence, then the amino acids are identical at that position. The percent identity between the two sequences is a function of the number of identical positions shared by the sequences, taking into account the number of gaps, and the length of each gap, which need to be introduced for optimal alignment of the two sequences.

Importantly, within the sense of the present invention, sequence segments of the Ih-mod segment, and optionally positions 9, 34, 39, and 46 according to the Kunitz-numbering in SEQ ID NO: 1 are not counted when the percentage of similarity or identity are defined. Namely, only those aligned amino acid pairs of the two sequences are counted that fall out of the Ih-mod segment and are not in positions 9, 34, 39, and 46. For example, taking EVO215 (SEQ ID NO: 26) and the TFPI-D2 (SEQ ID NO: 116) proteins, the percentage of identity is calculated in the sense of the present invention as follows. In this calculation example, we take that embodiment into account, where only the P4-P4′ segment (i.e., Ih-mod) position 34 and position 9 are defined.

EVO215 (SEQ ID NO: 26):
KPDFCFLEND PGWCRAAKRR YFYNNQTKQC ERFGYGGCLG
NMNNFVTLEE CKNICEDG
TFPI-D2 (SEQ ID NO: 116):
KPDFCFLEED PGICRGYITR YFYNNQTKQC ERFKYGGCLG
NMNNFETLEE CKNICEDG

The P4-P4′ segment (i.e., Ih-mod), position 34 and position 9 are defined in this example, we do not calculate with them during the calculation of identity, they are shown underlined. Namely, out of the 58 amino acids, we calculate with 58-10, i.e., with 48 positions, i.e., those that are not underlined. There is one amino acid position, i.e., position 46, where there is a difference (see in bold typesetting). Consequently, out of the 48 positions (100%), in 47 positions there are identical amino acids, which results in 97.9% amino acid sequence identity. If we take another preferred embodiment, where apart from the essential positions defined by Ih-mod and position 34, also positions 9 and 46 are also defined, than position 46 would not count, and 47 positions out of the total 47 positions were identical resulting in a 100% sequence identity in the sense of the present invention.

One of the preferred Kunitz domains that proved to be useful in terms of the present invention is the TFPI-D2 protein, i.e., the second domain of the human Tissue Factor Pathway Inhibitor-1 protein (TFPI-1; UniProt ID P10646) (SEQ ID NO: 116). TFPI-D2 proved to be useful, when the modifications corresponding to the above mentioned two limitations were applied to its amino acid sequence, i.e., the sequence of the P4-P4′ segment shall be what is defined as Ih-mod, and the amino acid in position 34 shall be one of the amino acids of the 34-set. The skilled person will understand that beyond these limitations,—which are essential for the purpose of the present invention—certain flexibility can be allowed outside of the positions of these limitations regarding the amino acid composition of the Kunitz protein to remain a functional MASP-2 inhibitor. That is, some amino acids that are not part of the P4-P4′ segment and is not in position 34, may be substituted by an other amino acid, without risking a significant change in function, stability, etc. According to preferred embodiments of the present invention, positions 9, 39, and 46 are also defined by the present invention without leaving a room for replacement with similar amino acids. Consequently, also those proteins fall within the scope of the present invention that show a certain level of similarity with said parts of the TFPI-D2 protein (i.e., parts falling outside the P4-P4′ segment and position 34, and—according to preferred embodiments—fall also outside of positions 9, 39, and 46). Similarity in this context allows conservative substitutions of amino acid residues having similar physicochemical properties, if the protein said to show similarity is aligned to the TFPI-D2 protein (SEQ ID NO: 116). Within the context of the present invention, this similarity is at least 70%, or at least 80%, or at least 90%, or at least 95%, preferably it is at least 98%. Namely, those proteins fall within the scope of the present invention, that show at least 70%, or at least 80%, or at least 90%, or at least 95%, preferably at least 98% amino acid sequence similarity with a protein that comprise the claimed two limitations (i.e., the definition according to the general formula Ih-mod and the definition of the 34-set), and optionally the limitations in positions 9, 39, and 46, and said level of similarity can be determined for the sequence parts falling outside the parts affected by said limitations.

A subset of similar proteins can be determined by identity. In this sense, those proteins fall within the scope of the present invention that show at least 70%, or at least 80%, or at least 90%, or at least 95%, preferably at least 98% identity with a TFPI-D2 derived protein of the present invention, i.e., with a TFPI-D2 derived protein having said two limitations in the P4-P4′ segment and in position 34 (optionally also limitations in positions 9, 39, and 46), and beyond these positions, i.e., out of the P4-P4′ segment and position 34 (and optionally also out of positions 9, 39, and 46), the remaining sequence show at least 70%, or at least 80%, or at least 90%, or at least 95%, preferably 98% identity.

Proteins defined in this way by the degree of similarity or by a percentage of identity fall within the scope of the present invention, even if they are part of larger proteins. A protein having two or more Kunitz domains is also falling within the scope of the present invention, if at least one of the Kunitz domains fulfil the above outlined degree of similarity or percentage of identity criteria. It is obvious for a person skilled in the art to use protein alignment algorithms for the alignment of a larger protein with the claimed TFPI-D2 derived protein, taking the general Kunitz domain sequence (SEQ ID NO: 1) into consideration.

As defined above, the 17-set and the 34-set comprise five and six possible amino acids, respectively. Consequently, a total of “5×6”, i.e. thirty possible combinations are possible, if we do not count with the other variable positions in the protein. These thirty combinations are the following: (in x17/x34 format): A/Y, A/I, A/F, A/G, A/V, A/S, I/Y, I/I, I/F, I/G, I/V, I/S, L/Y, L/I, L/F, L/G, L/V, L/S, F/Y, F/I, F/F, F/G, F/V, F/S, Y/Y, Y/I, Y/F, Y/G, Y/V, Y/S. For example, the A/Y combination means that in the respective protein in position 17 (i.e., X₃ in the general sequence Ih-mod) there is an A (i.e., alanine), and in the exosite position 34 there is a Y (i.e., tyrosine). We found that all of said thirty pairwise combinations of the 17-set and the 34-set were positively selected for binding to MASP-2 through their characteristic combined hydrophobicity and cumulative side chain size ranges preferred by MASP-2. This is the reason why using the 17-set and 34-set members in the protein proved to be essential.

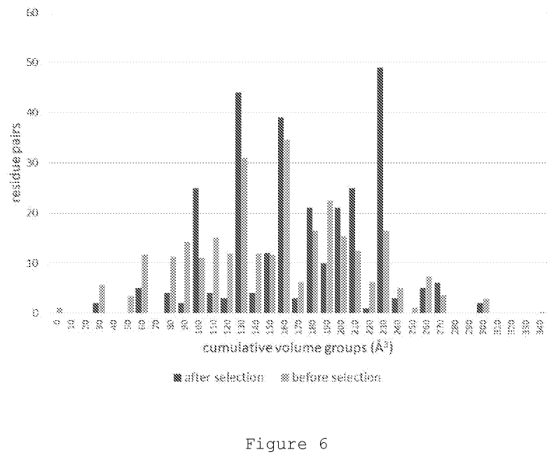

Out of the thirty possible and working pairwise x17/x34 combinations, the following twenty-five combinations are especially preferred: A/Y, A/I, A/F, A/V, I/Y, I/I, I/F, I/G, I/V, I/S, L/Y, L/I, L/F, L/G, L/V, L/S, F/I, F/G, F/V, F/S, Y/Y, Y/I, Y/G, Y/V, Y/S. These combinations are especially preferred, as their positive selection can be more reliably inferred from their observed frequencies, which clearly exceed the frequency value of the corresponding cumulative size range in the starting library prior to selection, as listed in Table 11 and plotted in FIG. 6. The other five members of the thirty x17/x34 combinations represent extremely small or extremely large cumulative side chain sizes. For pure combinatorial reason, the starting frequency of these sets is inherently low in the starting library and their positive selection usually requires a larger number of selection cycles.

As declared above, selecting a certain amino acid from the 17-set and an other one from the 34-set is essential to obtain a protein according to the objective of the present invention. However, during our research, further sites with certain amino acid possibilities were identified the use of which can enhance the efficacy of the MASP-2 inhibitors of the present invention. For these sites optional sets were defined during our research work. Some amino acids in these optional sets are those that can be found in natural Kunitz domain proteins (like e.g., in SEQ ID NO: 116), or can be such amino acids, that are not present in these positions in natural Kunitz domain proteins.

The 9-set defines optional amino acids for position 9, this 9-set comprises N or E. This means, that in a preferred embodiment of the present invention, N or E can be in position 9 of SEQ ID NO 200.

The 39-set defines optional amino acids for position 39, this 39-set comprises F or L. This means, that in a preferred embodiment of the present invention, F or L can be in position 39 of SEQ ID NO 200.

The 46-set defines optional amino acids for position 46, this 46-set comprises V or E. This means, that in a preferred embodiment of the present invention, V or E can be in position 46 of SEQ ID NO 200.

According to a more preferred embodiment, the present invention relates to proteins, that are selected from the following list. These sequences, i.e., from SEQ ID NO: 33 to SEQ ID NO: 52 and from SEQ ID NO: 54 to SEQ ID NO: 56 (twenty-three sequences), are general amino acid sequences, where x and X can be as defined above in relation to the general Kunitz domain sequence of SEQ ID NO: 1. Certain positions of these sequences, however, have exact amino acids. These exactly defined positions are either conserved residues, or residues obtained as a result of our research work. Bold and underlined positions show positions that are either essential or optional, however, defined positions in the sense of the present invention. E.g., in SEQ ID NO: 33, in position 9 an N is shown in bold and underlined, as defined above, the optional 9-set can be N or E; in position 34 an Y is shown in bold and underlined, as defined above, the essential 34-set can be Y, I, F, G, V and S; in position 39 an F is shown in bold and underlined, as defined above, the optional 39-set can be F or L; and in position 46 an V is shown in bold and underlined, as defined above, the optional 46-set can be V or E. The general sequence Ih-mod is GPCRALKR in SEQ ID NO 33, from which one can see that in position 17 there is an L, and the essential 17-set can be A, I, L, F, or Y. Each of these sequences from SEQ ID NO: 33 to SEQ ID NO: 52 and from SEQ ID NO: 54 to SEQ ID NO: 56 is a generalised form of a certain exact protein developed during our research work and proved to be efficient (see below). To each amino acid sequence this original protein is shown in brackets.

SEQ ID NO: 33 (generalised from EVO23):
xxxxCxxxNxxGPCRALKRxXXXxxxxxxCxxFYXXGCFXxxX
xXVxxxxCxxxCxxx
SEQ ID NO: 34 (generalised from EVO211):
xxxxCxxxNxxGWCRALKRxXXXxxxxxxCxxFGXXGCLXxxX
xXVxxxxCxxxCxxx
SEQ ID NO: 35 (generalised from EVO23a):
xxxxCxxxExxGPCRALKRxXXXxxxxxxCxxFYXXGCFXxxX
xXVxxxxCxxxCxxx
SEQ ID NO: 36 (generalised from EVO22a):
xxxxCxxxNxxGPCRAAKRxXXXxxxxxxCxxFYXXGCLXxxX
xXVxxxxCxxxCxxx
SEQ ID NO: 37 (generalised from EVO22):
xxxxCxxxNxxGPCRALKRxXXXxxxxxxCxxFYXXGCLXxxX
xXVxxxxCxxxCxxx
SEQ ID NO: 38 (generalised from EVO214):
xxxxCxxxNxxGPCRALKLxXXXxxxxxxCxxFGXXGCLXxxX
xXVxxxxCxxxCxxx
SEQ ID NO: 39 (generalised from EVO21b):
xxxxCxxxExxGPCRALKRxXXXxxxxxxCxxFGXXGCLXxxX
xXVxxxxCxxxCxxx
SEQ ID NO: 40 (generalised from EVO22d):
xxxxCxxxExxGPCRAAKRxXXXxxxxxxCxxFYXXGCFXxxX
xXVxxxxCxxxCxxx
SEQ ID NO: 41 (generalised from EVO25):
xxxxCxxxNxxGPCRALKRxXXXxxxxxxCxxFYXXGCFXxxX
xXExxxxCxxxCxxx
SEQ ID NO: 42 (generalised from EVO21):
xxxxCxxxNxxGPCRALKRxXXXxxxxxxCxxFGXXGCLXxxX
xXVxxxxCxxxCxxx
SEQ ID NO: 43 (generalised from EVO21c):
xxxxCxxxExxGPCRALKRxXXXxxxxxxCxxFGXXGCFXxxX
xXVxxxxCxxxCxxx
SEQ ID NO: 44 (generalised from EVO212):
xxxxCxxxNxxGLCRALKRxXXXxxxxxxCxxFGXXGCLXxxX
xXVxxxxCxxxCxxx
SEQ ID NO: 45 (generalised from EVO24):
xxxxCxxxNxxGPCRALKRxXXXxxxxxxCxxFYXXGCLXxxX
xXExxxxCxxxCxxx
SEQ ID NO: 46 (generalised from EVO21d):
xxxxCxxxExxGPCRALKRxXXXxxxxxxCxxFSXXGCFXxxX
xXVxxxxCxxxCxxx
SEQ ID NO: 47 (generalised from EVO214a):
xxxxCxxxExxGPCRALKLxXXXxxxxxxCxxFGXXGCFXxxX
xXVxxxxCxxxCxxx
SEQ ID NO: 48 (generalised from EVO222):
xxxxCxxxNxxGLCRAAAVxXXXxxxxxxCxxFYXXGCLXxxX
xXVxxxxCxxxCxxx
SEQ ID NO: 49 (generalised from EVO223):
xxxxCxxxNxxGPCRAAAVxXXXxxxxxxCxxFYXXGCLXxxX
xXVxxxxCxxxCxxx
SEQ ID NO: 50 (generalised from EVO211a):
xxxxCxxxExxGWCRALKRxXXXxxxxxxCxxFGXXGCFXxxX
xXVxxxxCxxxCxxx
SEQ ID NO: 51 (generalised from EVO221):
xxxxCxxxNxxGVCRAAAVxXXXxxxxxxCxxFYXXGCLXxxX
xXVxxxxCxxxCxxx
SEQ ID NO: 52 (generalised from EVO22b):
xxxxCxxxNxxGPCRALARxXXXxxxxxxCxxFYXXGCLXxxX
xXVxxxxCxxxCxxx
SEQ ID NO: 54 (generalised from EVO21a):
xxxxCxxxNxxGPCRAVKRxXXXxxxxxxCxxFGXXGCLXxxX
xXVxxxxCxxxCxxx
SEQ ID NO: 55 (generalised from EVO2c):
xxxxCxxxExxGPCRAVKRxXXXxxxxxxCxxFYXXGCLXxxX
xXExxxxCxxxCxxx
SEQ ID NO: 56 (generalised from EVO215):
xxxxCxxxNxxGWCRAAKRxXXXxxxxxxCxxFGXXGCLXxxX
xXVxxxxCxxxCxxx

According to a further preferred embodiment, the amino acid sequence of said protein has at least 70%, or at least 80%, or at least 90%, or at least 95% similarity, more preferably at least 98% similarity, even more preferably at least 70%, or at least 80%, or at least 90%, or at least 95% identity, most preferably 98% identity, or fully identical with any of the amino acid sequences set forth from SEQ ID NO: 3 to SEQ ID NO: 22 and from SEQ ID NO: 24 to SEQ ID NO: 32, with the proviso that the amino acid segment starting at position 12 and ending at position 19 has the sequence defined by the general formula Ih-mod. Percentage of similarity and identity shall be calculated within the framework of the present invention, as described above.

In the course of the studies leading to the present invention twenty-nine new MASP-2 inhibitors were developed, produced and tested, which are related to TFMI-2b of the invention disclosed in WO2018127719, hereafter referred to EVO2 (SEQ ID NO: 2). These proteins of the present invention were in part produced to infer sequence to activity algorithm relationships corresponding to improved MASP-2 inhibition, as well as to generate, as examples, highly improved MASP-2 inhibitors and lectin pathway inhibitors. The names, sequence identification number from SEQ ID NO: 3 to SEQ ID NO: 22 and from SEQ ID NO: 24 to SEQ ID NO: 32, in descending order of their human lectin pathway inhibitory potency, and sequences of these twenty-nine proteins of the present invention are listed in Table 1, together with the original EVO2 protein in the first place as a reference.

TABLE 1
Name, SEQ ID NO and sequence of proteins of the present invention in the order of
their relative human lectin pathway inhibitory efficiency compared to EVO2

| | SEQ | | Relative |
|---|---|---|---|
| Variant | ID NO: | Sequence | potency |
| EVO2 | 2 | KPDFCFLEEDPGPCRAVKRRYFYNNQTKQCERFKYGGCLGNMNNFETLEECKNICEDG | 1 |
| EVO23 | 3 | KPDFCFLENDPGPCRALKRRYFYNNQTKQCERFYYGGCFGNMNNFVTLEECKNICEDG | 47.1 |
| EVO211 | 4 | KPDFCFLENDPGWCRALKRRYFYNNQTKQCERFGYGGCLGNMNNFVTLEECKNICEDG | 44 |
| EVO23a | 5 | KPDFCFLEEDPGPCRALKRRYFYNNQTKQCERFYYGGCFGNMNNFVTLEECKNICEDG | 43.1 |
| EVO22a | 6 | KPDFCFLENDPGPCRAAKRRYFYNNQTKQCERFYYGGCLGNMNNFVTLEECKNICEDG | 37.1 |
| EVO22 | 7 | KPDFCFLENDPGPCRALKRRYFYNNQTKQCERFYYGGCLGNMNNFVTLEECKNICEDG | 33.4 |
| EVO214 | 8 | KPDFCFLENDPGPCRALKLRYFYNNQTKQCERFGYGGCLGNMNNFVTLEECKNICEDG | 32.4 |
| EVO21b | 9 | KPDFCFLEEDPGPCRALKRRYFYNNQTKQCERFGYGGCLGNMNNFVTLEECKNICEDG | 32.1 |
| EVO22d | 10 | KPDFCFLEEDPGPCRAAKRRYFYNNQTKQCERFYYGGCFGNMNNFVTLEECKNICEDG | 24.6 |
| EVO25 | 11 | KPDFCFLENDPGPCRALKRRYFYNNQTKQCERFYYGGCFGNMNNFETLEECKNICEDG | 24.5 |
| EVO21 | 12 | KPDFCFLENDPGPCRALKRRYFYNNQTKQCERFGYGGCLGNMNNFVTLEECKNICEDG | 24 |
| EVO21c | 13 | KPDFCFLEEDPGPCRALKRRYFYNNQTKQCERFGYGGCFGNMNNFVTLEECKNICEDG | 22.3 |
| EVO212 | 14 | KPDFCFLENDPGLCRALKRRYFYNNQTKQCERFGYGGCLGNMNNFVTLEECKNICEDG | 22.1 |
| EVO24 | 15 | KPDFCFLENDPGPCRALKRRYFYNNQTKQCERFYYGGCLGNMNNFETLEECKNICEDG | 18.1 |
| EVO21d | 16 | KPDFCFLEEDPGPCRALKRRYFYNNQTKQCERFSYGGCFGNMNNFVTLEECKNICEDG | 16.6 |
| EVO214a | 17 | KPDFCFLEEDPGPCRALKLRYFYNNQTKQCERFGYGGCFGNMNNFVTLEECKNICEDG | 15.2 |
| EVO222 | 18 | KPDFCFLENDPGLCRAAAVRYFYNNQTKQCERFYYGGCLGNMNNFVTLEECKNICEDG | 14.1 |
| EVO223 | 19 | KPDFCFLENDPGPCRAAAVRYFYNNQTKQCERFYYGGCLGNMNNFVTLEECKNICEDG | 14 |
| EVO211a | 20 | KPDFCFLEEDPGWCRALKRRYFYNNQTKQCERFGYGGCFGNMNNFVTLEECKNICEDG | 13.5 |
| EVO221 | 21 | KPDFCFLENDPGVCRAAAVRYFYNNQTKQCERFYYGGCLGNMNNFVTLEECKNICEDG | 10.8 |
| EVO22b | 22 | KPDFCFLENDPGPCRALARRYFYNNQTKQCERFYYGGCLGNMNNFVTLEECKNICEDG | 10 |
| EVO21a | 24 | KPDFCFLENDPGPCRAVKRRYFYNNQTKQCERFGYGGCLGNMNNFVTLEECKNICEDG | 6.4 |
| EVO2c | 25 | KPDFCFLEEDPGPCRAVKRRYFYNNQTKQCERFYYGGCLGNMNNFETLEECKNICEDG | 6.2 |
| EVO215 | 26 | KPDFCFLENDPGWCRAAKRRYFYNNQTKQCERFGYGGCLGNMNNFVTLEECKNICEDG | 6.1 |
| EVO213 | 27 | KPDFCFLENDPGPCRAAKRRYFYNNQTKQCERFGYGGCLGNMNNFVTLEECKNICEDG | 4.9 |
| EVO224 | 28 | KPDFCFLENDPGVCRALAVRYFYNNQTKQCERFYYGGCLGNMNNFVTLEECKNICEDG | 4.5 |
| EVO216 | 29 | KPDFCFLENDPGWCRAAKLRYFYNNQTKQCERFGYGGCLGNMNNFVTLEECKNICEDG | 4.5 |
| EVO2d | 30 | KPDFCFLEEDPGPCRALKRRYFYNNQTKQCERFKYGGCLGNMNNFETLEECKNICEDG | 2.3 |
| EVO2b | 31 | KPDFCFLEEDPGPCRAVKRRYFYNNQTKQCERFGYGGCLGNMNNFETLEECKNICEDG | 2.2 |
| EVO2a | 32 | KPDFCFLENDPGPCRAVKRRYFYNNQTKQCERFKYGGCLGNMNNFETLEECKNICEDG | 0.6 |

Out of these twenty-nine proteins of the present invention twenty-three are from 47-fold to 6-fold higher efficacy human lectin pathway inhibitors compared to EVO2. These twenty-three inhibitors from SEQ ID NO: 3 to SEQ ID NO: 22, and from SEQ ID NO: 24 to SEQ ID NO: 26 which are modified TFPI-D2 proteins, are especially preferred embodiments of the present invention, as increased inhibitor efficacy allows for reciprocally lower applied dose, which, in turn, lowers the burden of off-target-binding related side effects.

The present invention also relates to proteins, where the sequence determined by the scaffold of SEQ ID NO: 115, together with an antibody Fc-domain, form a fusion protein.

By using genetic engineering methods widely known by a person skilled in the art, two or more proteins, or domains of proteins can be fused together to generate a contiguous polypeptide, a fusion protein. In such a fusion protein the fused proteins or protein domains can retain their original structural and functional properties including their capacity to interact with their original binding partners that can be macromolecules, small molecules, ions or atoms. Therefore, in the structural context of a fusion protein construct, useful properties and functions of a peptide- or protein-based invention can be retained and combined with other useful functions provided by the fusion partners. For example, a fusion partner that binds to a tissue specific cell surface molecule can direct a peptide- or protein-based drug compound to said tissue, which provides increased specificity and efficacy.

The Fc domains of antibodies are often used as fusion partners for protein- or peptide-based drugs for several different purposes. Depending on their type and glycosylation state, Fc domains can bind to various soluble and cell surface proteins and thereby provide specific biological functionalities. For example, such binding partner can be the plasma protein C1q, which is the pattern recognition molecule of the classical complement pathway (for details, see above in the “Background of the invention” part). Binding of C1q to surface deposited Fc can trigger complement activation.

Other binding partners can be cell surface Fc receptor proteins that capture the Fc-fusion protein and trigger a specific cellular response. These interactions usually require a properly glycosylated Fc domain.

In the case of the present invention we used an IgG Fc fusion partner and produced the recombinant fusion protein in E. coli. As a result, the Fc domain was not glycosylated, which diminished its binding capacity towards most Fc-binding proteins, except the neonatal Fc receptor, FcRn.

The main function of FcRn is to dramatically extend the plasma half-life of immunoglobulins and albumins. As all plasma proteins, immunoglobulins and albumins are constantly taken up by endothelial and various white blood cells through pinocytosis. While most proteins are quickly degraded through the lysosomal pathway, immunoglobulins and albumins are captured by FcRn in the early endosome and are directed to the cell surface, where they are released back in the plasma.

According to a preferred embodiment, the fusion protein of the present invention is a protein showing at least 70%, or at least 80%, or at least 90%, or at least 95% similarity, more preferably at least 98% similarity, even more preferably at least 70%, or at least 80%, or at least 90%, or at least 95% identity, most preferably 98% identity, or is fully identical with SEQ ID NO: 114, i.e., EVO24L. Percentage of similarity and identity shall be calculated within the framework of the present invention, as described above. The protein EVO24L of the present invention proved to be very useful, as detailed in Example F.9.2. below. Briefly, the Fc fusion partner of EVO24L dramatically prolonged the presence of EVO24L in the rat blood circulation compared to EVO24 lacking an Fc fusing segment.

The present invention also relates to pharmaceutical preparations that contain at least one protein of the present invention, its pharmaceutically acceptable salt, pharmaceutically acceptable ester or pharmaceutically acceptable prodrug, and at least one additive. Said at least one protein is preferably selected from proteins comprising any of the amino acid sequences of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 54, SEQ ID NO: 55, and SEQ ID NO: 56; more preferably said protein is selected from the proteins comprising any of the amino acid sequences of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 24, SEQ ID NO: 25, and SEQ ID NO: 26, SEQ ID NO: 27, SEQ ID NO: 28, SEQ ID NO: 29, SEQ ID NO: 30, SEQ ID NO: 31, and SEQ ID NO: 32; most preferably said protein is selected from the proteins comprising any of the amino acid sequences of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 24, SEQ ID NO: 25, and SEQ ID NO: 26.

The proteins of the present invention can be used in pharmaceutical preparations suitable for the treatment of living organisms having MASP-2 proteins, like e.g., mammals, including humans. Pharmaceutical preparations for human use are especially preferred. Such pharmaceutical preparations contain apart from said protein at least one additive.

Additives are needed to reach the appropriate biological effect. Such preparations may be pharmaceutical preparations combined, for example, with matrices ensuring controlled active agent release, widely known by a person skilled in the art. Generally, matrices ensuring controlled active agent release are polymers that, when entering the appropriate tissue (e.g., blood plasma), decompose, for example in the course of enzymatic or acid-base hydrolysis (e.g., polylactide, polyglycolide). The additive is preferably a matrix ensuring controlled active agent release.

In the human pharmaceutical preparations according to the present invention other additives known in the state of the art can also be used, such as diluents, fillers, pH regulators, substances promoting dissolution, colouring additives, antioxidants, preservatives, isotonic agents, etc. These additives are known in the state of the art.

The pharmaceutical preparations according to the present invention are preferably in the form of infusions, tablets, powders, granules, suppositories, injections, syrups, inhalation and intranasal delivery systems.

Preferably, the human pharmaceutical preparations according to the invention can be entered in the organism via parenteral (intravenous, intramuscular, subcutaneous, intranasal, inhalation, etc.) administration. Taking this into consideration, preferable pharmaceutical compositions may be aqueous or non-aqueous solutions, dispersions, suspensions, emulsions, or solid (e.g., powdered) preparations, which can be transformed into one of the above fluids directly before use. In such fluids suitable vehicles, carriers, diluents or solvents may be, for example water, ethanol, different polyols (e.g., glycerol, propylene glycol, polyethylene glycols and similar substances), carboxymethyl cellulose, different (vegetable) oils, organic esters, and mixtures of all these substances.

The preferable formulations of the pharmaceutical preparations according to the invention include among others infusions, tablets, powders, granules, suppositories, injections, syrups, inhalation and intranasal delivery systems, etc. One of the preferred administration routes of proteins and peptides is the intranasal delivery to bypass the blood-brain barrier (Meredith 2015). Therefore, preferred preparations include intranasal delivery systems, like e.g., cyclodextrins, inhaled solutions, etc.

The administered dose depends on the type of the given disease, the patient's sex, age, weight, and on the severity of the disease. In the case of oral administration, the preferable daily dose may vary for example between 0.01 mg and 1 g, in the case of parenteral administration (e.g., a preparation administered intravenously) the preferable daily dose may vary for example between 0.001 mg and 1 g in respect of the active agent. A person skilled in the art finds it obvious that the dose to be selected depends very much on the molecular weight of the given protein used.

Furthermore, the pharmaceutical preparations can also be applied in liposomes or microcapsules known in the state of the art.

A nucleic acid encoding any of the proteins of the present invention are also falling within the scope of the present invention. The proteins according to the present invention can be entered into the target organism by state-of-the-art means of using natural or modified RNA or DNA molecules encoding the proteins according to the present invention. The protein will be generated through the actions of appropriate transcription and/or translation systems within the target organism. The delivery of such nucleic acids (e.g., in the form of an mRNA) can be done into a cell or cell system aiming at producing the protein of the present invention. Or the delivery can be made into the mammalian, preferably human body, e.g., in the form of an mRNA vaccine. Knowing the protein's amino acid sequence in question, a skilled person can determine the nucleic acid sequence of the corresponding DNA or RNA based on the well-known genetic code. The degeneracy of the genetic code makes it possible to tailor the DNA or RNA sequence to special needs.

Such nucleic acids can be incorporated into vectors for transfection purposes. Based on the sequence information of said nucleic acid, a skilled person is aware of designing, synthesizing and using such transfection vectors.

The present invention also relates to kits containing at least one protein, its salt or ester of the present invention, and manual for use or reference to such manual. These kits can be used for measuring and/or localising the MASP-2 enzyme. Such use may extend to competitive and non-competitive tests, radioimmunoassays, bioluminescent and chemiluminescent tests, fluorometric tests, enzyme-linked assays (e.g., ELISA), immunocytochemical assays, etc.

In accordance with the present invention, those kits are especially preferable, which are suitable for the examination of the potential inhibitors of the human MASP-2 enzyme, e.g., in competitive binding assays. With the help of such kits a potential inhibitor's ability of how much it can displace the protein according to the present invention from the MASP-2 enzyme can be measured. In order to detect it, the protein according to the present invention needs to be labelled in some way (e.g., incorporating a fluorescent group or radioactive atom, or other labelling means known according to the state of the art).

The kits according to the present invention may also contain other solutions, tools and starting substances needed for preparing solutions and reagents, and instruction manuals. Here, under instruction manual a simple reference to an online manual is also understood.

Further, the present invention relates to a screening procedure of compounds potentially inhibiting a MASP-2 enzyme, preferably the human MASP-2 enzyme, in the course of which i) a protein according to the present invention, its salt or ester, in a labelled form, is added to a solution containing said MASP-2 enzyme, preferably said human MASP-2 enzyme, then ii) the solution containing one or more compounds to be tested is added to it, and iii) the amount of the released labelled protein is measured. In the course of such a screening procedure the protein if the present invention is used in a labelled (fluorescent, radioactive, etc.) form in order to ensure detectability at a later point. The preparation containing such a protein is added to the solution containing the MASP-2 enzyme, or to a sample containing surface immobilized MASP-2 enzyme, in the course of which said labelled protein binds to the MASP-2 enzyme. Following the appropriate incubation period, a solution containing the compound/compounds to be tested is added to this preparation, which is generally followed by another incubation period. The compounds binding to the MASP-2 enzyme (if the tested compound binds to the surface of the MASP-2 enzyme partly or completely at the same site where the sequence according to the present invention is located, i.e., in a competitive manner, or somewhere else, but its binding alters the conformation of the MASP-2 enzyme in such a way that it loses its ability to bind to the protein, i.e., in a non-competitive manner) displace the labelled protein from the MASP-2 enzyme to the extent of their inhibiting ability. The concentration of the displaced proteins can be determined by using any method suitable for detecting the labelling (e.g., fluorescent or radioactive) used on the protein molecules of the present invention. The incubation periods, washing conditions, detection methods and other parameters can be optimised in a way known by the person skilled in the art. The screening procedure according to the invention can also be used in high-throughput screening (HTS) procedures, as is obvious for a person skilled in the art. The MASP-2 enzyme used in the screening procedure is preferably the human MASP-2 enzyme.

The present invention further relates to the use of said proteins, their pharmaceutically acceptable salts, esters or prodrugs, for the inhibition of MASP-2 protein, preferably human MASP-2 protein. It is apparent for a person skilled in the art, that an inhibitor (i.e., the proteins of the present invention) can be used in several applications where the inhibition of the target protein (i.e., the MASP-2) is useful. This use includes e.g., screening procedures, drug development processes (like e.g., in optimising lead compounds, use as reference compounds), identification of potential health conditions and diseases, assessing the relevance of mutations in the MASP-2 protein, etc.

The present invention relates also to the use of proteins, their pharmaceutically acceptable salts, esters or prodrugs, of the present invention in the production of a pharmaceutical preparation suitable for the treatment or prevention of diseases in the case of which the inhibition of the operation of the complement system has preferable effects. Said pharmaceutical preparation are used in organisms having MASP-2 enzyme, which can generally be mammals. However, most preferred pharmaceutical preparations of the present invention are human pharmaceutical preparations. The way of using proteins in the production of pharmaceutical preparations are well-known for a person skilled in the art.

Said diseases can be selected preferably from the following non-limiting groups:

(1) ischemia-reperfusion (IR) injuries (especially following recanalization after arterial occlusion due to thrombosis or other obstructive diseases), including those occurring after myocardial infarction (e.g., treated by percutaneous coronary interventions or thrombolysis), coronary bypass surgery, IR injury of the graft at organ transplantations, gastrointestinal IR injury, renal IR injury, post-ischemic brain injury, stroke, thrombosis affecting any region of the body; (2) inflammatory and autoimmune conditions with excess activation of the complement system, including autoimmune nephritis (including dense deposit disease, C3 glomerulonephritis), IgA nephropathy, membranous nephropathy, rheumatoid arthritis (RA), juvenile idiopathic arthritis, age-related macular degeneration, systemic lupus erythematosus (SLE), atypical hemolytic uremic syndrome (aHUS), thrombotic microangiopathy (TMA), post-infection hemolytic uremic syndrome (HUS), pseudo-allergy developing as a consequence of complement activation (CARPA), paroxysmal nocturnal hemoglobinuria (PNH), polytrauma, graft rejection after organ transplantation; venous thromboembolism (3) neurodegenerative diseases, preferably Alzheimer's disease, Huntington's disease, Parkinson's disease, multiple sclerosis and age-related macular degeneration; (4) complement overactivation caused by viral infection such as COVID-19 (SARS-CoV-2), acute respiratory distress syndrome (ARDS), complement associated microvascular injury and thrombosis due to severe COVID-19 infection.

The proteins according to the present invention are useful in the treatment of the above diseases.

The present invention relates also to a process for isolating the human MASP-2 enzyme, in the course of which i) a carrier with one or more immobilised proteins, their pharmaceutically acceptable salts, esters, of the present invention are contacted with a solution containing said human MASP-2 enzyme and ii) the preparation is washed. In the course of said process the proteins according to the present invention are immobilised and said immobilised proteins are contacted with the solution presumably containing the human MASP-2 enzyme. If this solution really contains the human MASP-2 enzyme, it will be anchored via the immobilised protein. This procedure can be suitable both for analytical and preparative purposes. The solution containing the human MASP-2 enzyme can be a pure protein solution, an extract purified to different extents, tissue preparation, etc.

### EXAMPLES

Below, the present invention is described in detail on the basis of examples, which, however, should not be regarded as examples to which the invention is restricted.

### Example A: The Concept Behind the Amino Acid Sequences

Conservation rules observed within the Kunitz family are described in Table 14 of U.S. Pat. No. 5,994,125A. These conservation rules were taken into consideration during our research work. The most important features are indicated for an illustration purpose in the following sequence of EVO2 (SEQ ID NO: 2) as follows (this amino acid sequence was described in WO2018127719 as SEQ ID NO: 14, and the protein named as TFMI-2b therein):

KPDFCFLEEDPGPCRAVKRRYFYNNQTKQCERFKYGGCLGNMN
NFETLEECKNICEDG

Bold typesetting highlights in the above SEQ ID NO: 2 fully conserved residues, while italic typesetting indicates positions that are represented by only 2-3 amino acid types within the Kunitz family. Positions that were randomized related to the invention described in WO2018127719, are underlined twice. Positions that were randomized related to the present invention are underlined either once or twice. Only non-conserved residues have been randomized.

For the better understanding, the above concept behind the present invention, as an example, for the sequence of the present invention we take GPCRALKR as one of the preferred sequences of general formula Ih-mod, N as one of the preferred amino acid of the 9-set, G as one of the preferred amino acid of the 34-set, L as one of the preferred amino acid of the 39-set and V as one of the preferred amino acid of the 46-set, and use the general Kunitz domain scaffold defined by SEQ ID NO: 115 as the host protein to get the protein of SEQ ID NO: 42:

xxxxCxxxNxxGPCRALKRxXXXxxxxxxCxxFGXXGCLXxxX
xXVxxxxCxxxCxxx

If the same combination of amino acids is introduced in the corresponding residue positions in EVO2 (SEQ ID NO: 2), we get SEQ ID NO: 12, which is the sequence of EVO21, a potent MASP-2 inhibitor of the present invention:

KPDFCFLENDPGPCRALKRxYFYNNQTKQCERFGYGGCLGNMN
NFVTLEECKNICEDG

where the double-underlined part indicates the sequence part of the general formula Ih-mod. Namely, SEQ ID NO: 42 is a general amino acid sequence that was generalised from the exact sequence of EVO21, namely from SEQ ID NO: 12.

Namely, preferred general amino acid sequences of the present invention, i.e., from SEQ ID NO: 33 to SEQ ID NO: 52 and from SEQ ID NO: 54 to SEQ ID NO: 56 can be obtained in a similar way from exact amino acid sequences of from SEQ ID NO: 3 to SEQ ID NO: 22 and from SEQ ID NO: 24 to SEQ ID NO: 26, as obvious for a person skilled in the art.

### Example B: Structure-Based Identification of EVO2 Residues that could be Evolved to Improve MASP-2 Inhibitory Potency

The protein EVO2 (described as TFMI-2b in WO2018127719), is a ˜7 nM inhibitor of both human and rat MASP-2. We solved the crystal structure of EVO2 in complex with rat MASP-2 (not yet published) and analysed which EVO2 positions outside the already evolves region carry residues that contact MASP-2, or could be evolved such that might become new contact position.

Five such positions in the EVO2 protein were identified that according to the Kunitz domain numbering are: x9 (an E), x10 (a D), x34 (a K), x39 (an L) and x46 (an E). MASP-2 surface loop nomenclature follows the serine protease loop nomenclature introduced in Perona and Craik (1997). The analysis led the following key observations: positions x9 and x10 can contact MASP-2 loop 3 from one side, why x39 can contact loop 3 from the opposite side. Position x34 has the potential to contact MASP-2 loop D. Position x46 is close to MASP-2 loop A. Positions x34 and x39 reside on the same loop that structurally supports the functional conformation of the canonical binding loop in part through the C14-C38 disulfide, which connects these loops.

This suggests that at least some positions of the canonical inhibitory loop and the supporting loop could influence the function of each other such that residue pairs could act synergistically.

### Example C: Three-Stage Directed Evolution Campaign Using Phage Display

This potential affinity source of affinity improvement was successfully investigated and utilized in a three-stage directed evolution campaign using phage display. The logic of the strategy is outlined below and the main conclusions are drawn here. To better understand how phage display works, the third of the three stages of the campaign will be described in detail in Example D.

### The Three-Stage Directed Evolution LED to New Insights and Significantly Improved MASP-2 Inhibitors

### Example C.1.: First Stage

In the first stage, we started from the EVO2 gene and simultaneously fully randomized the x9, x10, x34, x39 and x46 Kunitz positions, as well as the x13 position, which corresponds to the P3 position of the P4-P4′ segment. Moreover, at the x17 position, corresponding to the P2′ position of the P4-P4′ segment we applied a binary randomization allowing L and V.

Parallel selections were conducted on human and rat MASP-2 as described in detail in the “Phage display” Example D, in sections D1 and D2. Statistical analysis of the selected clones suggested that at position x9, an N instead of the original E amino acid could be slightly better, x10 evolved back to the wild type D, at the X13 P2′ position both human and rat MASP-2 preferred an L over V, at position x34 both human and rat MASP-2 selected against the wild type K, the human enzyme preferring a Y, while the rat enzyme preferring a G/N/Y in that order. At position x39 the human enzyme slightly preferred an F over the wild type L, but the rat enzyme strongly selected for an L over F. At position x46 there was no strong preference for any amino acid type but a V was the amino acid that both enzyme slightly selected for.

At position x13, i.e., the P3 position in the P4-P4′ segment, the original evolution campaign described in WO2018127719 resulted in an F/Y/L preference of human MASP-2 and a P/V/I preference for rat MASP-2. In the evolution campaign of the present invention the original preference of the rat enzyme was maintained, while the human enzyme allowed for many amino acid residues, slightly preferring for P/Y/A, indicating that some of the newly evolved positions act synergistically with the P3 position residue in inhibiting human MASP-2.

Based on these findings we designed and produced the following five new EVO2-based variants as recombinant proteins:

EVO21 (SEQ ID NO: 12):
KPDFCFLENDPGPCRALKRRYFYNNQTKQCERFGYGGCLGNMN
NFVTLEECKNICEDG
EVO22 (SEQ ID NO: 7):
KPDFCFLENDPGPCRALKRRYFYNNQTKQCERFYYGGCLGNMN
NFVTLEECKNICEDG
EVO23 (SEQ ID NO: 3):
KPDFCFLENDPGPCRALKRRYFYNNQTKQCERFYYGGCFGNMN
NFVTLEECKNICEDG
EVO24 (SEQ ID NO: 15):
KPDFCFLENDPGPCRALKRRYFYNNQTKQCERFYYGGCLGNMN
NFETLEECKNICEDG
EVO25 (SEQ ID NO: 11):
KPDFCFLENDPGPCRALKRRYFYNNQTKQCERFYYGGCFGNMN
NFETLEECKNICEDG

### Example C.2.: Second Stage

For the second stage we started from the variant EVO22 (SEQ ID NO: 7), in which, based on the finding of the first stage selection, x10 was kept as the wild type D, x39 was kept as the wild type L, while x9 was N, x34 was Y and x46 was V.

In this new amino acid sequence context we essentially repeated the same type of evolution described in WO2018127719 i.e., fully randomized the x13 (P3), x15 (P1), x16 (P1′), x17 (P2′) x18 (P3′) and x19 (P4′) positions. The library was, again, selected independently on both human and rat MASP-2.

According to our findings, at the P3 site human MASP-2 preferred W/L/M/F/Y, which resembles more the results of the first evolution campaign described in WO2018127719 than the pattern selected in the first stage of this campaign further supporting that some of the newly evolved positions act synergistically with the P3 position residue in inhibiting human MASP-2. In contrast, just like in the first stage campaign, P3 preference of the rat enzyme remained practically the same (V/P/I).

At the P1′ position a common preference for an A by both enzymes remained the same as described in WO2018127719.

At the P3′ and P4′ positions both enzymes seemed to be quite permissive without any significant preference pattern.

In contrast, at P2′ we identified a significant context dependence. In the original evolution campaign described in WO2018127719 in the context of K34, at P2′ (x17) position the human MASP-2 preferred V/A/I/L, while the rat enzyme preferred L/Y/I/M. In the first stage campaign where position x34 was fully randomized, the binary L/V randomization at P2′ (x17) resulted in an L preference. In the second stage, when Y34 was fixed, the pattern at x17 changed again. At this time, both enzymes preferred mostly a P2′ (x17) A, the preference for an L remained, while that for V disappeared suggesting that while the A17/Y34 and L17/Y34 combinations increase MASP-2 binding affinity, the V17/Y34 combination decreases it.

Based on these findings we produced four additional variants as recombinant proteins to test the roles of the P3 position and the functional coupling of the P2′ (x17) and x34 positions. The four variants are:

EVO221 (SEQ ID NO: 21):
KPDFCFLENDPGVCRAAAVRYFYNNQTKQCERFYYGGCLGNMN
NFVTLEECKNICEDG
EVO222 (SEQ ID NO: 18):
KPDFCFLENDPGLCRAAAVRYFYNNQTKQCERFYYGGCLGNMN
NFVTLEECKNICEDG
EVO223 (SEQ ID NO: 19):
KPDFCFLENDPGPCRAAAVRYFYNNQTKQCERFYYGGCLGNMN
NFVTLEECKNICEDG
EVO224 (SEQ ID NO: 28):
KPDFCFLENDPGVCRALAVRYFYNNQTKQCERFYYGGCLGNMN
NFVTLEECKNICEDG

### Example C.3.: Testing the Variants from the First and Second Stages

The five new variants from the first stage and the four new variants from the second stage were tested for human and rat MASP-2 binding affinity in surface plasmon resonance experiments and their human and rat lectin pathway inhibitory efficacy was determined in serum tests. Based on the obtained results summarized in Table 15 for SPR data and Table 16 for lectin pathway inhibitory data, thirteen additional proteins of the present invention were designed and produced as recombinant proteins and tested in the mentioned functional assays. These data are summarized in Table 15 and Table 16, described in Example F.3, F.4.1. and F.5.1. The thirteen new variants are as follows:

EVO214 (SEQ ID NO: 8):
KPDFCFLENDPGPCRALKLRYFYNNQTKQCERFGYGGCLGNMN
NFVTLEECKNICEDG
EVO211 (SEQ ID NO: 4):
KPDFCFLENDPGWCRALKRRYFYNNQTKQCERFGYGGCLGNMN
NFVTLEECKNICEDG
EVO22a (SEQ ID NO: 6):
KPDFCFLENDPGPCRAAKRRYFYNNQTKQCERFYYGGCLGNMN
NFVTLEECKNICEDG
EVO212 (SEQ ID NO: 14):
KPDFCFLENDPGLCRALKRRYFYNNQTKQCERFGYGGCLGNMN
NFVTLEECKNICEDG
EVO22b (SEQ ID NO: 22):
KPDFCFLENDPGPCRALARRYFYNNQTKQCERFYYGGCLGNMN
NFVTLEECKNICEDG
EVO215 (SEQ ID NO: 26):
KPDFCFLENDPGWCRAAKRRYFYNNQTKQCERFGYGGCLGNMN
NFVTLEECKNICEDG
EVO2a (SEQ ID NO: 32):
KPDFCFLENDPGPCRAVKRRYFYNNQTKQCERFKYGGCLGNMN
NFETLEECKNICEDG
EVO2b (SEQ ID NO: 31):
KPDFCFLEEDPGPCRAVKRRYFYNNQTKQCERFGYGGCLGNMN
NFETLEECKNICEDG
EVO2c (SEQ ID NO: 25):
KPDFCFLEEDPGPCRAVKRRYFYNNQTKQCERFYYGGCLGNMN
NFETLEECKNICEDG
EVO2d (SEQ ID NO: 30):
KPDFCFLEEDPGPCRALKRRYFYNNQTKQCERFKYGGCLGNMN
NFETLEECKNICEDG
EVO21a (SEQ ID NO: 24):
KPDFCFLENDPGPCRAVKRRYFYNNQTKQCERFGYGGCLGNMN
NFVTLEECKNICEDG
EVO213 (SEQ ID NO: 27):
KPDFCFLENDPGPCRAAKRRYFYNNQTKQCERFGYGGCLGNMN
NFVTLEECKNICEDG
EVO216 (SEQ ID NO: 29):
KPDFCFLENDPGWCRAAKLRYFYNNQTKQCERFGYGGCLGNMN
NFVTLEECKNICEDG

### Example C.4.: Third Stage

In the third directed evolution stage using phage display we allowed all possible combinations of the twenty amino acids at the P3 (x13), the P2′ (x17) and the x34 positions and allowed for the occurrence of R/K/T amino acids at the P1 position of the initial library. This allowed for testing if K is a better P1 residue than R in some sequential context. The threonine was allowed in order to see whether it is completely eliminated upon binding selection.

These randomisations were carried out in the context of a modified EVO214 sequence:

EVO214a (SEQ ID NO: 17):
KPDFCFLEEDPGPCRALKLRYFYNNQTKQCERFGYGGCFGNMNNFVTLE
ECKNICEDG

that contains an E at x9, F at x39 and V at x46, the latter two offering slightly higher affinity for human MASP-2.

The library was produced and selected independently for binding to human and rat MASP-2 as described in detail below. The selected clones were tested for binding to both rat and human. Clones that were able to bind to the human MASP-2 enzyme regardless of which enzyme they were selected on were analysed and the optimal x17/x34 amino acid combinations were determined.

Based on this new information seven additional new variants were designed, produced as recombinant proteins and tested for human and rat lectin pathway inhibitory efficacy in serum tests. The obtained results are summarized in Table 16 in Example F, section F.4.1.

These variants are as follows:

EVO21b (SEQ ID NO: 9):
KPDFCFLEEDPGPCRALKRRYFYNNQTKQCERFGYGGCLGNMNNFVTLE
ECKNICEDG
EVO21c (SEQ ID NO: 13):
KPDFCFLEEDPGPCRALKRRYFYNNQTKQCERFGYGGCFGNMNNFVTLE
ECKNICEDG
EVO21d (SEQ ID NO: 16):
KPDFCFLEEDPGPCRALKRRYFYNNQTKQCERFSYGGCFGNMNNFVTLE
ECKNICEDG
EVO214a (SEQ ID NO: 17):
KPDFCFLEEDPGPCRALKLRYFYNNQTKQCERFGYGGCFGNMNNFVTLE
ECKNICEDG
EVO211a (SEQ ID NO: 20):
KPDFCFLEEDPGWCRALKRRYFYNNQTKQCERFGYGGCFGNMNNFVTLE
ECKNICEDG
EVO22d (SEQ ID NO: 10):
KPDFCFLEEDPGPCRAAKRRYFYNNQTKQCERFYYGGCFGNMNNFVTLE
ECKNICEDG
EVO23a (SEQ ID NO: 5):
KPDFCFLEEDPGPCRALKRRYFYNNQTKQCERFYYGGCFGNMNNFVTLE
ECKNICEDG

In all, based on the sequence features of clones selected in the three-stage directed evolution campaign, as well as the functional properties of the altogether twenty-nine new proteins of the present invention, we surprisingly found that Kunitz domain protein based compounds having the sequence of the modified general formula Ih-mod, combined with the 34-set, further optionally combined with the 9-set, the 39-set and/or the 46-set meet the objective of the present invention, i.e., they are significantly more efficient inhibitors of the human MASP-2 enzyme than those described in WO2018127719.

### Example D: Phage Display

The proteins according to the present invention were developed using the phage display method described below.

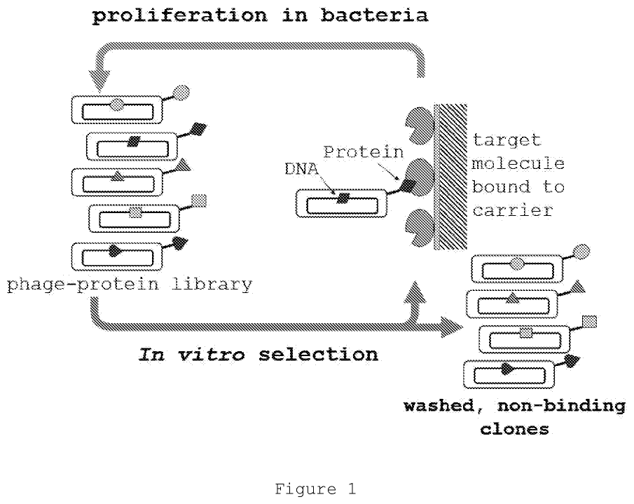

The phage display is suitable for the realisation of directed in vitro evolution of proteins and peptides. The main steps of the state-of-the-art procedure (Smith 1985) is depicted in FIG. 1. In the course of this procedure the gene of the protein involved in evolution is linked to a bacteriophage envelope protein gene. If phage or bacteriophage is mentioned throughout the present description, class I filamentous phage e.g., M13 bacteriophage is meant. In this way, when the bacteriophage is created, a fusion protein is produced which becomes incorporated into the surface of the phage. The phage particle carries the gene of the foreign protein inside, while on its surface it displays the foreign protein. The protein and its gene are physically linked via the phage. For directed protein evolution, we change the codons of the gene coding it, carefully determined by us. Numerous codons can be changed at the same time using combinatorial mutagenesis based on a mixture of synthetic oligonucleotides. The position of the mutations and variability per position is determined at the same time.

After creating a DNA library containing typically several billions of variants and entering it into bacteria, the phage protein library is created. Each phage displays only one type of protein variant and carries only the gene of this variant. The individual variants can be separated from each other using methods analogous to affinity chromatography, on the basis of their ability to bind to a given target molecule chosen by the researcher. Generally, the target molecule is linked or bound to a surface and serves as the stationary phase of the affinity chromatography process. At the same time, as opposed to simple protein affinity chromatography, the so-called protein-phages that were selected in this way and carry target-binding variants of the displayed protein have two important characteristic features. On the one part, they are able to multiply in E. coli cells, on the other part these particles also display the selected variants of the displayed protein and carry the coding genes wrapped in the phage particles.

During evolution, instead of examining individual mutants, in actual fact billions of experiments are performed simultaneously. Binding variants are multiplied, and after several cycles of selection-multiplication a population rich in functional variants is obtained. From this population, individual phage clones displaying one selected variant of evolved protein are examined in functional tests. The phage protein variants found appropriate during the tests are identified by sequencing the physically linked gene. Besides the individual measurements, through the sequence analysis of an appropriately large number of function-selected clones it is also revealed what amino acid sequences enable fulfilling the function. In this way, a database based on real experiments is prepared which makes it possible to elaborate a sequence-function algorithm. The variants found the best on this basis are also produced as independent proteins, and these are examined in more accurate further tests.

We developed the vectors suitable for phage display from the vectors available in commercial distribution, they will be described later.

### Example D.1.: The Starting Molecules of Phage Display Based Protein Evolution

As mentioned above, the present invention was developed in three consecutive directed evolution stages. The first stage started from the modified TFPI-D2 protein, where TFPI-D2 refers to the second domain (SEQ ID NO: 116, residues 121-178) of the human Tissue Factor Pathway Inhibitor-1 protein (TFPI-1; UniProt ID P10646). This modified TFPI-D2 was the TFMI-2b of the invention described in WO2018127719, which is referred here as EVO2 (SEQ ID NO: 2). In the first stage we fully randomized five previously non-tested, and based on the EVO2: rat MASP-2 structure, potential human MASP-2 contacting positions according to the general Kunitz domain sequence (SEQ ID NO: 1), x9, x10, x34, x39 and x46, and also fully randomized x13, which is the P3 position of the canonical inhibitory loop. Moreover, we performed a binary randomization at x17, the P2′ position of the canonical inhibitory loop (i.e., the P4-P4′ segment). The library was selected independently on both human and rat MASP-2. Based on this selection, we designed and produced as recombinant protein five proteins of the present invention, and out of these, EVO22 (SEQ ID NO: 7) served as the starting variant for the second stage directed evolution. At sequences outside the canonical inhibitory loop EVO22 differs from EVO2 by having N9/L39/V46 residues.

For the second stage evolution we used this new amino acid sequence context and essentially repeated the same type of canonical inhibitory loop evolution described in WO2018127719, i.e., fully randomized the x13 (P3), x15 (P1), x16 (P1′), x17 (P2′), x18 (P3′) and x19 (P4′) positions. The library was, again, selected independently on both human and rat MASP-2.

Based on the obtained sequence patterns of the selected clones we designed a new protein, EVO214a (SEQ ID NO: 17), which served as the starting sequence for the third stage evolution that particularly focused on the synergistic interplay of positions x17 and x34.

### Example D.2.: Creating a Library

The phagemid vector construct applied in the invention described in WO2018127719 was used in all three stages of the directed evolution leading to the present invention. That vector displays the Kunitz domain inhibitor fused to the M13 phage p8 protein in a monovalent fashion, i.e., no more than a single copy of the inhibitor is displayed on the phage particle. This is essential for selecting high-affinity binding molecules as higher copy number could lead to avidity, i.e., simultaneous binding of the phage particle to the surface through many individual inhibitor/target molecule pairs.

In the system used by us, the phage-TFPI-D2 variant library was created through a glycine-serine linker as an N-terminal fusion of the p8 main envelope protein.

Preceding the N-terminus of the TFPI-D2 library members, we also inserted a linear epitope tag, so-called “Flag-tag”, recognisable by monoclonal antibodies, using an appropriate distance-keeping peptide linker. This served the purpose of demonstrating successful display of any library member on phage, even those that do not bind to the target proteinase, MASP-2 (or any other given proteinase).

Through the examples below we show how the three phage libraries were created for the three-stage evolution (Example D.2.1.). To avoid unnecessary repetition of methodology description, we describe the phage selection only for the third stage evolution part (Example D.2.2.), but introduce the results for all three evolution stages (Example D.2.3.). In Example D.2.4., the method of the heterologous expression of the inhibitors is described, while in Example D.2.5., we describe how the inhibitors related to the present invention were tested for quality and efficacy.

All oligonucleotides needed for phage display based protein evolution are organized in Table 2.

TABLE 2
Oligonucleotides used for phage display based protein evolution
Name, SEQ ID NO and sequence of oligos and synthetic genes used for phage display evolution

| | | |
|---|---|---|
| EVO2-stage-1-stop | SEQ ID NO: 57 | GGGTCCGGAGGCTCGGGCAAACCGGACTTCTGCTTCCTGGAATAATAACCGGGTTAATGCCGTGCGTAAAAACGTCGTTACTTCTA |
| | | 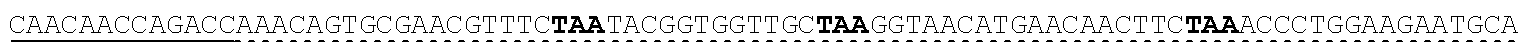 |
| | | 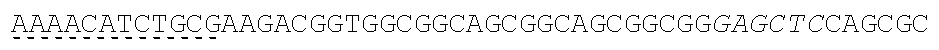 |
| EVO2-stage-1-lib-1 | SEQ ID NO: 58 | CGGGCAAACCGGACTTCTGCTTCCTGGAANNKNNKCCGGGTNNKTGCCGTGCGSTGAAACGTCGTTACTTCTACAACAACCAGAC |
| EVO2-stage-1-lib-2 | SEQ ID NO: 59 | CAAACAGTGCGAACGTTTCNNKTACGGTGGTTGCNNKGGTAACATGAACAACTTCNNKACCCTGGAAGAATGCAAAAACATCTGCG |
| EVO2-stage-2-stop | SEQ ID NO: 60 | 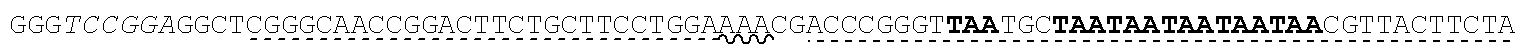 |
| | | 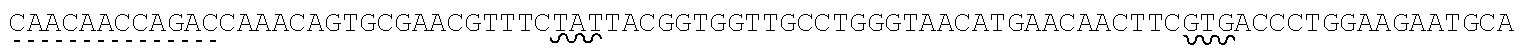 |
| | | AAAACATCTGCGAAGACGGTGGCGGCAGCGGCGGCAGCGGCGGGAGCTCCAGCGC |
| EVO2-stage-2-lib | SEQ ID NO: 61 | CCTGGAAAACGACCCGGGTNNKTGCNNKNNKNNKNNKNNKCGTTACTTCTACAACAACCAGACC |
| EVO2-stage-3-stop-1 | SEQ ID NO: 62 | CGGGCAAACCGGACTTCTGCTTCCTGGAAGAAGACCCGGGTTAATGCTAAGCGTAAAAACTTCGTTACTTCTACAACAACCAGAC |
| EVO2-stage-3-stop-2 | SEQ ID NO: 63 | CAAACAGTGCGAACGTTTCTAATACGGTGGTTGCTTCGGTAACATGAACAACTTCGTAACCCTGGAAGAATGCAAAAACATCTGCG |
| EVO214a-STOP | SEQ ID NO: 64 | GGGTCCGGAGGCTCGGGCAAACCGGACTTCTGCTTCCTGGAAGAAGACCCGGGTTAATGCTAAGCGTAAAAACGTCGTTACTTCTA |
| | | 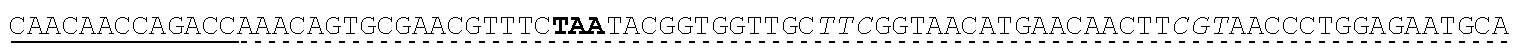 |
| | | 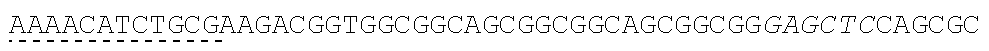 |
| EVO2-stage-3-lib-1 | SEQ ID NO: 65 | CGGGCAAACCGGACTTCTGCTTCCTGGAAGAAGACCCGGGTNNKTGCAVAGCGNNKAAACTTCGTTACTTCTACAACAACCAGAC |
| EVO2-stage-3-lib-2 | SEQ ID NO: 66 | CAAACAGTGCGAACGTTTCNNKTACGGTGGTTGCTTCGGTAACATGAACAACTTCGTAACCCTGG |

### Example D.2.1: Creating the Phage Library 1

### D.2.1.1. Constructing the Phage Display Vectors

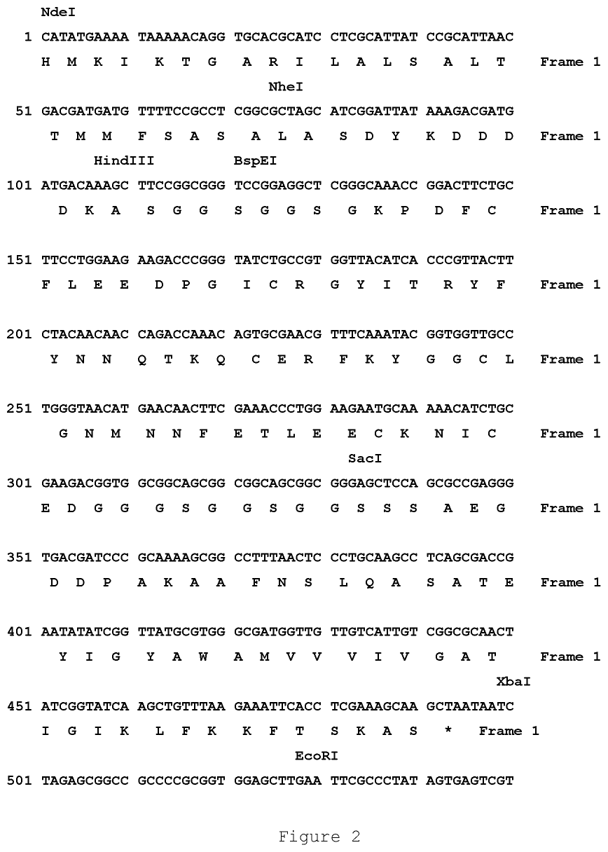

We started from the vector originally introduced and described in detail in WO2018127719. The vector contains the codon optimized version of the coding DNA of TFPI-D2 such, that the protein is flanked by Ser/Gly linkers on both termini and is displayed as a p8 coat protein fusion on the surface of bacteriophage M13. Moreover, the construct also provides the displayed protein with an N-terminal FLAG-tag for easy assessment of display efficiency. The coding DNA is located between Kpn2I (BspEI) and SacI sites as illustrated in FIG. 2, which depicts the DNA sequence (SEQ ID NO: 117) and amino acid sequence (SEQ ID NO: 118) of the fusion gene made to display TFPI-D2 on the surface of the M13 bacteriophage.

The different functional parts of the fusion protein as follows (numbering is according to nucleotides in FIG. 2): from position 4 to 81 is the malE periplasmic signal sequence, from position 85 to 108 is the FLAG-tag, from position 111 to 135 is a first Ser-Gly linker, from position 136 to 309 is the TFPI-D2 domain, from position 310 to 342 is a second Ser-Gly linker, and from position 343 to 492 is the major coat protein (p8) of the M13 phage. The restriction endonuclease cleavage sites used during the construction of the fusion gene are shown in FIG. 2 above the DNA sequence of their cleavage sites. Residues that were targeted by directed evolution related to the prior invention are shown from nucleotide position 172 to 174 and from 178 to 192.

For the first stage evolution we designed the following synthetic DNA named EVO2-stage-1-stop (SEQ ID NO: 57):

GGGTCCGGAGGCTCGGGCAAACCGGACTTCTGCTTCCTGGAATAATAACCGGGTTAATG

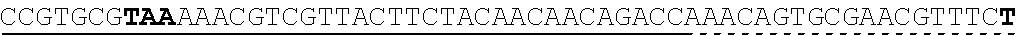

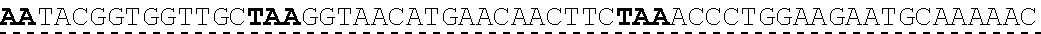

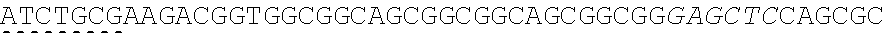

SEQ ID NO: 57 contains the codon optimized and stop codon containing coding DNA of a thereby modified EVO2 coding DNA that replaces the TFPI-D2 gene between BspEI (TCCGGA, italic) and SacI (GAGCTC, italic) sites. The EVO2 coding DNA is modified such that codon positions corresponding to the x9, x10, x13, x17, x34, x39 and x46 amino acid positions carry TAA stop codons. Continuous and staggered lines indicate the segments that in reverse complement form served as template regions for library mutagenesis oligonucleotides described below.

The following library mutagenesis oligonucleotides were used:

EVO2-stage-1-lib-1 (85-mer)
(SEQ ID NO: 58)
CGGGCAAACCGGACTTCTGCTTCCTGGAANNKNNKCCGGGTNNKTGCCG
TGCGSTGAAACGTCGTTACTTCTACAACAACCAGAC
and
EVO2-stage-1-lib-2 (86-mer)
(SEQ ID NO: 59)
CAAACAGTGCGAACGTTTCNNKTACGGTGGTTGCNNKGGTAACATGAAC
AACTTCNNKACCCTGGAAGAATGCAAAAACATCTGCG

where degenerate codons replacing the stop codons are indicated as bold.

For the second stage evolution we designed the following synthetic DNA: EVO2-stage-2-stop (SEQ ID NO: 60):

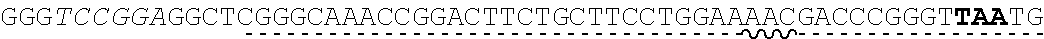

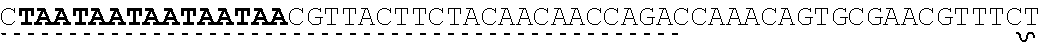

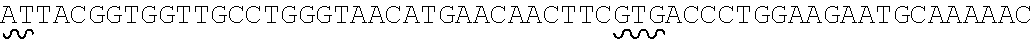

ATCTGCGAAGACGGTGGCGGCAGCGGCGGCAGCGGCGGGAGCTCCAGCGC

where bold indicates the stop codons replacing amino acid codons of positions x13, x15, x16, x17, x18, x19, waving line indicates exosite positions with newly introduced mutation at x9 (N9), x34 (Y34) and x46 (V46) and dotted line indicates the segment that served as template region for the library mutagenesis oligonucleotide described below.

The following library mutagenesis oligonucleotide was used:

EVO2-stage-2-lib
(SEQ ID NO: 61)
CCTGGAAAACGACCCGGGTNNKTGCNNKNNKNNKNNKNNKCGTTACTTC
TACAACAACCAGACC

where degenerate codons replacing the stop codons are indicated as bold.

For the third stage evolution we used the same synthetic DNA as a template that was used for the first stage evolution, EVO2-stage-1-stop (SEQ ID NO: 57):

GGGTCCGGAGGCTCGGGCAAACCGGACTTCTGCTTCCTGGAATAATAACCGGGTTAATG

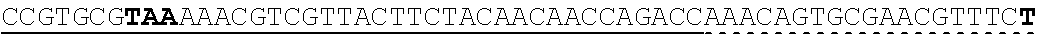

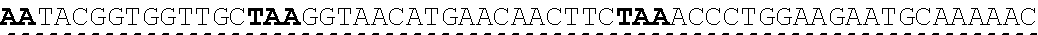

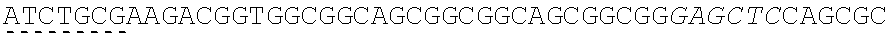

Continuous and staggered lines indicate the segments that in their reverse complement form served as template regions for mutagenesis oligonucleotides (SEQ ID NO: 62 and SEQ ID NO: 63) that were used to generate the proper stop template for the subsequent library mutagenesis.

Stop template producing oligonucleotide EVO2-stage-3-stop-1 (SEQ ID NO: 62):

CGGGCAAACCGGACTTCTGCTTCCTGGAAGAAGACCCGGGTTAATGCTA
AGCGTAAAAACTTCGTTACTTCTACAACAACCAGAC

Stop template producing oligonucleotide EVO2-stage-3-stop-2 (SEQ ID NO: 63):

CAAACAGTGCGAACGTTTCTAATACGGTGGTTGCTTCGGTAACATGAAC
AACTTCGTAACCCTGGAAGAATGCAAAAACATCTGCG

When these oligonucleotides were used together as mutagenesis primers on a template DNA containing the reverse complementary sequence of SEQ ID NO: 57, they created the coding DNA of a modified version of the original EVO214a (SEQ ID NO: 17) such that it codes for an E at x9, an F at x39 and a V at x46, indicated in italic, the latter two offering slightly higher affinity for human MASP-2. However, the resulting gene will have stop codons, indicated with bold, at the P3 (x13), the P1 (x15), P2′ (x17) and the x34 coding codon positions.

The resulted gene sequence shown below is EVO214a-STOP, (SEQ ID NO: 64):

GGGTCCGGAGGCTCGGGCAAACCGGACTTCTGCTTCCTGGAAGAAGACCCGGGTTAATG

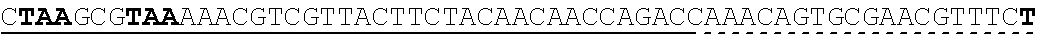

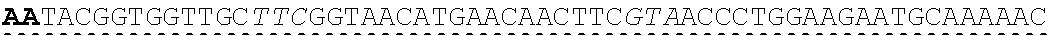

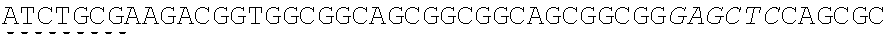

Continuous and staggered lines indicate the segments that in reverse complement form served as template regions for library mutagenesis oligonucleotides described below.

Library mutagenesis oligonucleotide EVO2-stage-3-lib-1, (SEQ ID NO: 65):

CGGGCAAACCGGACTTCTGCTTCCTGGAAGAAGACCCGGGTNNKTGCAV
AGCGNNKAAACTTCGTTACTTCTACAACAACCAGAC

Library mutagenesis oligonucleotide EVO2-stage-3-lib-2 (SEQ ID NO: 66):

CAAACAGTGCGAACGTTTCNNKTACGGTGGTTGCTTCGGTAACATGAAC
AACTTCGTAACCCTGG

In the library mutagenesis oligonucleotides bold indicates degenerate codons NNK, which codes the complete set of the twenty amino acids (at positions x13 (the P3), x17 (the P2′) and x34), and AVA, which represents the codon set AAA, AGA and ACA and thereby codes for amino acids K, R, T (at x15, which is the P1 position).

These oligonucleotides together create the coding DNA of a modified randomized version of EVO214a (SEQ ID NO: 17) such that allowed for all possible combinations of the twenty natural amino acids at the P3 (x13), the P2′ (x17) and the x34 positions and allowed for the occurrence of R/K/T amino acids at the P1 position of the initial library. This allowed for testing if K is a better P1 residue than R in some sequential contexts. The threonine was allowed in order to see whether it is completely eliminated upon binding selection, i.e., the selections works.

Now that essential DNA constructs and mutagenesis oligonucleotides were introduced, we can start to provide examples on how these tools are used for phage display based directed evolution. Although the present invention is based on three stages of directed evolution, in terms of technical realization all three stages applied the same methodologies in the same order. In order to avoid unnecessary repetitions, we explain these methodologies and steps in detail, always in relation to one of the three evolution stages, in order to avoid unnecessary repetitions of the same technology descriptions. In the following sections we show directed evolution steps of the third stage evolution campaign.

(Example No. D.2.1.2. is excluded intentionally.)

### D.2.1.3. Creating the DNA Library

### D.2.1.3.1. Creating Stop Mutant Phagemid by Kunkel Mutagenesis

D.2.1.3.1.1. Transformation of CJ 236 E. coli Strain

### Starting DNA Solution:

1 μl (˜100 ng) pTFPI-D2-pro-lib phagemid
4 μl 5×KCM (0.5 M KCl, 0.15 M CaCl₂), 0.25 M MgCl₂)
15 μl distilled water

The DNA solution was cooled on ice.

We added 20 μl CJ236 cells (NEB) to the DNA solution and the sample was incubated for 20 minutes on ice. Then we left the cells alone for 10 minutes at room temperature, and after adding 200 μl LB medium we shook it for 30 minutes at 37° C. The cells were spread onto an LB-agar+ampicillin (100 μg/ml) plate and grown overnight at 37° C.

### D.2.1.3.1.2. Production and Isolation of Uracil-Containing Phage

From a separate colony, cells were inoculated in 2 ml 2YT/ampicillin (100 μg/ml), chloramphenicol (5 μg/ml) medium and grown overnight, shaken at 37° C. On the following day 30 μl culture was inoculated in 3 ml medium of the same composition. As soon as the light dispersion of the cell suspension measured at 600 nm (O.D._{600 nm}) reached 0.4, it was infected with M13-KO7 helper phage (NEB) allowing at least 10 phages per E. coli cell on average. After shaking it for 30 minutes at 37° C. the cells were added to 30 ml 2YT/ampicillin (100 μg/ml), kanamycin (25 μg/ml) medium. The cells were shaken for 16 more hours at 37° C. Then the cells were isolated from the culture by centrifugation (10,000 rpm, 10 minutes, 4° C.), and from the supernatant containing the phages. The phages were precipitated in a clean centrifuge tube by adding 1/5 volume PEG/NaCl solution (20% PEG 8000, 2.5 M NaCl). After thoroughly mixing in the precipitation agent, the sample was left alone for 20 minutes at room temperature. Then the phage particles were settled by centrifuging (12,000 rpm, 10 minutes, 4° C.). After pouring off the supernatant carefully and putting back the tube in the same position, the liquid stuck to the wall of the tube was collected by centrifuging it for a while (1,000 rpm, 1 minute, 4° C.) and then it was removed with a pipette. The phages were suspended in 800 μl PBS, and the remaining cell fragments were removed from the sample by centrifuging it in a microcentrifuge (12,000 rpm, 10 minutes) and transferring the supernatant into a clean microcentrifuge tube. The supernatant obtained in this way contained pure phages.

D.2.1.3.1.3. Isolation of Single-Stranded DNA from Phages

From the nearly 800 μl phage, single-stranded DNA (ssDNA) was isolated with the help of a QIAprep® Spin Miniprep Kit (#27106) kit supplemented with in house prepared 2.8M citric acid and 1M sodium-perchlorate, 30% (v/v) isopropanol solutions following the manufacturer's instructions. This supplemented kit substituted for the original QIAgen Spin M13 kit (cat. no. 27704) dedicated for the use of M13 DNA isolation, but discontinued by the same manufacturer. The amount of the pure ssDNA was determined on the basis of UV light absorption at 260 nm.

### D.2.1.3.1.4. Kunkel Mutagenesis

Stop mutations were introduced with the

EVO2-stage-3-stop-1 (SEQ ID NO: 62):
CGGGCAAACCGGACTTCTGCTTCCTGGAAGAAGACCCGGGTTAATGCTA
AGCGTAAAAACTTCGTTACTTCTACAACAACCAGAC
and
the EVO2-stage-2-stop-2 (SEQ ID NO: 63):
CAAACAGTGCGAACGTTTCTAATACGGTGGTTGCTTCGGTAACATGAAC
AACTTCGTAACCCTGGAAGAATGCAAAAACATCTGCG

oligonucleotides.

### D.2.1.3.1.4.1. Oligo Phosphorylation

Both oligonucleotides were phosphorylated in separate reactions as follows.
2 μl mutagenesis oligo (330 ng/μl)
2 μl 10×TM buffer (0.5 M Tris-HCl pH 7.5, 0.1 M MgCl₂)
2 μl 10 mM ATP
1 μl 100 mM DTT
12 μl distilled water
1 μl polynucleotide kinase (NEB, 10 U/μl)

The reaction was incubated for 30 minutes at 37° C.

### D.2.1.3.1.4.2. Oligo-Template Annealing

1 μg ssDNA
2 μl from the kinase oligo reaction of both mixtures
2.5 μl 10×TM buffer
Distilled water up to a final volume of 25 μl
Incubation: 90° C. 1 minutes, 50° C. 3 minutes, then after centrifuging it for a while it was put on ice.

### D.2.1.3.1.4.3. Polymerisation and Ligation

To the DNA solution from D.2.1.3.1.4.2. first the following reagents were added:
1 μl 10 mM ATP
1.5 μl 100 mM DTT
0.6 μl T4 ligase (NEB, 400 U/μl)

The reaction was incubated for 30 minutes at room temperature in order to facilitate ligation of the two oligonucleotides. The following reagents were added to achieve second DNA strand synthesis:
1 μl 25 mM dNTP
0.6 μl T7 polymerase (NEB, 10 U/μl)

The reaction mixture was incubated for 2 hours at 37° C. The whole mixture was run on 1% agarose gel and the product of the desired size was cut out from the gel. From this piece of gel, with the help of a QIAgen Gel Extraction kit (cat. no. 28706), the Kunkel product was isolated in 30 μl elution buffer (EB). With the Kunkel product XL1 Blue cells were transformed as described in D.2.1.3.1.1., using 1 μl DNA. From individual colonies, cell cultures were grown in LB/ampicillin (100 μg/ml) medium. From the cells, the phagemid was isolated with a QIAprep® Spin Miniprep Kit (#27106), following the manufacturer's instructions. Identity and quality of the DNA construct was tested via sequencing with Big Dye Terminator v3.1 cycle Sequencing Kit (Applied Biosystems; cat #4336917) system used for the sequencing PCR reaction. The product of the sequencing reaction was run by BIOMI Kft. (Gödöllö, Hungary). The name of the vector created in this way: pEVO214a-STOP phagemid, the functionally relevant part of its sequence corresponds to SEQ ID NO: 64).

### D.2.1.3.2. Library Kunkel Mutagenesis

The library mutagenesis was performed in a similar way as described above in section D.2.1.3.1.4., but using ten times the amounts of reagents determined therein. The library oligo is analogous with the stop mutation oligo, but in this case, there are degenerate NNK and/or AVA triplets (using the IUPAC coding relating to degenerate oligonucleotides) in the place of the TAA stop codons. The sequences of the library mutagenesis oligonucleotides as the following:
Library mutagenesis oligonucleotide EVO2-stage-3-lib-1, (SEQ ID NO: 65)

CGGGCAAACCGGACTTCTGCTTCCTGGAAGAAGACCCGGGTNNKTGCAV
AGCGNNKAAACTTCGTTACTTCTACAACAACCAGAC

and
Library mutagenesis oligonucleotide EVO2-stage-3-lib-2 (SEQ ID NO: 66)

CAAACAGTGCGAACGTTTCNNKTACGGTGGTTGCTTCGGTAACATGAAC
AACTTCGTAACCCTGG

were used and oligo phosphorylation was performed as described above in D.2.1.3.1.4.1. To create the library, ten times the amount of the oligo was used in oligo-template annealing, so all the oligo created during the kinase reaction was used. The template for the mutagenesis was the uracil-containing ssDNA carrying the stop codons, which was created from the pEVO214a-STOP phagemid obtained as a result of the procedure described above in detail, in CJ236 cells, by M13K07 helper phage infection. For creating the library ten times the amount of the template was used: 20 μg and the volume of the annealing reaction was also increased by ten times to 250 μl, while keeping the same final concentration of the components. The incubation periods were extended: 90° C. for 2 minutes, 50° C. for 5 minutes. As above, a separate 30 minute room temperature ligation reaction was applied to ligate the two mutagenesis oligonucleotides, then, after adding dNTP and T7 polymerase, the mixture was incubated for 3 hours at 37° C.

The product was purified with Qiagen Gel Extraction kit. It was not isolated from gel, only purified using two columns. Elution took place in 2×30 μl USP distilled water.

### D.2.1.4. Electroporation, Multiplication of the Phage Library

The DNA library was introduced to the supercompetent cells via electroporation. Our aim was to introduce the phagemid to as many cells as possible, so that our library contains 10⁸-10⁹ individual transformed cells.

The DNA library, which was dissolved in USP distilled water so it was salt-free, was added to 2×350 μl supercompetent cells. 30 μl of library DNA was electroporated into 350 μl of supercompetent cells and the process was repeated with the other half of the DNA library. The operation was performed in an electroporation cuvette with a gap size of 0.2 cm, according to the following protocol: 2.5 kV, 200 Ohm, 25 ρF.

After electroporation, the cells were carefully transferred into 2×25 ml of SOC medium, incubated for 30 minutes by shaking at 200 rpm, at 37° C., then a 10 μl sample was taken, a tenfold, 8-member serial dilution was made from it and 10 μl from each dilution was dripped onto [LB], [LB; 100 μg/ml ampicillin] and [LB; 10 μg/ml tetracycline] plates, and it was grown overnight at 37° C. After taking the above sample, the rest of the 2×25 ml culture was infected with 2×250 μl M13KO7 helper phage (1×10¹³ PFU/ml), shaken at 37° C. for 30 minutes at 220 rpm, and then the whole product was inoculated into 2×500 ml [2YT; 100 μg/ml ampicillin; 25 μg/ml kanamycin] medium. The culture was grown in two 2-litre baffled Erlenmeyer flasks at 37° C., at 220 rpm, for 18 hours.

On the basis of titration our library contained 2.5×10⁹ variants.

### Example D.2.2.: Selection of the Library on the Human MASP-2 Enzyme and Independently, in Parallel, on the Rat MASP-2 Enzyme

### D.2.2.1. The Target Enzymes

The MASP-2 target enzymes consist of a serine-protease (SP) domain and two complement control protein domains (CCP1, CCP2) (Gal 2007). These are recombinant fragment products, which carry the catalytic activity of the entire molecule (“catalytic fragment”). The proteins were produced in the form of inclusion bodies, from which the conformation with biological activity was obtained by renaturation. Purification was performed by anion and cation exchange separation. The activity of the proteins was tested in a solution and also in a form linked to the ELISA plate. (For the amino acid sequence and the precise details of production of the human MASP-2 target see Ambrus 2003). The rat MASP-2 target was produced similarly to the human target. The catalytic fragment of rat MASP-2 starts with Gln298 and ends with Phe685 according to UniProt numbering (entry Q9JJS8). Cloning was carried out as in the case of human MASP-2 described in Ambrus 2003. As a result of cloning, the recombinant protein was produced with an extra Met-Thr dipeptide segment at the N terminus. The rat recombinant MASP-2 protein was expressed, refolded and purified following the procedure used earlier at the human protein fragment.

The data of the targets used during selection are the following:
human MASP-2cf CCP1-CCP2-SP: Mw=44017 Da, c_{stock}=3.0 g/l
rat MASP-2cf CCP1-CCP2-SP: Mw=42309 Da, c_{stock}=3.6 g/l

### D.2.2.2. Steps of Selection

### D.2.2.2.1. Isolating the Phages

At the end of the operation described in chapter D.2.1.3, phages were produced in 2×500 ml of culture for 18 hours. In the first step of the selection they were isolated to enable the use of the library immediately for selection.

The cell culture was centrifuged at 8,000 rpm for 10 minutes, at 4° C. The supernatant, which contained bacteriophages, was poured into clean centrifuge tubes, and a precipitating agent ⅕^{th} of its volume was added to it [2.5 M NaCl; 20% PEG-8000]. Precipitation took place at room temperature, for 20 minutes. Then it was centrifuged again at 10,000 rpm for 10 minutes, at 4° C. The supernatant was discarded, it was centrifuged again for a short time, and the remaining liquid was pipetted off. The white phage precipitate was solubilised in 25 ml [PBS; 5 mg/ml BSA; 0.05% Tween-20] buffer. In order to remove residual cell debris, it was centrifuged again at 12,000 rpm for 10 minutes and the supernatant was transferred into clean tubes.

### D.2.2.2.2. The First Selection Cycle

a) Immobilisation: The target molecules were immobilised in separate wells on a 96-well Nunc Maxisorp ELISA plate (cat #442404). During immobilisation, the concentration of human MASP-2cf and rat MASP-2cf was 20 μg/ml. Both proteins were diluted in the immobilisation buffer [200 mM Na₂CO₃; pH 9.4], and 100 μl was put in the wells. Both human and rat MASP-2cf were incubated overnight at 4° C. In the first selection cycle twelve wells per target protein were used. Every second row was left empty. As negative control only immobilising buffer was put in one row. This row was then treated the same way as the ones covered with target protein.
b) Blocking: The immobilising solution was removed, and 200 μl/well of blocking buffer [PBS; 5 mg/ml BSA] was put onto the plate. It was incubated at room temperature while mixing at 150 rev/min for at least 1 hour.
c) Washing: The ELISA-plate was washed 4 times using 1 l of wash buffer [PBS; 0.05% Tween-20].
d) Selection: The phages of the library isolated as described above were pipetted onto the plate, 100 μl in each well. It was incubated at room temperature while mixing at 110 rev/min, for 3 hours.
e) E. coli XL1 Blue culture: During the term of the selection, XLI Blue cells were inoculated from a plate freshly picked in advance using an inoculating loop, into 2×30 ml of medium [2YT; 10 μg/ml tetracycline]. These cells were to be infected with phages eluted from the target proteins. At the time of infection, the cells must be in the phase of exponential growth. A culture with O.D._{600 nm}˜0.3-0.5 was needed, which was obtained by growing it at 37° C., at 220 rpm, for 2-3 hours.
f) Washing: The ELISA-plate was washed 12 times using 3 litres of wash buffer.
g) Elution: Elution was performed using 100 mM HCl solution, 100 μl/well. The acid was applied, shaken for 5 minutes, and then it was drawn from each well one by one. The phages eluted from the individual target proteins were collected in a tube, in which 12×15 μl 1 M Tris-base buffer had been put in advance to quickly neutralise the acid solution containing the phages. The tubes were immediately mixed and placed on ice.
h) Infection: 4.5 ml of XL1 Blue culture in the phase of exponential growth was put in test tubes, and it was infected with 500 μl of phage solution eluted from the target protein. A total number of three infections were performed, with phages eluted from human MASP-2, from rat MASP-2 and from the negative control substance. The cultures were incubated at 37° C., at 220 rpm, for 30 minutes.
i) Titration: A 20 μl sample was taken from each infected culture, it was diluted to 10 times its volume with 2YT medium, and a sequence was prepared with further 10× dilutions. From each point 10 μl was dripped onto a plate [LB agar; 100 μg/ml ampicillin] and grown overnight at 37° C.
j) Infection with helper phage: Directly after sampling, 50 μl M13KO7 (1×10¹³ PFU/ml) helper phage was added to each culture in the test tubes, and the cultures were incubated for an additional 30 minutes.
k) All infected cultures were transferred into 200 ml medium [2YT; 100 μg/ml ampicillin; 30 μg/ml kanamycin] and incubated at 37° C. while mixing at 220 rpm, for 18 hours. The control substance was not treated any further as it was only needed for titration.
l) Enrichment: On the following morning the results of the titration were evaluated. The number of phages eluted from the human MASP-2, i.e., the number of phage-infected and thereby ampicillin resistant colonies, was about 100-fold higher than that of phages eluted from the BSA background, while in the case of rat MASP-2 this ratio was about 10. This ratio is referred to as the enrichment value.

### D.2.2.2.3. The Second Selection Cycle

In this cycle, the same steps were repeated as in the case of the first selection cycle but only eight wells/target were used. In this step, each target protein had its own control substance (eight wells), and the phages eluted and multiplied in the previous cycle were placed both on the target and the control protein.

The phages produced for 18 hours were isolated as described above, but at the end they were solubilised in 10 ml of sterile PBT buffer. After the second selection cycle 2.7 ml of fresh exponentially growing XL1 Blue cells was infected with 300 μl of eluted phage. Titration was performed in all four cases (2 target proteins+2 control substances), and then the cultures also infected with helper phage were transferred into 30 ml [2YT; 100 μg/ml ampicillin; 30 μg/ml kanamycin] medium.

After the second selection cycle we determined the number of clones eluted from the specific target coated, BSA blocked wells and divided this number with the number of clones eluted from the target-free, BSA-blocked wells. We obtained an enrichment value of 310-470 for human MASP-2, and a value of 120-130 for rat MASP-2.

### D.2.2.3. Testing Individual Clones Using Phage ELISA Assay

In the ELISA test, we were looking for phage clones that are able to bind strongly to their own target protein, while they display significantly lower signals on the BSA coated control surface.
a) Infection: In the case of both the human MASP-2 and the rat MASP-2, eluted phages from selection cycle 1 and selection cycle 2 were used to produce clonally homogeneous phage solutions. For this, 10 μl of eluted phage from selection cycle 1 and 10 μl of eluted phage from selection cycle 2 were added to separate 250 pls of XL1 Blue cultures being in exponential phase. The eluted phages were diluted previously to contain 200-400 phages/10 μl in order to ensure the high excess of cells during infection. The infected cells were incubated for 30 minutes at 37° C. while mixing the suspension at 220 rpm. Then the cells were spread onto [LB; 100 μg/ml ampicillin] plates, and they were grown overnight at 37° C.
b) Inoculation: individual colonies were inoculated into so-called “single loose” tubes (National Scientific Supply—TN0933-C01) containing 500 μl of medium [2YT; 100 μg/ml ampicillin; 10¹⁰ phage/ml M13KO7 helper phage]. These tubes were arranged similarly to a 96-well ELISA-plate arrangement, they can rotate individually, so in a plate incubator, at 37° C., while mixing at 300 rev/min, the samples in the tubes are intensely aerated and are suitable for producing small-volume cultures.
c) Immobilisation: both the human MASP-2 and the rat MASP-2 proteins were immobilised at a concentration of 10 μg/ml in 50 μl/well volume, as described above in connection with selection, on Nunc ELISA Maxisorp plates. Each clone was tested on its own target protein, on the other target protein and on BSA coated control surface.
d) After 18 hours, the “single loose” tubes were centrifuged in a rotor suitable for accommodating ELISA plates at 2,500 rpm, for 10 minutes, at 4° C., the supernatant was pipetted into clean tubes. After testing aliquots taken from each tube on phage-ELISA the remaining supernatant, in the interest of killing off the contaminating E. coli bacteria was heated for 2 hours at 65° C. After this, the samples were stored at −20° C., until used for sequencing.
e) Blocking: The liquid was removed from the immobilised samples, and 200 μl/well of [PBS; 5 mg/ml BSA] blocking buffer was placed in each well. Incubation took place at room temperature, for at least 1 hour, while mixing at 150 rev/min.
f) Washing: The plate was washed 4 times using 1 litre of wash buffer.
g) Phage application: 50-50 μl of the phages produced and isolated as described above were placed in the wells. From the same clone samples were pipetted into a total of 3 wells, i.e., self-target, other target and BSA control. Incubation was performed at room temperature, for 1 hour while mixing at 110 rev/min.
h) Washing: The plate was washed 6 times using 1.5 litres of wash buffer.
i) Anti-M13 antibody: 50 μl of monoclonal anti-M13 HRP conjugated antibody (Amersham, cat #27-9421-01) 5,000 times diluted in buffer [PBS; 5 mg/ml BSA; 0.05% Tween-20] was placed in the wells, and then it was incubated for 30 minutes at room temperature while mixing at 110 rev/min.
j) Washing: The plate was washed 6 times with 1.5 litres of wash buffer, and then twice with PBS.
k) Signal development: 50 μl of 1-Step Ultra TMB-ELISA substrate (Pierce, cat #34028) was placed in each well, shaken for a while, and then the reaction was stopped by adding 50 μl of 1M HCl in each well.
l) Reading: absorbance was measured at 450 nm, using BioTek Epoch (Agilent) plate reading photometer.

We took a sample from those phage supernatants that produced low intensity signal on the BSA background while displaying at least two times higher intensity signal on their own target protein, and prepared these samples for DNA sequencing. We used 2 μl of supernatant and used the Big Dye Terminator v3.1 cycle Sequencing Kit (Applied Biosystems; cat #4336917) system for the sequencing PCR reaction. The samples were analysed by BIOMI Kft. (Gödöllö, Hungary).

### Example D.2.3: Results

In this example, we describe the results of the tests described in Examples D.2.1. and D.2.2., that is, the sequences obtained.

From the phages eluted from human MASP-2 we tested 224 clones, 112 from selection cycle 1 and 112 from selection cycle 2 using ELISA, and finally we found 192 individual sequences. In the case of rat MASP-2 we tested 128 clones, 108 was ELISA-positive corresponding to 98 individual sequences.

When interpreting the results, we had to take into consideration that the NNK codon pattern used when constructing the DNA library does not ensure the same initial frequency for the twenty individual amino acids. In the NNK codon pattern an amino acid may have one, two or three codons. Therefore, we performed codon normalisation by dividing all amino acid frequencies by the number of codons the given amino acid is represented by in the NNK set.

After data normalisation, we made sequence logo diagrams about the sequences with the help of WebLogo (Crooks 2004) accessible on the internet (http://weblogo.berkeley.edu/logo.cgi).

The sequence logo is the graphic display of the information content and amino acid distribution per position in a set of multiple aligned sequences, using the single-letter abbreviations of the amino acids. In each position, the column height of the logo indicates how even the occurrence of the elements (twenty different types of amino acids in our case for x13, x17 and x34 and three different types for x15). The less even this occurrence is, the higher the column. In the case of completely even distribution (all allowed amino acids occur in equal proportion) the height is zero. The maximum value belongs to the case, where only one type of element (amino acid) occurs. Within the column the individual amino acids are arranged on the basis of the frequency of occurrence, the most frequent one is at the top. The height of the letter indicating the amino acid is in proportion with its relative frequency of occurrence in the given position (for example, in the case of 50% frequency of occurrence, it is half the height of the column). In the case of colour diagrams, generally amino acids with similar chemical characteristics are shown in the same or in a similar colour, for which we used different shades of grey in the figure belonging to the present patent description.

On the horizontal axis of the usual sequence logo diagrams the number of the individual positions of the randomised region can be seen, site P1 corresponds to position x15. On the vertical axis, the information content of the positions is determined in bits.

In a transformed version of the sequence logo diagram, all columns heights are set equal allowing for a better visualization of rarely selected amino acid types especially at positions where the level of conservation is low, and therefore the height of the column and the stacked letters therein are small rendering the letters unreadable.

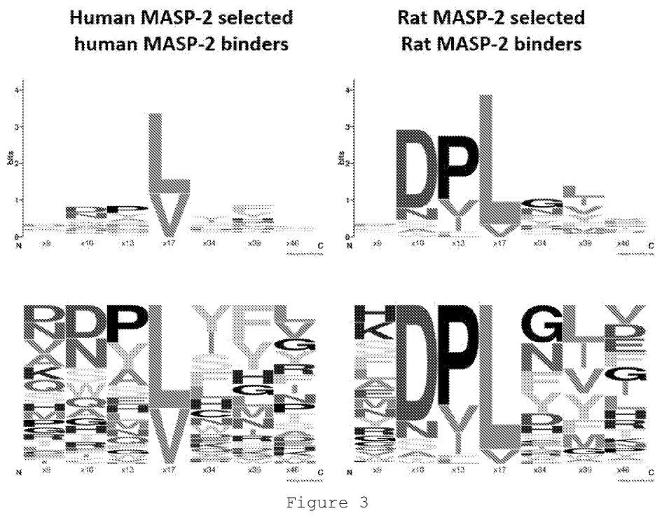

The logos for all three phage display evolution stages are shown in FIGS. 3, 4 and 5.

Codon normalised frequencies of each amino acid at each randomized positions are also listed for all three stages in Tables 3 to 9.

TABLE 3
Stage 1 phage display evolution: codon normalised amino
acid frequencies representing the set of sixty-four human
MASP-2 enzyme selected human MASP-2 binding clones

| Position | x9 | x10 | x13 (P3) | x17 (P2′) | x34 | x39 | x46 |
|---|---|---|---|---|---|---|---|
| Amino acid | N | D | P | L | Y | F | L |
| (frequency) | (10%) | (21%) | (24%) | (65%) | (17%) | (22%) | (11%) |
| Amino acid | D | N | Y | V | I | Y | V |
| (frequency) | (10%) | (19%) | (16%) | (35%) | (14%) | (18%) | (10%) |
| Amino acid | V | W | A | | S | H | G |
| (frequency) | (9%) | (9%) | (11%) | | (11%) | (9%) | (9%) |
| Amino acid | A | S | S | | F | G | Y |
| (frequency) | (8%) | (9%) | (7%) | | (10%) | (7%) | (9%) |
| Amino acid | Q | Q | L | | L | L | R |
| (frequency) | (8%) | (7%) | (5%) | | (8%) | (7%) | (7%) |
| Amino acid | K | A | M | | H | M | I |
| (frequency) | (8%) | (6%) | (5%) | | (7%) | (7%) | (6%) |
| Amino acid | S | G | N | | W | N | F |
| (frequency) | (7%) | (5%) | (5%) | | (5%) | (7%) | (6%) |
| Amino acid | M | T | Q | | C | I | T |
| (frequency) | (5%) | (5%) | (5%) | | (5%) | (4%) | (6%) |
| Amino acid | P | H | E | | N | S | P |
| (frequency) | (5%) | (5%) | (5%) | | (5%) | (4%) | (6%) |
| Amino acid | H | R | H | | Q | R | N |
| (frequency) | (5%) | (4%) | (5%) | | (3%) | (4%) | (6%) |
| Amino acid | G | I | G | | D | W | S |
| (frequency) | (4%) | (2%) | (3%) | | (3%) | (2%) | (5%) |
| Amino acid | T | F | F | | E | Q | A |
| (frequency) | (4%) | (2%) | (3%) | | (3%) | (2%) | (4%) |
| Amino acid | R | Y | W | | K | D | M |
| (frequency) | (4%) | (2%) | (3%) | | (3%) | (2%) | (3%) |
| Amino acid | L | E | V | | V | K | W |
| (frequency) | (3%) | (2%) | (1%) | | (2%) | (2%) | (3%) |
| Amino acid | I | V | R | | R | V | D |
| (frequency) | (3%) | (1%) | (1%) | | (2%) | (1%) | (3%) |
| Amino acid | F | L | | | G | | E |
| (frequency) | (3%) | (1%) | | | (1%) | | (3%) |
| Amino acid | C | | | | A | | H |
| (frequency) | (3%) | | | | (1%) | | (3%) |
| Amino acid | E | | | | T | | K |
| (frequency) | (3%) | | | | (1%) | | (3%) |
| Amino acid | | | | | P | | |
| (frequency) | | | | | (1%) | | |

TABLE 4
Stage 1 phage display evolution: codon normalised amino acid frequencies representing
the set of fifty-five rat MASP-2 enzyme selected rat MASP-2 binding clones

| Position | x9 | x10 | x13 (P3) | x17 (P2′) | x34 | x39 | x46 |
|---|---|---|---|---|---|---|---|
| Amino acid | H | D | P | L | G | L | V |
| (frequency) | (11%) | (73%) | (63%) | (91%) | (24%) | (24%) | (12%) |
| Amino acid | K | N | V | V | N | V | G |
| (frequency) | (11%) | (11%) | (17%) | (9%) | (18%) | (16%) | (9%) |
| Amino acid | S | S | I | | F | I | L |
| (frequency) | (10%) | (8%) | (14%) | | (13%) | (16%) | (9%) |
| Amino acid | F | A | L | | Y | Y | I |
| (frequency) | (9%) | (3%) | (3%) | | (13%) | (16%) | (9%) |
| Amino acid | L | W | F | | D | M | F |
| (frequency) | (7%) | (2%) | (3%) | | (8%) | (9%) | (9%) |
| Amino acid | A | T | | | H | F | D |
| (frequency) | (6%) | (2%) | | | (5%) | (9%) | (9%) |
| Amino acid | M | | | | I | G | E |
| (frequency) | (6%) | | | | (3%) | (5%) | (9%) |
| Amino acid | Y | | | | W | W | T |
| (frequency) | (6%) | | | | (3%) | (3%) | (6%) |
| Amino acid | N | | | | S | A | H |
| (frequency) | (6%) | | | | (3%) | (2%) | (6%) |
| Amino acid | E | | | | C | S | R |
| (frequency) | (6%) | | | | (3%) | (1%) | (6%) |
| Amino acid | R | | | | Q | | Y |
| (frequency) | (5%) | | | | (3%) | | (3%) |
| Amino acid | G | | | | E | | P |
| (frequency) | (4%) | | | | (3%) | | (3%) |
| Amino acid | V | | | | R | | N |
| (frequency) | (3%) | | | | (2%) | | (3%) |
| Amino acid | W | | | | A | | K |
| (frequency) | (3%) | | | | (1%) | | (3%) |
| Amino acid | C | | | | L | | A |
| (frequency) | (3%) | | | | (1%) | | (2%) |
| Amino acid | Q | | | | P | | S |
| (frequency) | (3%) | | | | (1%) | | (2%) |
| Amino acid | T | | | | | | |
| (frequency) | (1%) | | | | | | |
| Amino acid | P | | | | | | |
| (frequency) | (1%) | | | | | | |

TABLE 5
Stage 2 phage display evolution: codon normalised amino
acid frequencies representing the set of fifty-four human
MASP-2 enzyme selected human MASP-2 binding clones

| Position | X13 (P3) | x15 (P1) | x16 (P1′) | x17 (P2′) | x18 (P3′) | x19 (P4′) |
|---|---|---|---|---|---|---|
| Amino acid | W | R | A | A | K | A |
| (frequency) | (19%) | (66%) | (24%) | (25%) | (14%) | (12%) |
| Amino acid | L | K | G | H | A | L |
| (frequency) | (14%) | (34%) | (16%) | (15%) | (10%) | (10%) |
| Amino acid | M | | S | Y | I | M |
| (frequency) | (14%) | | (14%) | (12%) | (8%) | (9%) |
| Amino acid | F | | N | L | M | N |
| (frequency) | (11%) | | (14%) | (10%) | (8%) | (9%) |
| Amino acid | Y | | I | I | T | Q |
| (frequency) | (11%) | | (10%) | (9%) | (8%) | (9%) |
| Amino acid | P | | M | G | R | D |
| (frequency) | (5%) | | (7%) | (7%) | (6%) | (9%) |
| Amino acid | H | | L | C | F | V |
| (frequency) | (5%) | | (6%) | (6%) | (5%) | (6%) |
| Amino acid | K | | V | V | Y | E |
| (frequency) | (5%) | | (3%) | (3%) | (5%) | (6%) |
| Amino acid | A | | K | M | N | K |
| (frequency) | (4%) | | (3%) | (3%) | (5%) | (6%) |
| Amino acid | V | | R | F | Q | G |
| (frequency) | (4%) | | (2%) | (3%) | (5%) | (5%) |
| Amino acid | S | | | W | D | T |
| (frequency) | (3%) | | | (3%) | (5%) | (5%) |
| Amino acid | E | | | N | E | R |
| (frequency) | (3%) | | | (3%) | (5%) | (5%) |
| Amino acid | T | | | S | V | Y |
| (frequency) | (1%) | | | (1%) | (4%) | (3%) |
| Amino acid | | | | R | L | W |
| (frequency) | | | | (1%) | (4%) | (3%) |
| Amino acid | | | | | W | P |
| (frequency) | | | | | (3%) | (3%) |
| Amino acid | | | | | S | S |
| (frequency) | | | | | (2%) | (1%) |
| Amino acid | | | | | P | |
| (frequency) | | | | | (1%) | |

TABLE 6
Stage 2 phage display evolution: codon normalised amino acid frequencies representing
the set of sixty-eight rat MASP-2 enzyme selected rat MASP-2 binding clones

| Position | X13 (P3) | x15 (P1) | x16 (P1′) | x17 (P2′) | x18 (P3′) | x19 (P4′) |
|---|---|---|---|---|---|---|
| Amino acid | V | R | A | A | A | V |
| (frequency) | (40%) | (100%) | (59%) | (49%) | (28%) | (21%) |
| Amino acid | P | | V | L | W | Q |
| (frequency) | (32%) | | (17%) | (15%) | (10%) | (10%) |
| Amino acid | I | | G | Y | V | K |
| (frequency) | (16%) | | (9%) | (11%) | (7%) | (10%) |
| Amino acid | L | | S | H | T | C |
| (frequency) | (4%) | | (7%) | (11%) | (7%) | (8%) |
| Amino acid | Y | | M | I | Q | R |
| (frequency) | (3%) | | (3%) | (8%) | (7%) | (8%) |
| Amino acid | Q | | L | F | D | L |
| (frequency) | (3%) | | (2%) | (3%) | (7%) | (7%) |
| Amino acid | A | | T | C | S | I |
| (frequency) | (1%) | | (2%) | (3%) | (6%) | (5%) |
| Amino acid | | | R | V | I | H |
| (frequency) | | | (1%) | (1%) | (5%) | (5%) |
| Amino acid | | | | | M | G |
| (frequency) | | | | | (5%) | (4%) |
| Amino acid | | | | | F | A |
| (frequency) | | | | | (5%) | (4%) |
| Amino acid | | | | | G | S |
| (frequency) | | | | | (4%) | (4%) |
| Amino acid | | | | | L | Y |
| (frequency) | | | | | (3%) | (3%) |
| Amino acid | | | | | E | W |
| (frequency) | | | | | (2%) | (3%) |
| Amino acid | | | | | H | T |
| (frequency) | | | | | (2%) | (3%) |
| Amino acid | | | | | K | N |
| (frequency) | | | | | (2%) | (3%) |
| Amino acid | | | | | | D |
| (frequency) | | | | | | (3%) |
| Amino acid | | | | | | P |
| (frequency) | | | | | | (1%) |

TABLE 7
Stage 3 phage display evolution: codon normalised amino acid
frequencies representing the set of one-hundred-ninety-two
human MASP-2 enzyme selected human MASP-2 binding clones.

| | x3 | x15 | x17 | |
|---|---|---|---|---|
| Position | (P3) | (P1) | (P2′) | x34 |
| Amino acid | F | R | I | I |
| (frequency) | (36%) | (70%) | (16%) | (18%) |
| Amino acid | Y | K | L | Y |
| (frequency) | (19%) | (30%) | (12%) | (15%) |
| Amino acid | M | T | F | F |
| (frequency) | (7%) | (1%) | (10%) | (12%) |
| Amino acid | H | | V | N |
| (frequency) | (7%) | | (8%) | (6%) |
| Amino acid | L | | M | K |
| (frequency) | (6%) | | (8%) | (6%) |
| Amino acid | A | | A | V |
| (frequency) | (4%) | | (7%) | (5%) |
| Amino acid | V | | Y | M |
| (frequency) | (4%) | | (7%) | (5%) |
| Amino acid | P | | H | D |
| (frequency) | (4%) | | (6%) | (5%) |
| Amino acid | W | | G | G |
| (frequency) | (3%) | | (4%) | (4%) |
| Amino acid | Q | | W | L |
| (frequency) | (2%) | | (4%) | (4%) |
| Amino acid | G | | S | S |
| (frequency) | (1%) | | (4%) | (4%) |
| Amino acid | I | | C | H |
| (frequency) | (1%) | | (4%) | (4%) |
| Amino acid | S | | T | C |
| (frequency) | (1%) | | (2%) | (3%) |
| Amino acid | T | | N | T |
| (frequency) | (1%) | | (2%) | (2%) |
| Amino acid | N | | R | P |
| (frequency) | (1%) | | (2%) | (2%) |
| Amino acid | D | | Q | E |
| (frequency) | (1%) | | (1%) | (2%) |
| Amino acid | K | | D | A |
| (frequency) | (1%) | | (1%) | (1%) |
| Amino acid | | | E | W |
| (frequency) | | | (1%) | (1%) |
| Amino acid | | | K | Q |
| (frequency) | | | (1%) | (1%) |
| Amino acid | | | | R |
| (frequency) | | | | (1%) |

TABLE 8
Stage 3 phage display evolution: codon normalised amino
acid frequencies representing the set of ninety-eight
rat MASP-2 enzyme selected rat MASP-2 binding clones.

| | x3 | x15 | x17 | |
|---|---|---|---|---|
| Position | (P3) | (P1) | (P2′) | x34 |
| Amino acid | P | R | F | Y |
| (frequency) | (27%) | (96%) | (20%) | (24%) |
| Amino acid | V | K | Y | F |
| (frequency) | (26%) | (4%) | (19%) | (17%) |
| Amino acid | F | | L | I |
| (frequency) | (12%) | | (18%) | (15%) |
| Amino acid | I | | A | G |
| (frequency) | (10%) | | (14%) | (9%) |
| Amino acid | M | | I | V |
| (frequency) | (7%) | | (11%) | (7%) |
| Amino acid | L | | H | D |
| (frequency) | (6%) | | (8%) | (4%) |
| Amino acid | Y | | M | A |
| (frequency) | (3%) | | (5%) | (3%) |
| Amino acid | N | | C | M |
| (frequency) | (2%) | | (3%) | (3%) |
| Amino acid | H | | V | T |
| (frequency) | (2%) | | (2%) | (3%) |
| Amino acid | G | | W | C |
| (frequency) | (1%) | | (2%) | (3%) |
| Amino acid | T | | S | N |
| (frequency) | (1%) | | (1%) | (3%) |
| Amino acid | Q | | | H |
| (frequency) | (1%) | | | (3%) |
| Amino acid | R | | | L |
| (frequency) | (1%) | | | (2%) |
| Amino acid | | | | S |
| (frequency) | | | | (2%) |
| Amino acid | | | | W |
| (frequency) | | | | (1%) |
| Amino acid | | | | P |
| (frequency) | | | | (1%) |
| Amino acid | | | | E |
| (frequency) | | | | (1%) |

TABLE 9
Stage 3 phage display evolution: codon normalised amino
acid frequencies representing the cumulative set of
two-hundred-ninety human MASP-2 binding clones that
were selected either on rat or human MASP-2.

| | x3 | x15 | x17 | |
|---|---|---|---|---|
| Position | (P3) | (P1) | (P2′) | x34 |
| Amino acid | F | R | L | Y |
| (frequency) | (29%) | (79%) | (14%) | (18%) |
| Amino acid | Y | K | I | I |
| (frequency) | (15%) | (21%) | (14%) | (17%) |
| Amino acid | P | T | F | F |
| (frequency) | (11%) | (0%) | (13%) | (14%) |
| Amino acid | V | | Y | G |
| (frequency) | (10%) | | (11%) | (6%) |
| Amino acid | M | | A | V |
| (frequency) | (7%) | | (10%) | (6%) |
| Amino acid | L | | M | N |
| (frequency) | (6%) | | (7%) | (5%) |
| Amino acid | H | | V | D |
| (frequency) | (6%) | | (6%) | (5%) |
| Amino acid | I | | H | L |
| (frequency) | (4%) | | (6%) | (4%) |
| Amino acid | A | | C | M |
| (frequency) | (3%) | | (4%) | (4%) |
| Amino acid | W | | G | H |
| (frequency) | (2%) | | (3%) | (4%) |
| Amino acid | Q | | W | K |
| (frequency) | (2%) | | (3%) | (4%) |
| Amino acid | G | | S | S |
| (frequency) | (1%) | | (3%) | (3%) |
| Amino acid | S | | N | T |
| (frequency) | (1%) | | (2%) | (3%) |
| Amino acid | T | | T | C |
| (frequency) | (1%) | | (1%) | (3%) |
| Amino acid | N | | Q | A |
| (frequency) | (1%) | | (1%) | (2%) |
| Amino acid | K | | D | E |
| (frequency) | (1%) | | (1%) | (2%) |
| Amino acid | R | | E | W |
| (frequency) | (1%) | | (1%) | (1%) |
| Amino acid | | | K | P |
| (frequency) | | | (1%) | (1%) |
| Amino acid | | | R | Q |
| (frequency) | | | (1%) | (1%) |
| Amino acid | | | | R |
| (frequency) | | | | (1%) |

With the logos and tables we examined, which amino acids were preferred at the individual positions and how much they differed from each other depending on whether they derived from human MASP-2 or rat MASP-2 selections.

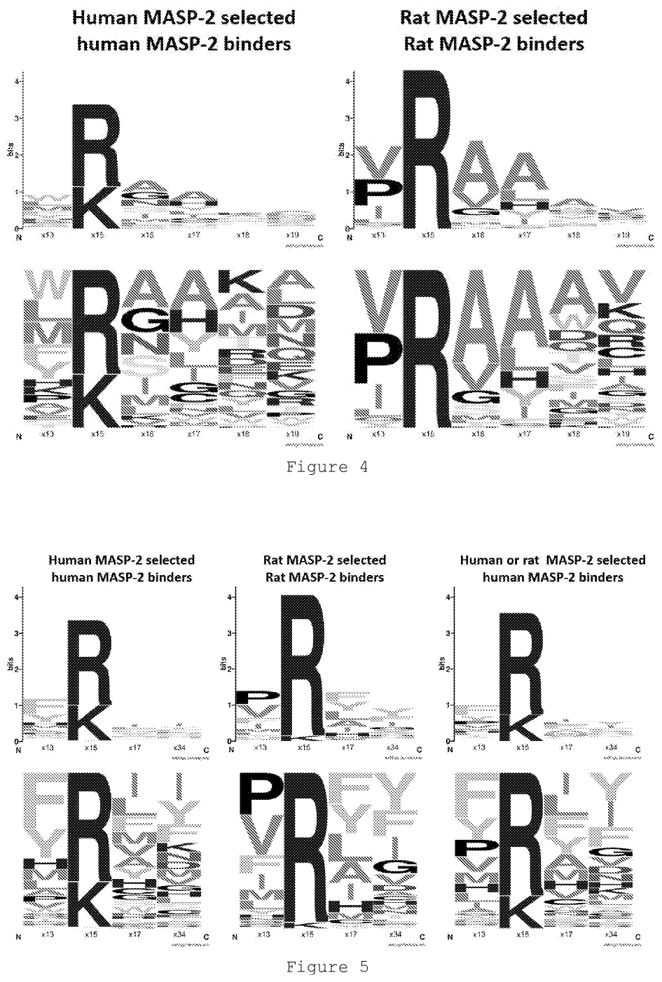

For each phage display evolution stage the results are shown in the above explained usual and transformed sequence logo diagram versions and tables. For each phage display evolution stage a pair of one usual and one transformed logo is drawn for both human MASP-2 and rat MASP-2 selection. Tables 3 and 4 and the corresponding FIG. 3, illustrate the results of the first, while Tables 5 and 6 and the corresponding FIG. 4 illustrate the results of the second evolution stage. For the third evolution stage, besides the separate analyses of human MASP-2 and rat MASP-2 selected clones, a third set corresponding to all human MASP-2 binding clones regardless whether selected on human MASP-2 or rat MASP-2 was also analysed. Accordingly, for this last evolution stage three tables are disclosed here, namely Tables 7, 8 and 9, and one extra pair of logos shown in FIG. 5.

Here, we focus on the results presented in Table 9 and the corresponding FIG. 5 logos as these are the most relevant regarding the present invention, i.e., the ones relating to the complete set of human MASP-2 binding clones regardless whether those were originally selected on the human or the rat MASP-2 enzyme.

The logo diagrams illustrate the selection taking place in the individual positions.

At x15, the P1 position out of the allowed R/K/T no T (threonine) was selected, R (arginine) was selected in 79%, while K (lysine) in 21% of the clones strongly suggesting that when being part of the Kunitz domain, an R in the P1 position is the most favoured amino acid for the S1 pocket of human MASP-2.

At x13, the P3 position, the human MASP-2 binders closely recapitulate the finding of the original invention presented in WO2018127719. Namely, the entire set of the general (Ih) sequence of WO2018127719 at position x13 (F, Y, L, P, Q, M, V, Q, A, T) is represented in the set of amino acids occurring at x13 of the human MASP-2 binders, and the most selected three amino acids, F/Y/P compose a subset of the most selected four amino acid F/Y/L/P set of the original invention presented in WO2018127719.

This suggests that simultaneous randomization of the x13/x15/x17/x34 positions did not alter the optimal x13 (P3) amino acid set of the Kunitz domain protein in terms of binding to human MASP-2 compared to the case, when x34 was fixed as a K in WO2018127719.

In contrast, at x34, when according to the present invention the x13/x15/x17/x34 positions were simultaneous randomized, and evolved, the human MASP-2 binding clone-set was enriched in variants carrying a markedly hydrophobic set of Y/I/F/G/V amino acids, and the original hydrophilic and charged K was not preferred. This suggested that physicochemical properties of the original K34 were suboptimal for human MASP-2 binding. Moreover, simultaneous evolution of the x13/x15/x17/x34 positions also characteristically altered the amino acid set at x17 optimal for human MASP-2 binding. In the invention described in WO2018127719, this set was V, A, I, L, M, D, H, S, while upon co-evolving it with x34, it became I, L, F, Y, A.

As compared to their original amino acid preference revealed in the invention described in WO2018127719, positions x13 and x15 did not show significantly altered amino acid preference in the present invention, we concluded that from these four positions, only x17 and x34 are strongly coupled functionally, namely, certain x17/x34 amino acid residue pairs synergistically boost MASP-2 binding affinity.

We analysed what is the common physicochemical property of the most preferred x17/x34 pairs and identified a combination of two properties: combined hydrophobic nature and cumulative side chain volume.

For calculating the cumulative side chain volume of the x17/x34 side chain pairs we used the side chains volumes from the work of (Harpaz 1994), where volume excess compared to glycine was considered, which, for Gly is zero as shown in Table 10:

TABLE 10
Amino acid side chain volume data
from the work of (Harpaz 1994).

| residue | side chain |
|---|---|
| code | volume (Å³) |
| G | 0.0 |
| A | 26.3 |
| V | 75.3 |
| L | 100.8 |
| I | 101.1 |
| P | 59.3 |
| M | 103.9 |
| C* | 49.4 |
| F | 129.7 |
| Y | 133.3 |
| W | 167.9 |
| S | 30.4 |
| T | 56.2 |
| H | 95.5 |
| N | 63.7 |
| D | 53.3 |
| Q | 85.6 |
| E | 77.0 |
| R | 129.0 |
| K | 106.2 |

*Volume of C corresponds to the reduced form.

Cumulative side chain volume was determined for all 290 clones capable of binding to human MASP-2 irrespective whether they were selected on human or rat MASP-2. Then, this set was distributed in groups according to cumulative side chain volumes rounded to a decimal place. The baseline cumulative side chain distribution in the starting library prior the selection was estimated as follows. The initial library had x17/x34 amino acid pairs corresponding to 32×32 codon pairs derived from the NNK codon set. (The translation of the TAG codon was considered Q as a result of the supE44 mutation of the XL-1 Blue strain.) We assigned and grouped the corresponding cumulative volumes to the above sets and normalised the 960 data to 290 clones to match the number of the human MASP-2 binding clones. Observed numbers of clones for each cumulative size range approximate the density distribution of the selected population, which is compared to the density distribution calculated for the initial library (see Table 11 and FIG. 6).

TABLE 11
Number of x17/x34 amino acid pairs in each cumulative
sidechain volume group. Bold indicated cumulative
sizes preferred by human MASP-2.

| | number of residue | number of residue |
|---|---|---|
| cumulative | pairs in the size | pairs in the size |
| volume groups | group calculated for | group observed after |
| (Å³) | the starting library | selection |
| 0 | 1.1 | 0 |
| 10 | 0 | 0 |
| 20 | 0 | 0 |
| 30 | 5.7 | 2 |
| 40 | 0 | 0 |
| 50 | 3.4 | 0 |
| 60 | 11.6 | 5 |
| 70 | 0 | 0 |
| 80 | 11.3 | 4 |
| 90 | 14.2 | 2 |
| 100 | 11.0 | 25 |
| 110 | 15.0 | 4 |
| 120 | 11.9 | 3 |
| 130 | 30.9 | 44 |
| 140 | 11.9 | 4 |
| 150 | 11.6 | 12 |
| 160 | 34.6 | 39 |
| 170 | 6.2 | 3 |
| 180 | 16.4 | 21 |
| 190 | 22.4 | 10 |
| 200 | 15.3 | 21 |
| 210 | 12.5 | 25 |
| 220 | 6.2 | 1 |
| 230 | 16.4 | 49 |
| 240 | 5.1 | 3 |
| 250 | 1.1 | 0 |
| 260 | 7.4 | 5 |
| 270 | 3.7 | 6 |
| 280 | 0 | 0 |
| 290 | 0 | 0 |
| 300 | 2.8 | 2 |
| 310 | 0 | 0 |
| 320 | 0 | 0 |
| 330 | 0 | 0 |
| 340 | 0.3 | 0 |

We found eight cumulative side chain size groups that are preferred for human MASP-2 binding: 100, 130, 160, 180, 200, 210, 230, and 270, where the numbers correspond to Å³.

The five most selected amino acid types at x17 and x34 gave twenty-five (x17/x34) amino acid pairs, which is a small 1/16 subset of the possible 400 amino acid pairs. Out of these twenty-five pairs twenty-one have cumulative side chain sizes that are overrepresented in the human MASP-2 binding clones as illustrated in FIG. 6. The other four pairs represent inherently low abundance sets in the initial library as A17/G34 represents an extremely small size while F17/F34, F17/Y34 and Y17/F34, an extremely large size category. As the number of the expected amino acid pairs in the initial library is inherently small, only disproportionally strong positive selection could generate statistically significant overrepresentation of these amino acid combinations.

For production, we used an expression system created by us. For more information on it see Example E.

### Example E: Heterologous Expression of the Inhibitors

All enzymes and reagents were obtained from Fermentas/Thermo Scientific. The reactions were performed according to the company's instructions. During the PCR reactions annealing took place at 50° C. for 30 seconds, 30 cycles were performed with an Esco Swift Mini device. Fermentas/Thermo Scientific GeneJet PCR purification kit (#K0701), Gel extraction kit (#K0691) and Plasmid miniprep kit (#K0502) were used for DNA isolation according to the manufacturer's instructions. All DNA constructs were verified by Sanger sequencing using ABI PRISM BigDye Terminator v3.1 Ready Reaction Cycle Sequencing Kit according to the manufacturer's instructions. The products of the sequencing reactions were analysed by BIOMI Kft. (Gödöllö, Hungary). Sequences of the oligonucleotides used in section D.2.4.1., are shown in Table 12.

TABLE 12
Name, SEQ ID NO and sequence of oligonucleotides used for constructing genes of proteins
of the present invention for recombinant protein expression

| | | |
|---|---|---|
| NL_3′ | SEQ ID NO: 67 | GAAGTAACGACGTTTCAgCGCACGGCACGGACCCGGGTCgTtTTCCAGGAAGCAGAAGTCC |
| S100A4_seq | SEQ ID NO: 68 | CTCCATCGCCATGATGTCTAACG |
| T7rev | SEQ ID NO: 69 | GCTAGTTATTGCTCAGCGGTGG |
| GLV_5′ | SEQ ID NO: 70 | CAGTGCGAACGTTTCggATACGGTGGTTGCCTGGGTAACATGAACAACTTCGtAACCCTGGAAGAA |
| | | TGC |
| YLV_5′ | SEQ ID NO: 71 | CAGTGCGAACGTTTCtAtTACGGTGGTTGCCTGGGTAACATGAACAACTTCGLAACCCTGGAAGAA |
| | | TGC |
| YFV_5′ | SEQ ID NO: 72 | CAGTGCGAACGTTTCtAtTACGGTGGTTGCtTtGGTAACATGAACAACTTCGLAACCCTGGAAGAA |
| | | TGC |
| YLE_5′ | SEQ ID NO: 73 | CAGTGCGAACGTTTCtAtTACGGTGGTTGCCTGGGTAACATGAACAACTTCGAAACCCTGGAAGAA |
| | | TGC |
| YFE_5′ | SEQ ID NO: 74 | CAGTGCGAACGTTTCtAtTACGGTGGTTGCtTtGGTAACATGAACAACTTCGAAACCCTGGAAGAA |
| | | TGC |
| VRAAAV_3′ | SEQ ID NO: 75 | GGTTGTTGTAGAAGTAACGAacTgcCgcCGCACGGCACacACCCGGGTCGTTTTCC |
| LRAAAV_3′ | SEQ ID NO: 76 | GGTTGTTGTAGAAGTAACGAacTgcCgcCGCACGGCACaGACCCGGGTCGTTTTCC |
| PRAAAV_3′ | SEQ ID NO: 77 | GGTTGTTGTAGAAGTAACGAacTgcCgcCGCACGGCACGGACCCGGGTCGTTTTCC |
| VRALAV_3′ | SEQ ID NO: 78 | GGTTGTTGTAGAAGTAACGAacTgcCAgCGCACGGCACacACCCGGGTCGTTTTCC |
| EVO2a_f | SEQ ID NO: 79 | GACTTCTGCTTCCTGGAAaAtGACCCGGGTCCGTG |
| EVO2a_r | SEQ ID NO: 80 | CACGGACCCGGGTCaTtTTCCAGGAAGCAGAAGTC |
| EVO2b_f | SEQ ID NO: 81 | CAGTGCGAACGTTTCggATACGGTGGTTGCCTG |
| EVO2b_r | SEQ ID NO: 82 | CAGGCAACCACCGTATccGAAACGTTCGCACTG |
| EVO2c_f | SEQ ID NO: 83 | CAGTGCGAACGTTTCtAtTACGGTGGTTGCCTG |
| EVO2c_r | SEQ ID NO: 84 | CAGGCAACCACCGTAaTaGAAACGTTCGCACTG |
| EVO2d_f | SEQ ID NO: 85 | CCGTGCCGTGCGCTGAAACGTCGTTACTTCTAC |
| EVO2d_r | SEQ ID NO: 86 | GTAGAAGTAACGACGTTTCAgCGCACGGCACGG |
| EVO21a_f | SEQ ID NO: 87 | CCGTGCCGTGCGgTGAAACGTCGTTACTTCTAC |
| EVO21a_r | SEQ ID NO: 88 | GTAGAAGTAACGACGTTTCAcCGCACGGCACGG |
| EVO211_f | SEQ ID NO: 89 | GAAAACGACCCGGGTtgGTGCCGTGCGCTGAAAC |
| EVO211_r | SEQ ID NO: 90 | GTTTCAGCGCACGGCACcaACCCGGGTCGTTTTC |
| EVO212_f | SEQ ID NO: 91 | GAAAACGACCCGGGTCtGTGCCGTGCGCTGAAAC |
| EVO212_r | SEQ ID NO: 92 | GTTTCAGCGCACGGCACaGACCCGGGTCGTTTTC |
| EVO213_f | SEQ ID NO: 93 | CCGTGCCGTGCGgcGAAACGTCGTTACTTCTAC |
| EVO213_r | SEQ ID NO: 94 | GTAGAAGTAACGACGTTTCgcCGCACGGCACGG |
| EVO214_f | SEQ ID NO: 95 | GCCGTGCGCTGAAACtTCGTTACTTCTACAACAAC |
| EVO214_r | SEQ ID NO: 96 | GTTGTTGTAGAAGTAACGAaGTTTCAGCGCACGGC |
| EVO215_f | SEQ ID NO: 97 | GAAAACGACCCGGGTtgGTGCCGTGCGgcGAAACGTCGTTACTTCTAC |
| EVO215_r | SEQ ID NO: 98 | GTAGAAGTAACGACGTTTCgcCGCACGGCACcaACCCGGGTCGTTTTC |
| EVO216_f | SEQ ID NO: 99 | GAAAACGACCCGGGTtgGTGCCGTGCGgcGAAACtTCGTTACTTCTACAACAAC |
| EVO216_r | SEQ ID NO: 100 | GTTGTTGTAGAAGTAACGAaGTTTCgcCGCACGGCACcaACCCGGGTCGTTTTC |
| EVO22a_f | SEQ ID NO: 101 | CCGTGCCGTGCGgcGAAACGTCGTTACTTCTAC |
| EVO22a_r | SEQ ID NO: 102 | GTAGAAGTAACGACGTTTCgcCGCACGGCACGG |
| EVO22b_f | SEQ ID NO: 103 | GCCGTGCGCTGgcACGTCGTTACTTCTACAAC |
| EVO22b_r | SEQ ID NO: 104 | GTTGTAGAAGTAACGACGTgcCAGCGCACGGC |

### E.1. Creating the Expression Vectors for the Production of Proteins of the Present Invention

The genes of all exact proteins of the present invention were expressed in the same bacterial expression vector construct applied in the invention described in WO2018127719. The original vector is referred to as pS100A4. Depending on the number and positions of the mutations, the genes of many different proteins of the present invention were constructed by four different ways as described in sections E.1.1.-E.1.4., but in each case the gene was located between the unique BamHI and XhoI sites of the vector. All approaches resulted in the same type of fusion gene construct coding for a fusion protein with the following arrangement:
His6-tag-S100A4 protein-linker peptide-TEV cleavage site-protein of the present invention

The construct enables high-level expression of the fusion proteins in E. coli, purification through immobilized metal ion affinity chromatography through the His6-tag and liberation of the proteins of the present invention by TEV protease (Tobacco Etch Virus protease) processing.

### E.1.1. Creating the Expression Vector for the Production of the Following Variants: EVO21 (SEQ ID NO: 12), EVO22 (SEQ ID NO: 7), EVO23 (SEQ ID NO: 3), EVO24 (SEQ ID NO: 15) and EVO25 (SEQ ID NO: 11)

The five proteins of the present invention mentioned in the title of E.1.2. were produced by a three-step megaprimer mutagenesis method using the pS100A4-EVO2 expression vector containing the gene of the EVO2 (SEQ ID NO: 2) variant, which served as a template.

In the first polymerase chain reaction (PCR) step, the NL_3′ mutagenesis primer (SEQ ID NO: 67) in pair with the S100A4_seq primer (SEQ ID NO: 68) were used for creating a modified EVO2 gene segment, which was to be shared by these proteins of the present invention. The product was purified using the GeneJet PCR Purification Kit. This purified product was used as a forward megaprimer in pair with the T7rev primer (SEQ ID NO: 69) in a reaction using pS100A4-EVO2 as template. The reaction resulted in a modified EVO2 gene carrying mutations in its first half. This PCR was purified using GeneJet PCR Purification Kit and was used in the third PCR step as a template.

In the second PCR step five separate PCRs were conducted. In each reaction there was one variant specific mutagenesis primer (GLV_5′, (SEQ ID NO: 70) for EVO21; YLV_5′ (SEQ ID NO: 71) for EVO22; YFV_5′ (SEQ ID NO: 72) for EVO23; YLE_5′ (SEQ ID NO: 73) for EVO24; and YFE_5′ (SEQ ID NO: 74) for EVO25) that were used in pair with the common T7rev primer (SEQ ID NO: 69). Sequences of the primers are listed in Table 12. The products from the five separate PCRs were individually purified using the GeneJet PCR Purification Kit and were used in the third PCR step as megaprimers.

In the third PCR step, the purified product of the first PCR step was used as template, and the S100A4_seq primer (SEQ ID NO: 67) was used as a forward primer in five separate reactions in pair with one of the five purified megaprimers from the second PCR step generating the final variant genes. These mutant genes were cloned into the pS100A4 fusion expression vector using BamHI and XhoI enzymes as follows. The mutant PCR products and the pS100A4 vector were digested with BamHI (10 U) and XhoI (20 U) in 1× BamHI buffer at 37° C. for 3 hours. The digested DNA products were run on an agarose gel and the fragments of appropriate size were excised and isolated. DNA was eluted from the columns with 30 μl 0.1×EB. The concentrations of the isolated DNA molecules were determined using a BioTek Epoch reader, a Take3 Trio microvolume plate and the Gene5 software. The genes of amino acid sequences according to EVO21 (SEQ ID NO: 12), EVO22 (SEQ ID NO: 7), EVO23 (SEQ ID NO: 3), EVO24 (SEQ ID NO: 15) and EVO25 (SEQ ID NO: 11) were ligated into the vector using T4 DNA ligase. There was 5-fold molar excess of the PCR product in the ligase reaction.

XL1 Blue cells were transformed with the product of the ligase reactions as described in section D.2.1.3.1.1., and spread on an LB/agar+ampicillin (100 μg/ml) plates. The plates were incubated at 37° C. for 16 hours.

Individual colonies of the transformed cells were picked into LB+ampicillin (100 μg/ml) and incubated at 37° C. for 16 hours while shaking at 220 rpm. The plasmid DNA was isolated from the cultures. DNA was eluted from the columns with 50 μl 0.1×EB.

### E.1.2. Creating the Expression Vector for the Production of Proteins According to EVO221 (SEQ ID NO: 21), EVO222 (SEQ ID NO: 18), EVO223 (SEQ ID NO: 19) and EVO224 (SEQ ID NO: 28)

The proteins of the present invention mentioned in the title of E.1.2. were produced by a two-step megaprimer mutagenesis method using the pS100A4-EVO22 expression vector containing the gene of the amino acid sequence according to EVO22 (SEQ ID NO: 7) as template.

In the first PCR step, S100A4_seq primer (SEQ ID NO: 68) was used in pair with one of the four variant-specific mutagenesis primers VRAAAV_3′ (SEQ ID NO: 75), LRAAAV_3′ (SEQ ID NO: 76), PRAAAV_3′ (SEQ ID NO: 77) and VRALAV_3′ (SEQ ID NO: 78) to produce megaprimers for the subsequent step. Sequences of the primers are listed in Table 12. The products from the four separate PCRs were purified using GeneJet PCR Purification Kit.

In the second PCR step, the purified products of the first PCR step were used as forward megaprimer in pair with T7rev primer (SEQ ID NO: 69) with using pS100A4-EVO22 as template. The products of these four separate PCRs were purified using GeneJet PCR Purification Kit.

The mutant genes were cloned into the pS100A4 fusion expression vector using BamHI and XhoI enzymes. The mutant PCR products and the pS100A4 vector were digested with BamHI (10 U) and XhoI (20 U) in 1× BamHI buffer at 37° C. for 3 hours. The digested DNA products were run on an agarose gel and the fragments of appropriate size were excised and isolated. DNA was eluted from the columns with 30 μl 0.1×EB. The concentrations of the isolated DNA molecules were determined using a BioTek Epoch reader, a Take3 Trio microvolume plate and the Gene5 software. The genes of amino acid sequences according to EVO221, (SEQ ID NO: 21), EVO222, (SEQ ID NO: 18), EVO223, (SEQ ID NO: 19) and EVO224, (SEQ ID NO: 28), were ligated into the vector using T4 DNA ligase. There was 5-fold molar excess of the PCR product in the ligase reaction. XL1 Blue cells were transformed with the product of the ligase reactions as described in section D.2.1.3.1.1., and spread on an LB/agar+ampicillin (100 μg/ml) plates. The plates were incubated at 37° C. for 16 hours.

Individual colonies of the transformed cells were picked into LB+ampicillin (100 μg/ml) and incubated at 37° C. for 16 hours while shaking at 220 rpm. The plasmid DNA was isolated from the cultures. DNA was eluted from the columns with 50 μl 0.1×EB.

E.1.3. Creating the Expression Vector for the Production of Proteins According to EVO2a (SEQ ID NO: 32), EVO2b (SEQ ID NO: 31), EVO2c (SEQ ID NO: 25), EVO2d (SEQ ID NO: 30), EVO21a (SEQ ID NO: 24), EVO211 (SEQ ID NO: 4), EVO212 (SEQ ID NO: 14), EVO213 (SEQ ID NO: 27), EVO214 (SEQ ID NO: 8), EVO215 (SEQ ID NO: 26), EVO216 (SEQ ID NO: 29), EVO22a (SEQ ID NO: 6), EVO22b and (SEQ ID NO: 22)

The proteins of the present invention mentioned in the title of E.1.3. were produced by QuikChange mutagenesis method using the pS100A4-EVO2 expression vector containing the gene of the amino acid sequence according to EVO2 (SEQ ID NO: 2) as template (40 ng). The name of the corresponding mutagenesis primer pairs contains the name of the inhibitor variants. For example, the forward and reverse primers for producing EVO2a are named EVO2a_f (SEQ ID NO: 79) and EVO2a_r (SEQ ID NO: 80), respectively. All QuikChange primers are listed in Table 12.

The variants EVO21a (SEQ ID NO: 24), EVO211 (SEQ ID NO: 4), EVO212 (SEQ ID NO: 14), EVO213 (SEQ ID NO: 27), EVO214 (SEQ ID NO: 8), EVO215 (SEQ ID NO: 26) and EVO216 (SEQ ID NO: 29) were produced by QuikChange mutagenesis method using the pS100A4-EVO2 expression vector containing the gene of the amino acid sequence according to EVO21 (SEQ ID NO: 12) as template (40 ng). The corresponding QuikChange primers are listed in Table 12.

The variants EVO22a (SEQ ID NO: 6), and EVO22b (SEQ ID NO: 22) were produced by QuikChange mutagenesis method using the pS100A4-EVO2 expression vectors containing the gene of the amino acid sequence according to EVO22 (SEQ ID NO: 7) as template (40 ng). The corresponding QuikChange primers are listed in Table 12.

The PCRs were carried out in 20 μL using 0.5 μM forward and reverse mutagenesis primers (sequences of the primers being listed in Table 12), 1.25 U KOD polymerase (Sigma-Aldrich) with an annelation temperature of 70° C. and elongation time of 6 min (20 cycles).

The reaction products were treated with 0.5 U DpnI for 1 h at 37° C. to digest the methylated template DNA.

XL1 Blue cells were transformed with the product of the DpnI reactions as described in section 1.3.1.1., and spread on an LB/agar+ampicillin (100 μg/ml) plates. The plates were incubated at 37° C. for 16 hours.

Individual colonies of the transformed cells were picked into LB+ampicillin (100 μg/ml) and incubated at 37° C. for 16 hours while shaking at 220 rpm. The plasmid DNA was isolated from the cultures. DNA was eluted from the columns with 50 μl 0.1×EB.

E.1.4. Creating the Expression Vector for the Production of Proteins According to EVO21b (SEQ ID NO: 9), EVO21c (SEQ ID NO: 13), EVO21d (SEQ ID NO: 16), EVO211a (SEQ ID NO: 20), EVO214a (SEQ ID NO: 17), EVO22d (SEQ ID NO: 10) and EVO23a (SEQ ID NO: 5)

Coding DNA for variants EVO21b (SEQ ID NO: 9), EVO21c (SEQ ID NO: 13), EVO21d (SEQ ID NO: 16), EVO211a (SEQ ID NO: 20), EVO214a (SEQ ID NO: 17), EVO22d (SEQ ID NO: 10) and EVO23a (SEQ ID NO: 5) were purchased as synthetic genes and these were introduced in the pS100A4-EVO2 expression vector by cassette exchange. Sequences of the sense strand of the synthetic genes are named as EVO21b_DNA (SEQ ID NO: 107), EVO21c_DNA (SEQ ID NO: 108), EVO21d_DNA (SEQ ID NO: 109), EVO211a_DNA (SEQ ID NO: 110), EVO214a_DNA (SEQ ID NO: 111), EVO22d_DNA (SEQ ID NO: 112) and EVO23a_DNA (SEQ ID NO: 113) and are listed in Table 13.

TABLE 13
Synthetic genes of EVO21b_DNA (SEQ ID NO: 107),
EVO21c DNA (SEQ ID NO: 108), EVO21d_DNA (SEQ ID NO: 109),
EVO211a DNA (SEQ ID NO: 110), EVO214a_DNA (SEQ ID NO: 111) ,
EVO22d DNA (SEQ ID NO: 112) and EVO23a_DNA (SEQ ID NO: 113)

| | |
|---|---|
| EVO21b_DNA | GGATCCAAACCGGACTTCTGCTTCCTGGAAGAAGACCCGGGTCCGTGCCGTG |
| SEQ ID NO: 107 | CGCTGAAACGTCGTTACTTCTACAACAACCAGACCAAACAGTGCGAACGTTT |
| | CGGATACGGTGGTTGCCTGGGTAACATGAACAACTTCGTAACCCTGGAAGAA |
| | TGCAAAAACATCTGCGAAGACGGTTAATAAGCTTGGCACTCGAG |
| EVO21c_DNA | GGATCCAAACCGGACTTCTGCTTCCTGGAAGAAGACCCGGGTCCGTGCCGTG |
| SEQ ID NO: 108 | CGCTGAAACGTCGTTACTTCTACAACAACCAGACCAAACAGTGCGAACGTTT |
| | CGGATACGGTGGTTGCTTTGGTAACATGAACAACTTCGTAACCCTGGAAGAA |
| | TGCAAAAACATCTGCGAAGACGGTTAATAAGCTTGGCACTCGAG |
| EVO21d_DNA | GGATCCAAACCGGACTTCTGCTTCCTGGAAGAAGACCCGGGTCCGTGCCGTG |
| SEQ ID NO: 109 | CGCTGAAACGTCGTTACTTCTACAACAACCAGACCAAACAGTGCGAACGTTT |
| | CAGCTACGGTGGTTGCTTTGGTAACATGAACAACTTCGTAACCCTGGAAGAA |
| | TGCAAAAACATCTGCGAAGACGGTTAATAAGCTTGGCACTCGAG |
| EVO211a_DNA | GGATCCAAACCGGACTTCTGCTTCCTGGAAGAAGACCCGGGTTGGTGCCGTG |
| SEQ ID NO: 110 | CGCTGAAACGTCGTTACTTCTACAACAACCAGACCAAACAGTGCGAACGTTT |
| | CGGATACGGTGGTTGCTTTGGTAACATGAACAACTTCGTAACCCTGGAAGAA |
| | TGCAAAAACATCTGCGAAGACGGTTAATAAGCTTGGCACTCGAG |
| EVO214a_DNA | GGATCCAAACCGGACTTCTGCTTCCTGGAAGAAGACCCGGGTCCGTGCCGTG |
| SEQ ID NO: 111 | CGCTGAAACTGCGTTACTTCTACAACAACCAGACCAAACAGTGCGAACGTTT |
| | CGGATACGGTGGTTGCTTTGGTAACATGAACAACTTCGTAACCCTGGAAGAA |
| | TGCAAAAACATCTGCGAAGACGGTTAATAAGCTTGGCACTCGAG |
| EVO22d_DNA | GGATCCAAACCGGACTTCTGCTTCCTGGAAGAAGACCCGGGTCCGTGCCGTG |
| SEQ ID NO: 112 | CGGCGAAACGTCGTTACTTCTACAACAACCAGACCAAACAGTGCGAACGTTT |
| | CTATTACGGTGGTTGCTTTGGTAACATGAACAACTTCGTAACCCTGGAAGAA |
| | tGCAAAAACATCTGCGAAGACGGTTAATAAGCTTGGCACTCGAG |
| EVO23a_DNA | GGATCCAAACCGGACTTCTGCTTCCTGGAAGAAGACCCGGGTCCGTGCCGTG |
| SEQ ID NO: 113 | CGCTGAAACGTCGTTACTTCTACAACAACCAGACCAAACAGTGCGAACGTTT |
| | CTATTACGGTGGTTGCTTTGGTAACATGAACAACTTCGTAACCCTGGAAGAA |
| | TGCAAAAACATCTGCGAAGACGGTTAATAAGCTTGGCACTCGAG |

The mutant genes were cloned into the pS100A4 fusion expression vector using BamHI and XhoI enzymes. The mutant PCR products and the pS100A4-EVO2 vector were digested with BamHI (10 U) and XhoI (20 U) in 1× BamHI buffer at 37° C. for 3 hours. The digested DNA products were run on an agarose gel and the fragments of appropriate size were excised and isolated. DNA was eluted from the columns with 30 μl 0.1×EB. The concentrations of the isolated DNA molecules were determined using a BioTek Epoch reader, a Take3 Trio microvolume plate and the Gene5 software. The DNA fragment according to EVO221_DNA, EVO222_DNA, EVO223_DNA, and EVO224_DNA were ligated into the vector using T4 DNA ligase. There was 5-fold molar excess of the PCR product in the ligase reaction.

XL1 Blue cells were transformed with the product of the ligase reactions as described in section D.2.1.3.1.1., and spread on an LB/agar+ampicillin (100 μg/ml) plates. The plates were incubated at 37° C. for 16 hours.

Individual colonies of the transformed cells were picked into LB+ampicillin (100 μg/ml) and incubated at 37° C. for 16 hours while shaking at 220 rpm. The plasmid DNA was isolated from the cultures. DNA was eluted from the columns with 50 μl 0.1×EB.

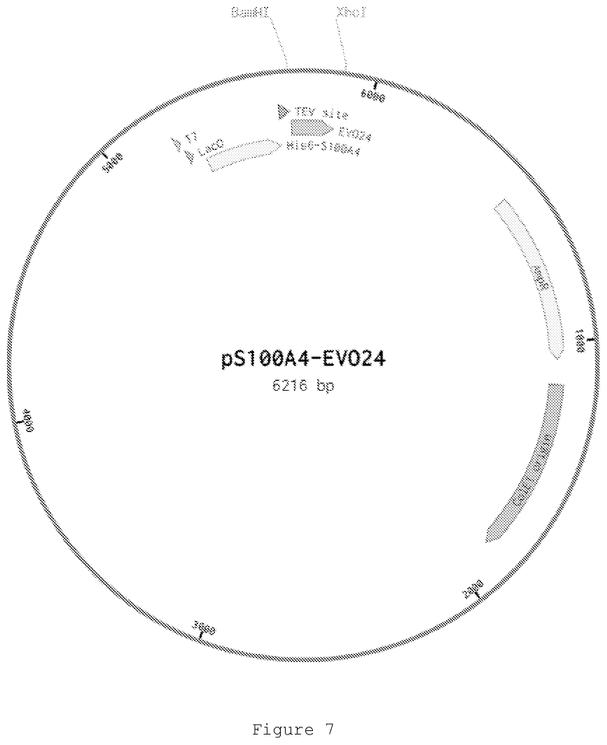

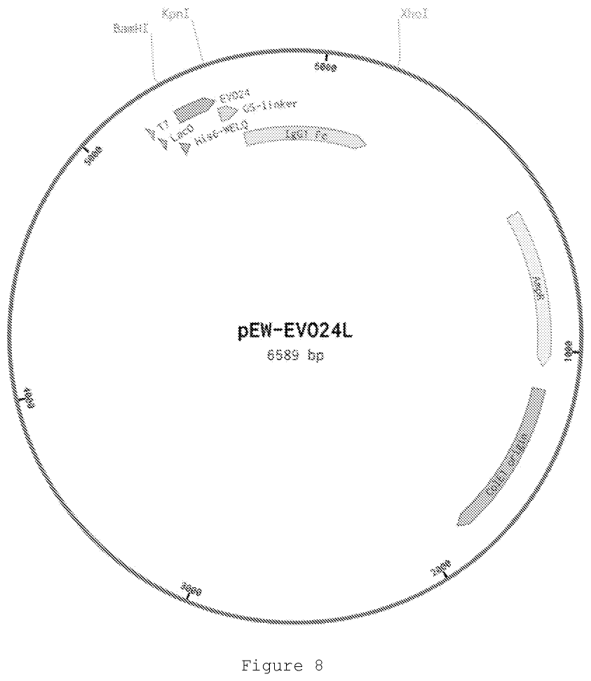

The expression vector construct used for the production of all twenty-nine single domain proteins of the present invention is illustrated in FIG. 7, using the example of EVO24.

### E.2. Bacterial Production of the Recombinant Proteins

We used E. coli Shuffle B (NEB, C3028J) for protein expression. This strain was engineered to allow the formation of disulfide bridges in the cytoplasm. It also expresses the disulfide bond isomerase and chaperone protein DsbC in the cytoplasm to help protein folding by assisting in the formation of the most stable native disulfide bridge pattern (Lobstein 2012).

### E.2.1 Transformation

1 μl expression vector and 100 μl Shuffle B competent cell were used. The cells were incubated on ice for 30 minutes, and then for 1 minute they were exposed to a heat shock at 42° C. 200 μl LB medium was added to the cells, it was shaken for 30 minutes at 37° C., and then it was spread on an LB/agar+ampicillin (100 μg/ml) plate. The plate was incubated overnight at 30° C.

### E.2.2 Biomass Production

Cells on the plate were washed into 30 ml LB+ampicillin (75 μg/ml) and shaken at 30° C. overnight to serve as the starter culture. 1 L Terrific Broth (TB) medium (12 g Tripton, 24 g Yeast Extract, 4 ml glycerol dissolved in 900 mL water+100 mL phosphate buffer containing 0.72 M K₂HPO₄ and 0.17 M KH₂PO₄ is added) was poured into 2.8 l Fernbach flasks and supplemented with ampicillin to 75 μg/ml final concentration. The flasks were pre-incubated at 30° C. while shaking at 180 rpm, then the starter culture was added, and the flasks were shaken until the cultures reached an OD_{600 nm} value of 0.8. At this point, the expression of the recombinant gene was induced by adding IPTG solution to a final concentration of 0.1 mM, and the cultures were shaken for additional 16-20 hours at 18° C. Then, the cells were pelleted by centrifugation (5 minutes, 7,500×g, 4° C.), the supernatant was discarded, the wet weight of the cell pellet was determined and the cells were suspended in an appropriate volume of 50 mM Tris-HCl, 500 mM NaCl buffer to reach a cell pellet wet weight/cell suspension volume ratio of 1 g/5 mL.

### E.2.3 Protein Purification

The cells were disrupted by sonication and the samples were centrifuged to remove the cell debris (20 minutes, 48,000×g). The supernatant containing the fusion protein and other soluble components of the cytoplasm was loaded onto an IMAC column (10 ml BioRad Profinity IMAC resin) containing immobilized nickel ions. The column was equilibrated with a 50 mM Tris-HCl, 500 mM NaCl pH 8.0 buffer (IMAC buffer). After loading the sample on the column, the column was washed with 10 column volume of IMAC buffer. His-tagged S100A4-fused inhibitors were eluted with 50 mM Tris-HCl, 250 mM imidazole, 300 mM NaCl, pH 8.0 buffer (IMAC elution buffer).

The eluted fusion protein was dialyzed against 20 mM Tris-HCl pH 8.0, 150 mM NaCl (dialysis buffer) for 3 hours at room temperature in order to reduce the concentration of imidazole in the sample using dialysis tubing cellulose membrane with a cut-off value of 12-14 kDa (Sigma—D9527).

### E.2.4 Proteolytic Processing

TEV protease cleavage (the protease was added in a molar ratio of 1:50-1:100) took place in a buffer containing 20 mM Tris-HCl pH 8.0, 150 mM NaCl, 1 mM reduced and 0.1 mM oxidized glutathione.

The reaction was incubated at room temperature for 16 hours. His-tagged version of the TEV protease was produced in house based on the publication of van den Berg, 2006, with modifications and the purified enzyme was stored at −80° C. in the presence of 1 mM TCEP. The extent of cleavage was tested by SDS PAGE on 15% Tris-Tricine gel.

### E.2.5 Isolation of the Proteins of the Present Invention

At this stage of the procedure, major components of the sample are the processed proteins (lacking the His-tag), His6-tagged S100A4, His6-tagged TEV protease and possibly some unprocessed fusion protein. The sample was centrifuged to remove any precipitations and reloaded onto the IMAC column equilibrated with dialysis buffer. The His6-tagged protein components of the sample were captured by the immobilized nickel ions of the resin while the processed protein was in the flow through fraction.

The flow through fraction was collected and was dialyzed against 150 mM NH₄-acetate (gel filtration buffer) for 3 hours at room temperature using dialysis tubing cellulose membrane with a cut-off value of 3.5 kDa (Thermo—68035). After lyophilisation, the proteins of the present invention were resuspended in gel filtration buffer and loaded onto Superdex 30 HiLoad 16/60 column equilibrated with gel filtration buffer. The main peak was collected. Molar concentration of samples of proteins of the present invention was determined based on UV absorption at 280 nm, the samples were distributed in aliquots, lyophilized and stored at 4° C.

### E.2.6 Isolation of the Proteins of the Present Invention for In Vivo Studies

For samples prepared for in vivo studies, prior to the gel filtration step, a cation exchange chromatography step was introduced in order to further minimize nucleic acid and endotoxin contamination. The flow-through fraction of the second IMAC step, containing the proteins of the present invention, was dialyzed against 8 mM NH₄-acetate/42 mM acetic acid pH 4.0 buffer, and the sample was loaded onto a 5 mL HiTrap SP HP cation exchange chromatography column (15 mL/min) (Cytiva 17115201) equilibrated with the same buffer. After extensive wash with dialysis buffer (15 mL/min), the proteins of the present invention were eluted with the gradient created by switching to 150 mM NH₄-acetate, i.e., the gel filtration buffer (8 CV, 2 mL/min).

After lyophilisation, the proteins of the present invention were resuspended in gel filtration buffer and loaded onto Superdex 30 HiLoad 16/60 column equilibrated with gel filtration buffer. The main peak was collected. Molar concentration of samples of proteins of the present invention was determined based on UV absorption at 280 nm, and the samples were distributed in aliquots, lyophilized and stored at 4° C.

### E.3. Developing an Fc-Fusion Version of EVO24 (SEQ ID NO: 15) Referred to as EVO24L (SEQ ID NO: 114).

It is apparent for the person skilled in the art that the Kunitz domain proteins of the present invention can be combined with another protein, or a part of another protein such that the Kunitz domain protein maintains its functions related to the invention, but in the context of the chimeric protein predictably gains new or enhanced beneficial properties provided by the other protein part.

One of the most widely known example of this approach is furnishing a peptide or protein according to an invention with an antibody Fc-domain. This Fc-domain can be natural or modified and can provide various predictable properties through protein-protein interactions. Such predictable properties are strictly governed by the amino acid sequence and the presence or lack of various posttranslational modifications of the particular Fc domain. For example, some Fc domains can form stable monomers, homodimers, heterodimers or larger multimers, some can bind to the neonatal Fc-receptor protein (FcRn), which results in extended in vivo half-life of the chimera protein, some can bind to other host proteins, which mediate various functional consequences such as classical complement pathway activation or immune cell activation etc. It is known to the person skilled in the art that bacterial expression results in non-glycosylated proteins and that lack of Fc-glycosylation does not compromise the half-life extending property of such Fc domains, but eliminates most of the immune-activation effector functions.

In this example we applied the above principles by developing a chimeric protein containing EVO24 (SEQ ID NO: 15) of this invention fused to an IgG1-type Fc domain that forms stable homodimers. The protein is referred to as EVO24L (SEQ ID NO: 114).

### E.3.1. DNA Construct Enabling EVO24L (SEQ ID NO: 114) Expression

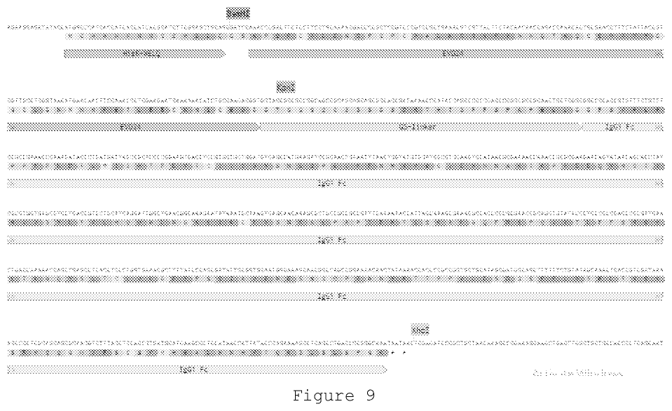

A starting version of an EVO24-Fc domain fusion encoding DNA was purchased as synthetic gene and inserted into a bacterial expression vector providing an N-terminal His-tag coding sequence that can be cut off by the WELQut (also known as SplB) protease. In this DNA construct designed restriction enzyme sites allowed for easy replacement of the linker between the N-terminal EVO24 Kunitz segment and the C-terminal Fc domain as well as a segment within the Fc domain affecting the monomeric/dimeric nature of the domain. Starting from this synthetic DNA construct we used simple recombinant DNA methods to develop the optimized construct introduced in this example. Relevant part of the final DNA construct and the sequence of the encoded protein are shown in FIG. 8 and FIG. 9, respectively.

Amino acid sequence of the WELQut-processed form of EVO24L corresponds to SEQ ID NO: 114.

### E.3.2. Bacterial Production and Isolation of EVO24L

Protocols for production and isolation of EVO24L (SEQ ID NO: 114) were very similar to those detailed in the corresponding sections of E.2. therefore here only the differences are described. Proteolytic removal of the His-tag was performed with a His-tagged WELQut protease produced by us as a recombinant protein. Unlike TEV protease, the WELQut enzyme does not require reducing conditions for its activity, therefore the proteolytic processing can be carried out at normal oxidising environment, which is ideal for processing disulfide containing proteins. After the second IMAC step, the pH of the flow-through fraction containing processed EVO24L (SEQ ID NO: 114) was adjusted to 7.0 by 1 M NaH₂PO₄ buffer and the sample was loaded to a 50 mL Cytiva HiTrap Q HP anion-exchange resin (#17101401) containing XK 26/20 column equilibrated with the same composition buffer as the pH-adjusted dialysis buffer (AEX buffer A). The column was washed with AEX buffer. At this pH EVO24L flows through while the majority of the contaminations including nucleic acids and other endotoxins are captured by the column. The AEX flow-through was concentrated by Pierce™ Protein Concentrator PES, 10K MWCO, 5-20 mL (#88527) and the concentrated sample was loaded on a Cytivia HiLoad 26/600 Superdex 200 size exclusion column equilibrated with the vehicle buffer containing 25 mM Na-phosphate pH 6.3, 100 mM NaCl and 2.5% wt/vol sucrose. The EVO24L containing peak was collected and the samples were concentrated again on new Pierce™ Protein Concentrator PES, 10K MWCO, 5-20 mL (#88527) tubes. The concentration of the sample was determined based on (ε₂₈₀=41.995 M⁻¹cm⁻¹) and was adjusted to 1 mM. The sample was tested by analytical gel filtration and C4 deposition serum assay for lectin pathway inhibitory efficacy.

### E.3.3. Lectin Pathway Inhibitory Efficacy of EVO24L (SEQ ID NO: 114)

As described in 5.4.1., IC₅₀ values of the EVO24L sample were determined. The inner standard was the EVO24 (SEQ ID NO: 15). For the dimeric EVO24L, the molar concentration of the Kunitz domain part was considered. The IC₅₀ values of EVO24L were 2.5-fold higher than that of EVO24 suggesting that the Kunitz domain part is fully functional, but in the given test only one Kunitz domain can bind at a time to the immobilized MASP-2 target.

### Example F: Functional Characterisation of the Inhibitors

### F.1. LC-MS Analysis of the Proteins of the Present Invention

Verification of the proper molecular weight of the proteins of the present invention was done by mass spectrometric experiments performed on a high-resolution hybrid quadrupole-time-of-flight mass spectrometer (Waters Select Series Cyclic IMS, Waters Corp., Wilmslow, U.K.). The mass spectrometer operated in positive W mode. Leucine enkephalin was used as Lock Mass standard. ESI ionization was performed using a ZSpray ion source operated under the following parameters: capillary voltage: 2 kV, cone gas flow: 20 L/h, desolvation gas flow: 800 L/h, desolvation temperature: 400° C., nebulizer gas: 6 bar, source temperature: 120° C. Chromatographic separations were performed on a Waters Acquity I-Class UPLC system, coupled directly to the mass spectrometer. RPLC-MS analysis were performed on a Waters Acquity BEH300 C4 UPLC column (2.1×150 mm, 1.7 μm) under the following parameters: mobile phase “A”: 0.1% trifluoroacetic acid in water, mobile phase “B”: 0.1% trifluoroacetic acid in acetonitrile; flow rate: 400 μL/min; column temperature: 80° C.; gradient: 2 min: 5% B, 8 min: 45% B, 8.5 min: 90% B, 9 min: 90% B, 9.1 min: 5% B, 12 min: 5% B. UV detection was performed at 220 and 280 nm. The m/z range was 350-2000. Data acquisition and analysis was performed by the MassLynx 4.2 software. The mass accuracy exceeded 5 ppm.

### F.2. Determining K_{I} Constants on Human and Rat MASP-2

The equilibrium inhibition constant (K₁) of nine proteins of the present invention designed and produced based on the first and second stages of the directed evolution campaign was measured on human MASP-2 (5 nM) and rat MASP-2 (2 nM). These variants are: EVO21 (SEQ ID NO: 12), EVO22 (SEQ ID NO: 7), EVO23 (SEQ ID NO: 3), EVO24 (SEQ ID NO: 15), EVO25 (SEQ ID NO: 11), EVO221 (SEQ ID NO: 21), EVO222 (SEQ ID NO: 18), EVO223 (SEQ ID NO: 19) and EVO224 (SEQ ID NO: 28).

For determining the K₁ of the proteins of the present invention on MASP enzymes we used catalytic enzyme fragments containing the three C-terminal domains: CCP1-CCP2-SP. The synthetic substrate used in the measurements was Z-L-Lys-SBzl hydrochloride (Sigma, C3647), from which a 10 mM stock solution was prepared. The reactions were performed in a volume of 0.2 ml at room temperature in 20 mM HEPES; 145 mM NaCl; 5 mM CaCl₂); 0.05% Triton-X100 pH 7.4 buffer. The free thiol generated upon enzyme catalysed hydrolysis of the thioester bound in the substrate reacted with the auxiliary substrate, 5,5′-Dithiobis(2-nitrobenzoic acid) (DTNB, Ellman's reagent, Sigma—D8130) present in the solution in 2-fold excess to Z-L-Lys-SBzl. This results in the release of a chromogenic group, which was monitored through the increase of absorbance at 410 nm using a BioTek Synergy H4 multimode microplate reader.

Serial dilutions were prepared from the individual inhibitors, and after adding the enzyme, the complex formation was allowed to proceed for 2 hours at room temperature. The samples were transferred on a 96-well microtiter plate (Nunc 269620). The reactions were started by adding the mixture of the substrate and the auxiliary substrate to the samples in 250 μM and 500 μM final concentration, respectively. Substrate concentration and data collection time were optimized such that it ensured lower than 10% substrate consumption, i.e., a practically constant rate of product formation within the timeframe of the measurement. For determining the K_{I} values, a method developed for the characterisation of tight-binding inhibitors (Empie 1982) which was modified later (Szakács 2019) was used. The rate of product formation was determined by measuring the change of absorbance, which is a linear function of the product concentration, as a function of reaction time.

Reaction rates determined for inhibitor-containing samples were divided by reaction rates corresponding to samples that contain no inhibitor. This ratio was multiplied with the total enzyme concentration to obtain the free enzyme concentration in the inhibitor containing samples. These free enzyme concentration data were then plotted as a function of the total inhibitor concentration and the K_{I} value was calculated by non-linear fitting to the following equation:

$$E = y = {E}_{0} - ( {E}_{0} + x + Ki - {( ( {( {E}_{0} + x + Ki )}^{⋀} 2 ) - 4 * {E}_{0} * x )}^{⋀} ( 1 / 2 ) ) / 2 ,$$

where E is the free enzyme concentration, and E₀ is the total enzyme concentration. The stock concentration of the inhibitors was determined by titration with bovine trypsin of known concentration. The results were calculated as the average of at least two parallel measurements. The results are summarised in Table 14 below.

TABLE 14
Equilibrium inhibition constant values of proteins
of the present invention on human and rat MASP-2.

K_{I} values (nM)

| Inhibitor | human MASP-2cf | rat MASP-2cf |
|---|---|---|
| EVO2 | 0.65 ± 0.49 | 0.24 ± 0.06 |
| SEQ ID NO: 2 | | |
| EVO21c | 0.065 ± 0.0065 | 0.007 ± 0.003 |
| SEQ ID NO: 13 | | |
| EVO22 | 0.15 ± 0.17 | 0.31 ± 0.09 |
| SEQ ID NO: 7 | | |
| EVO24 | 0.34 ± 0.03 | 0.36 ± 0.14 |
| SEQ ID NO: 15 | | |
| EVO25 | 0.17 ± 0.04 | 0.9 ± 1.17 |
| SEQ ID NO: 11 | | |
| EVO221 | 0.19 ± 0.11 | 0.1 ± 0.1 |
| SEQ ID NO: 21 | | |
| EVO222 | 0.1 ± 0.2 | 0.27 ± 0.05 |
| SEQ ID NO: 18 | | |
| EVO223 | 0.17 ± 0.07 | 0.09 ± 0.04 |
| SEQ ID NO: 19 | | |
| EVO224 | 1.13 ± 0.83 | 0.14 ± 0.15 |
| SEQ ID NO: 28 | | |

The results demonstrated that from the nine proteins of the present invention, eight were more potent human MASP-2 inhibitors than EVO2 (SEQ ID NO: 2). In fact, for these eight proteins the binding was too tight to be accurately measured. Therefore an independent method, surface plasmon resonance (SPR) was used.

### F.3. Surface Plasmon Resonance Based Kinetic Parameters and Affinity Values of Proteins of the Present Invention on Human and Rat MASP-2

To accurately determine the affinities of the tightest binding inhibitors according to present invention to human and rat MASP-2 catalytic fragments, the method of surface plasmon resonance spectroscopy was applied using Bio-Rad ProteOn™ XPR36 Protein Interaction Array System.

Based on the K_{I} values of the nine proteins of the present invention mentioned above, thirteen additional proteins were designed, produced and isolated. Some of these were designed to better understand the sequence to activity relationships of the proteins while others to identify additional ultra-high efficiency MASP-2 inhibitors.

The previous nine and the new thirteen proteins, therefore altogether twenty-two proteins of the present invention were tested along with EVO2 by the SPR method. The list of the twenty-two proteins is the following: EVO21 (SEQ ID NO: 12), EVO22 (SEQ ID NO: 7), EVO23 (SEQ ID NO: 3), EVO24 (SEQ ID NO: 15), EVO25 (SEQ ID NO: 11), EVO221 (SEQ ID NO: 21), EVO222 (SEQ ID NO: 18), EVO223 (SEQ ID NO: 19) and EVO224 (SEQ ID NO: 28), EVO2a (SEQ ID NO: 32), EVO2b (SEQ ID NO: 31), EVO2c (SEQ ID NO: 25), EVO2d (SEQ ID NO: 30), EVO21a (SEQ ID NO: 24), EVO211 (SEQ ID NO: 4), EVO212 (SEQ ID NO: 14), EVO213 (SEQ ID NO: 27), EVO214 (SEQ ID NO: 8), EVO215 (SEQ ID NO: 26), EVO216 (SEQ ID NO: 29), EVO22a (SEQ ID NO: 6), and EVO22b (SEQ ID NO: 22).

With the SPR method, in addition to the binding affinity, the kinetics of complex formation and complex dissociation were also quantitatively assessed delivering association rate coefficient (kₒₙ) and dissociation rate coefficient (k_{off}) values.

Human or rat MASP-2 catalytic fragments were covalently immobilized onto a Bio-Rad ProteOn™ GLC Sensor Chip (15 μg/mL in 10 mM Na-acetate pH 4.5) to a ligand density of 2500 RU. The inhibitors were injected onto the chip in two-fold serial dilutions across five points complemented with a buffer control using the running buffer containing 20 mM HEPES pH 7.4, 150 mM NaCl, 2 mM CaCl₂), 0.5 mM MgCl₂, 0.005% Tween-20, 6 mM NaN₃. Kinetic coefficients were obtained by the global fitting of double referenced association and dissociation phases using the 1:1 Langmuir model. To control the reproducibility of the measurements EVO2 (SEQ ID NO: 2) was injected regularly in the course of the experiment as an inner reference. The relative standard deviation of both K_{d} and Rₘₐₓ for repetitive measurements of EVO2 was acceptable (<30%). The results are summarized in Table 15.

TABLE 15
SPR-based kinetic and affinity values of the proteins of the present invention

| | Human | | | | Rat | | | |
|---|---|---|---|---|---|---|---|---|
| | kon | koff | KD | Relative | kon | koff | KD | Relative |
| Variant | (1/Ms) | (1/s) | (pM) | affinity | (1/Ms) | (1/s) | (pM) | affinity |
| EVO2 | 8.09E+05 | 1.40E−03 | 1790 | 1.0 | 3.10E+05 | 1.07E−03 | 3630 | 1.0 |
| EVO21 | 1.32E+06 | 4.92E−05 | 37 | 48.0 | 5.45E+05 | 4.49E−05 | 82 | 44.1 |
| EVO214 | 1.27E+06 | 4.78E−05 | 38 | 47.6 | 6.14E+05 | 7.19E−05 | 117 | 31.0 |
| EVO211 | 1.54E+06 | 6.62E−05 | 43 | 41.6 | 9.42E+05 | 2.70E−03 | 2870 | 1.3 |
| EVO22a | 2.04E+06 | 9.09E−05 | 45 | 40.1 | 8.47E+05 | 1.63E−04 | 192 | 18.9 |
| EVO222 | 9.54E+05 | 4.26E−05 | 45 | 40.0 | 4.63E+05 | 2.62E−04 | 566 | 6.4 |
| EVO223 | 8.82E+05 | 4.77E−05 | 54 | 33.1 | 6.96E+05 | 7.53E−05 | 108 | 33.6 |
| EVO212 | 1.51E+06 | 8.26E−05 | 55 | 32.7 | 4.92E+05 | 6.18E−04 | 1260 | 2.9 |
| EVO23 | 2.10E+06 | 1.97E−04 | 94 | 19.1 | 9.82E+05 | 6.62E−04 | 674 | 5.4 |
| EVO221 | 1.03E+06 | 1.31E−04 | 127 | 14.1 | 6.64E+05 | 1.13E−04 | 170 | 21.4 |
| EVO22 | 2.30E+06 | 3.14E−04 | 137 | 13.1 | 1.12E+06 | 2.56E−04 | 229 | 15.9 |
| EVO25 | 2.16E+06 | 4.38E−04 | 203 | 8.8 | 1.05E+06 | 8.26E−04 | 787 | 4.6 |
| EVO22b | 1.24E+06 | 2.60E−04 | 210 | 8.5 | 9.75E+05 | 1.59E−04 | 163 | 22.3 |
| EVO24 | 2.25E+06 | 6.22E−04 | 276 | 6.5 | 1.13E+06 | 3.27E−04 | 289 | 12.6 |
| EVO215 | 1.84E+06 | 5.59E−04 | 304 | 5.9 | nd | nd | nd | nd |
| EVO213 | 9.67E+05 | 5.24E−04 | 542 | 3.3 | 3.86E+05 | 1.47E−03 | 3810 | 1.0 |
| EVO224 | 1.10E+06 | 6.17E−04 | 561 | 3.2 | 8.39E+05 | 1.84E−04 | 219 | 16.6 |
| EVO21a | 1.16E+06 | 8.39E−04 | 723 | 2.5 | 5.15E+05 | 1.04E−03 | 2020 | 1.8 |
| EVO216 | 9.63E+05 | 7.32E−04 | 760 | 2.4 | nd | nd | nd | nd |
| EVO2c | 1.55E+06 | 1.61E−03 | 1040 | 1.7 | 7.47E+05 | 9.78E−04 | 1310 | 2.8 |
| EVO2d | 9.09E+05 | 1.08E−03 | 1190 | 1.5 | 4.05E+05 | 4.38E−04 | 1080 | 3.4 |
| EVO2b | 7.90E+05 | 1.56E−03 | 1970 | 0.9 | 4.84E+05 | 9.42E−04 | 1950 | 1.9 |
| EVO2a | 7.11E+05 | 2.10E−03 | 2950 | 0.6 | 3.19E+05 | 1.51E−03 | 4730 | 0.8 |

### F.4. Effects of Proteins of the Present Invention on the Three Complement-Activation Pathways in Human Serum

### F.4.1. Inhibitory Potency of the Proteins of the Present Invention on the Human Lectin Pathway

As outlined above, the complement system can be activated through three pathways, which converge at the level of C3 convertases. The three activation pathways are the classical, the lectin and the alternative pathway. MASP-1 and MASP-2 are lectin pathway specific proteases and both are key enzymes in lectin pathway activation. Complete inhibition of any of these proteases completely block the lectin pathway activation. The protein inhibitors of the present invention were therefore expected to block the lectin pathway activation while not affecting the other two pathways or the convertase enzymes of the common complement route.

The so-called WIELISA kit (Euro-Diagnostica AB, COMPL300) was developed for selective measurement of the activation of the three complement pathways. The kit applies three different conditions, each ensuring that only one of the three pathways can be activated, while the other two remain inactive. The kit detects the latest emerging component of complement activation on the route where the three pathways already merged: a neo-epitope of C9 in the C5-9 complex. However, Kocsis et al. developed another assay for the same purpose (Kocsis 2010). This assay follows the principles of the WIELISA kit. The activation of the pathways can be measured by detecting the deposition of activated C3 or C4 fragments, or the C5-9 neo-epitope through antibodies specific to the above-mentioned complement components. This method was used for assessing the inhibitory potency of the proteins of the present invention as MASP-2 inhibitors.

The assay was performed using normal human serum (Quidel Corporation, A113). A 5 ml aliquot was thawed on ice, distributed to aliquots, and stored at −80° C. until use.

The assay was performed as described by Kocsis et al. (Kocsis 2010), with modifications. 96-well Greiner high binding ELISA plates (cat. no. 655061) were coated with 100 μl/well 10 μg/ml mannan dissolved in coating buffer (50 mM sodium-carbonate pH 9.6) overnight at 4° C. Control wells contained coating buffer alone. Wells were blocked for at least 1 h at 37° C. with 200 μl/well 10 mg/ml bovine serum albumin (BSA) dissolved in 50 mM Tris pH 7.4, 150 mM NaCl buffer.

Normal human serum was thawed on ice and diluted with 20 mM HEPES pH 7.4, 5 mM CaCl₂), 5 mM MgCl₂, 150 mM NaCl, 0.1% Tween-20 (serum dilution buffer). The dilution of the serum was 25-fold. Serial dilutions of the inhibitors were made in serum dilution buffer and were added to the diluted serum samples to reach final serum dilutions 50-fold. The samples were incubated for 30 minutes at room temperature.

The ELISA plate was washed thoroughly with 50 mM Tris pH 7.4, 5 mM CaCl₂), 150 mM NaCl, 0.1% Tween-20 (washing buffer) and then the pre-incubated serum samples were transferred to the plate. Two negative controls were made. In one, the diluted serum containing no inhibitor was transferred on surfaces treated only with BSA. In the other negative control, the diluted serum was transferred onto mannan coated surfaces, but was supplemented with EDTA (ethylenediaminetetraacetic acid) to a final concentration of 20 mM. EDTA prevents all Ca²⁺ and Mg²⁺ ion dependent “downstream” complement activation steps via chelating these ions. A 50-fold diluted serum containing no inhibitor was also transferred to the mannan-coated plate to assess maximal complement activity. The plate was incubated at 37° C. for 30 minutes, washed with the washing buffer and 100-100 μl of α-human C4c antibody (rabbit) (DakoCytomation—Q0369) diluted 3000-fold in wash buffer containing 10 mg/ml BSA (antibody buffer) was pipetted into the wells. The plate was incubated at 37° C. for 60 minutes. The plate was washed again and 100 μl/well peroxidase conjugated α-rabbit IgG monoclonal antibody (mouse) (Sigma—A1949) diluted 40,000-fold in antibody buffer was transferred to the plate and the plate was incubated for 30 minutes at 37° C. The plate was rinsed again with washing buffer. Then, 100 μl/well 1 mg/ml o-phenylenediamine dihydrochloride (OPD, Sigma—P9029) peroxidase substrate dissolved in 50 mM citrate pH 5.0, 0.1% H₂O₂ buffer was transferred to the plate to generate a photometric signal proportionate to the amount of C4 deposited onto the surface. After signal development, the reaction was stopped by adding 50 μl/well 1 M sulfuric acid. The 490 nm absorbance values were recorded using a BioTek Synergy H4 Hybrid reader. Three parallels were measured for each data point. The 0% serum activity was represented by the serum samples containing 20 mM EDTA, while 100% activity was represented by the serum samples having no inhibitor added.

The data were analysed with Origin Pro 8 software, and the inhibitor concentration providing 50% C4 deposition inhibition (IC₅₀) was determined by fitting the DoseResp function (Pharmacology built-in equation set) onto the data. In this experiment, the complement inhibitory efficacy of the proteins of the present invention were compared to that of EVO2. All measurements were performed at the same time and on the same plate, from one single thawed serum sample, and assessed the IC₅₀ of the proteins of the present invention in comparison to the IC₅₀ value of EVO2 determined on the same plate.

Because IC₅₀ values might depend on the actual serum sample used, the data were normalised through the following steps:
i) IC₅₀ value of the examined variant was expressed as a fraction of the IC₅₀ value of the EVO2 in the given experiment.
ii) An average of the IC₅₀ values of the EVO2 from the measurements was calculated and was termed as the “average IC₅₀” of EVO2.
iii) The IC₅₀ values of the examined proteins of the present invention were re-calculated as the fraction (step “i”) of the average IC₅₀ and were termed as “normalised IC₅₀”.

Through this normalisation process, we were able to directly compare the inhibitory potency of each protein of the present invention to each other and determine the most potent lectin pathway inhibitors. The results are presented in Table 16.

The data demonstrated that further evolved proteins of the present invention are up to 48-fold more efficient inhibitors of the human lectin pathway than EVO2. The IC₅₀ values of the most efficient variants are in the 2-10 nM range.

TABLE 16
Lectin pathway inhibitory potency of
proteins of the present invention

| | | |
|---|---|---|
| Variant | Human | Rat |

| | SEQ | IC50 | Relative | IC50 | Relative |
|---|---|---|---|---|---|
| Name | ID NO | (nM) | potency | (nM) | potency |
| EVO2 | 2 | 138.0 | 1.0 | 106.0 | 1.0 |
| EVO23 | 3 | 2.9 | 47.1 | 8.1 | 13.1 |
| EVO211 | 4 | 3.1 | 44.0 | 53.2 | 2.0 |
| EVO23a | 5 | 3.2 | 43.0 | 14.1 | 7.5 |
| EVO22a | 6 | 3.7 | 37.1 | 10.6 | 10.0 |
| EVO22 | 7 | 4.1 | 33.4 | 9.9 | 10.8 |
| EVO214 | 8 | 4.3 | 32.4 | 11.2 | 9.5 |
| EVO21b | 9 | 4.3 | 31.9 | 7.2 | 14.7 |
| EVO22d | 10 | 5.6 | 24.7 | 10.4 | 10.2 |
| EVO25 | 11 | 5.6 | 24.5 | 15.0 | 7.1 |
| EVO21 | 12 | 5.7 | 24.1 | 10.9 | 9.8 |
| EVO21c | 13 | 6.2 | 22.4 | 12.4 | 8.5 |
| EVO212 | 14 | 6.2 | 22.1 | 46.3 | 2.3 |
| EVO24 | 15 | 7.6 | 18.1 | 6.6 | 16.2 |
| EVO21d | 16 | 8.3 | 16.6 | 25.9 | 4.1 |
| EVO211a | 20 | 9.1 | 15.2 | 30.6 | 3.5 |
| EVO222 | 18 | 9.8 | 14.1 | 24.2 | 8.9 |
| EVO223 | 19 | 9.9 | 14.0 | 12.0 | 8.9 |
| EVO214a | 17 | 10.2 | 13.6 | 20.3 | 5.2 |
| EVO221 | 21 | 12.7 | 10.9 | 11.0 | 9.7 |
| EVO22b | 22 | 13.7 | 10.0 | 9.7 | 11.0 |
| EVO21a | 24 | 21.4 | 6.5 | 39.4 | 2.7 |
| EVO2c | 25 | 22.3 | 6.2 | 34.3 | 3.1 |
| EVO215 | 26 | 22.7 | 6.1 | nd. | nd. |
| EVO213 | 27 | 28.3 | 4.9 | 88.7 | 1.2 |
| EVO216 | 29 | 30.9 | 4.5 | nd. | nd. |
| EVO224 | 28 | 30.9 | 4.5 | 29.6 | 3.6 |
| EVO2d | 30 | 60.3 | 2.3 | 70.9 | 1.5 |
| EVO2b | 31 | 62.7 | 2.2 | 34.3 | 3.1 |
| EVO2a | 32 | 215.0 | 0.6 | 213.0 | 0.5 |

### F.4.2. Assessing the Effects of the Proteins of the Present Invention on Human Classical and Alternative Pathway Activation

Based on high inhibitory potency against both human and rat lectin pathway activation, the following four proteins of the present invention were selected to test their pathway specificity: EVO21 (SEQ ID NO: 12), EVO214 (SEQ ID NO: 8), EVO23 (SEQ ID NO: 3), and EVO24 (SEQ ID NO: 15). The assays were carried out similarly as described in F.4.1., with the following modifications: i) For selective classical pathway activation, 100 μl/well 10 μg/ml aggregated human IgG was immobilized onto the ELISA plates. For the activation of the alternative pathway, 100 μl/well 100 μg/ml zymosan (from Saccharomyces cerevisiae) was immobilized. ii) The final dilution of the serum was 60-fold in the classical pathway measurements and 6-fold in the alternative pathway measurements.

For the alternative pathway measurements 20 mM HEPES pH 7.4, 5 mM MgCl₂, 20 mM EGTA, 150 mM NaCl, 0.1% Tween-20 was used instead of the serum dilution buffer. The EGTA [ethylene glycol-bis(β-aminoethyl ether)-N,N,N′,N′-tetraacetic acid] component chelates the calcium ions specifically preventing the classical and lectin pathway activation, which, unlike the alternative pathway, are calcium ion dependent. iii) The activity of the pathways was assessed via using α-human anti C3c antibody (rabbit) (DakoCytomation—A0062) in 2000-fold dilution for the classical and 5000-fold dilution for the alternative pathway.

In the first experiment the proteins of the present invention were tested at fixed 10 μM concentration. The negative and positive controls were prepared as described in 5.4.1. The four proteins of the present invention exerted from small to moderate inhibition on the classical and negligible inhibition on the alternative pathway at a concentration three orders of magnitude higher than their lectin pathway inhibitory IC₅₀ values. This demonstrates that these proteins of the present invention are lectin pathway specific. The results are shown in Table 17.

TABLE 17
Effects of four selected proteins of the present
invention on human classical and alternative complement
pathway activation at 10 μM concentration.

| | |
|---|---|
| | Pathway activity at 10 μM |
| Name and | inhibitor concentration |

| SEQ ID NO | Classical | Alternative |
|---|---|---|
| EVO21 | 67.7% | 97.8% |
| SEQ ID NO: 12 | | |
| EVO214 | 98.6% | 90.9% |
| SEQ ID NO: 8 | | |
| EVO23 | 86.4% | 96.8% |
| SEQ ID NO: 3 | | |
| EVO24 | 97.1% | 86.8% |
| SEQ ID NO: 15 | | |

A lack of classical pathway and alternative pathway inhibition demonstrates that these proteins of the present invention do not inhibit the following seine proteases: C1r, C1s, factor D, factor B (i.e., the C3bBb type C3-convertase) and C2 (i.e., the C4b2a type C3-convertase), verifying high target specificity of EVO21 (SEQ ID NO: 12), EVO214 (SEQ ID NO: 8), EVO23 (SEQ ID NO: 3), and EVO24 (SEQ ID NO: 15).

### F.5. Effects of Proteins of the Present Invention on the Three Complement-Activation Pathways in Rat Serum

### F.5.1. Inhibitory Potency of the Proteins of the Present Invention on Rat Lectin Pathway

Efficacy of the proteins of the present invention in inhibiting the lectin pathway in rat serum was carried out essentially as described for the human lectin pathway in F.4.1., but two modifications were implemented: the rat serum was used in 60-fold dilution, and the antibody detecting the deposited C4 fragments was diluted 2000-fold. The α-human C4c antibody (DakoCytomation—Q0369) recognizes rat C4 fragments. Pooled rat serum was used. Evaluation of the data was as described in F.4.1.

All, except of two proteins of the present invention, namely EVO215 (SEQ ID NO: 26) and EVO216 (SEQ ID NO: 29) are potent inhibitors of the lectin pathway in rat serum. The results are shown in Table 16 in Example F, section F.4.1.

### F.5.2. Assessing the Effects of Proteins of the Present Invention on Rat Classical and Alternative Pathway Activation

Assessment of the specificity of the four selected proteins of the present invention was carried out as described in F.4.2., for the human serum. Results are shown in Table 18.

TABLE 18
Effects of four selected proteins of the present
invention on rat classical and alternative complement
pathway activation at 10 μM concentration.

| | |
|---|---|
| | Pathway activity at 10 μM |
| Name and | inhibitor concentration |

| SEQ ID NO | Classical | Alternative |
|---|---|---|
| EVO21 | 59.8% | 65.2% |
| SEQ ID NO: 12 | | |
| EVO214 | 88.8% | 26.1% |
| SEQ ID NO: 8 | | |
| EVO23 | 86.7% | 12.2% |
| SEQ ID NO: 3 | | |
| EVO24 | 89.6% | 98.0% |
| SEQ ID NO: 15 | | |

Because the proteins EVO214 (SEQ ID NO: 8) and EVO23 (SEQ ID NO: 3) showed significant inhibitory effect against the alternative pathway in rat serum, an assay with serial dilution of the two inhibitors were carried out in order to determine the IC₅₀ values of these inhibitors. The method is described in F.4.2., the results are listed in Table 19 and are discussed at the end of this section.

TABLE 19
EVO214 (SEQ ID NO: 8) and EVO23 (SEQ ID NO: 3) inhibit the
rat alternative pathway only at high concentrations.

| | Residual activity of the alternative pathway at the indicated | | | | | | |
|---|---|---|---|---|---|---|---|
| | inhibitor concentrations in 6-fold diluted rat serum | | | | | | |
| | 0.1 μM | 0.5 μM | 1 μM | 2 μM | 3 μM | 5 μM | 10 μM |
| Evo 23 | 85.8% | 87.5% | 88.1% | 87.7% | 100.8% | 76.7% | 16.8% |
| SEQ ID NO: 3 | | | | | | | |
| Evo 214 | 92.2% | 103.2% | 107.2% | 102.8% | 104.1% | 94.1% | 61.1% |
| SEQ ID NO: 8 | | | | | | | |

While EVO23 (SEQ ID NO: 3) provides over 80% alternative pathway in rat serum, it does so only at a high, 10 μM concentration, where EVO214 (SEQ ID NO: 8) provides only 39% inhibition. Neither proteins exert significant inhibition on the alternative pathway up to 5 μM concentration.

In all, the proteins of the present invention are potent inhibitors of the lectin pathway in rat serum, with IC₅₀ values being in the 10⁻⁷-10⁻⁹ M range. The four chosen proteins, EVO21 (SEQ ID NO: 12), EVO214 (SEQ ID NO: 8), EVO23 (SEQ ID NO: 3), and EVO24 (SEQ ID NO: 15) do not inhibit the classical pathway in rat serum, and exert inhibitory effect against the alternative pathway only at high, 10 micromolar concentrations. As this inhibitory effect is observed in 6-times diluted serum, it can be deduced that this effect becomes even less significant in vivo in whole blood. Taking these into account, the four inhibitors can be considered as specific lectin pathway inhibitors in rat serum.

A lack of significant classical pathway and alternative pathway inhibition demonstrates that these four selected proteins of the present invention do not inhibit the following serine proteases: C1r, C1s, factor D, factor B (i.e., the C3bBb type C3-convertase) and C2 (i.e., the C4b2a type C3-convertase), verifying high target specificity of EVO21 (SEQ ID NO: 12), EVO214 (SEQ ID NO: 8), EVO23 (SEQ ID NO: 3), and EVO24 (SEQ ID NO: 15).

### F.6. Effect of Proteins of the Present Invention on Human and Rat Blood Coagulation

The effect of the proteins of the present invention on the blood coagulation process was tested in three standard assays, the thrombin time, testing any direct effects on thrombin; prothrombin time, testing any effects on the extrinsic pathway; and the activated partial thromboplastin time, testing any effects on the intrinsic pathway.

### F.6.1. Human Blood Coagulation Measurements

Blood was collected from a healthy individual by vein puncture after informed consent. The blood was treated with sodium-citrate (3.8% wt/vol) and centrifuged. All three assays were performed on the automated instrument Sysmex CA-1500 (Sysmex) with Innovin reagent (Dale Behring, Marburg, Germany).

The proteins of the present invention were applied in a twofold serial dilution in 1.4% wt/vol sodium bicarbonate (vehicle) with the highest final concentration being 10 μM (3.4 μM for EVO24 (SEQ ID NO: 15)) and the lowest final concentration being 156 nM (53 nM for EVO24 (SEQ ID NO: 15)).

All measurements were done in duplicates and a vehicle control was also tested. The highest concentration value is about 5 orders of magnitudes higher than the K_{D} values of the four proteins of the present invention on human MASP-2.

The results are summarized in Tables 20a-20d.

Tables 20: Effects of EVO21 (SEQ ID NO: 12), EVO24 (SEQ ID NO: 15), EVO23 (SEQ ID NO: 3), and EVO214 (SEQ ID NO: 8) on the human blood coagulation.

TABLE 20a

| EVO21 | PI | PI | APTT | APTT | TT | TT |
|---|---|---|---|---|---|---|
| (μM) | 21/1 | 21/2 | 21/1 | 21/2 | 21/1 | 21/2 |
| 10 | 11.4 | 11.9 | 58.7 | 67.6 | 16.8 | 16.4 |
| 5 | 10.6 | 10.3 | 45.6 | 40.1 | 16.5 | 17.4 |
| 2.5 | 10.3 | 10.2 | 40.0 | 37.1 | 16.4 | 17.4 |
| 1.25 | 10.2 | 10.1 | 37.0 | 34.8 | 16.9 | 17.1 |
| 0.625 | 10.1 | 10.0 | 34.3 | 33.6 | 16.8 | 16.9 |
| 0.3125 | 10.1 | 10.0 | 33.6 | 32.8 | 16.9 | 17.0 |
| 0.15625 | 10.2 | 10.0 | 32.9 | 32.3 | 17.1 | 17.0 |
| 0 | 10.2 | 10.0 | 31.9 | 32.0 | 17.2 | 17.0 |

TABLE 20b

| EVO24 | PI | PI | APTT | APTT | TT | TT |
|---|---|---|---|---|---|---|
| (μM) | 24/1 | 24/2 | 24/1 | 24/2 | 24/1 | 24/2 |
| 3.4 | 10.8 | 10.8 | 39.6 | 40.2 | 16.8 | 16.7 |
| 1.7 | 10.4 | 10.3 | 35.8 | 35.9 | 16.8 | 17.0 |
| 0.85 | 10.2 | 10.1 | 34.0 | 34.4 | 16.7 | 16.8 |
| 0.425 | 10.0 | 10.0 | 33.0 | 33.1 | 16.8 | 17.0 |
| 0.2125 | 10.0 | 9.9 | 32.7 | 33.1 | 17.0 | 17.2 |
| 0.10625 | 10.0 | 10.0 | 32.1 | 32.4 | 16.8 | 16.7 |
| 0.053125 | 10.0 | 9.9 | 32.1 | 32.2 | 16.8 | 16.8 |
| 0 | 10.0 | 9.9 | 31.8 | 32.0 | 16.7 | 16.8 |

TABLE 20c

| EVO23 | PI | PI | APTT | APTT | TT | TT |
|---|---|---|---|---|---|---|
| (μM) | 23/1 | 23/2 | 23/1 | 23/2 | 23/1 | 23/2 |
| 10 | 13.4 | 13.5 | 86.6 | 90.2 | 17.3 | 16.9 |
| 5 | 11.9 | 11.8 | 57.9 | 56.4 | 17.0 | 16.6 |
| 2.5 | 11.2 | 11.4 | 46.0 | 46.6 | 16.8 | 16.9 |
| 1.25 | 10.7 | 10.7 | 40.5 | 40.4 | 16.5 | 16.7 |
| 0.625 | 10.4 | 10.5 | 36.7 | 36.9 | 16.7 | 16.8 |
| 0.3125 | 10.2 | 10.1 | 34.8 | 34.7 | 16.9 | 16.7 |
| 0.15625 | 10.2 | 10.1 | 33.6 | 33.5 | 16.8 | 16.8 |
| 0 | 10.0 | 9.9 | 31.6 | 31.8 | 16.7 | 16.9 |

TABLE 20d

| EVO214 | PI | PI | APTT | APTT | TT | TT |
|---|---|---|---|---|---|---|
| (μM) | 214/1 | 214/2 | 214/1 | 214/2 | 214/1 | 214/2 |
| 10 | 10.7 | 10.7 | 70.7 | 71.1 | 16.7 | 17.2 |
| 5 | 10.2 | 10.2 | 54.2 | 53.8 | 16.5 | 16.9 |
| 2.5 | 10.0 | 10.0 | 47.0 | 46.1 | 16.9 | 17.0 |
| 1.25 | 10.0 | 10.0 | 41.1 | 41.3 | 16.9 | 17.3 |
| 0.625 | 9.9 | 9.9 | 37.3 | 37.9 | 17.1 | 17.3 |
| 0.3125 | 9.9 | 9.9 | 35.4 | 35.1 | 16.9 | 17.2 |
| 0.15625 | 10.0 | 9.9 | 33.4 | 33.8 | 16.8 | 17.3 |
| 0 | 10.0 | 10.0 | 32.6 | 33.4 | 16.8 | 17.0 |

### F.6.2. Rat Blood Coagulation Measurements

The rat blood coagulation assays were measured on a Sysmex CA-660 Coagulation analyser using blood plasma of Wistar rats. All proteins of the present invention were tested on two plasma aliquots of three rats. The proteins of the present invention were dissolved in 1.4% wt/vol sodium bicarbonate vehicle. EVO24 (SEQ ID NO: 15) was tested at 3.4 μM, while EVO21 (SEQ ID NO: 12), EVO214 (SEQ ID NO: 8) and EVO23 (SEQ ID NO: 3) at 10 μM plasma concentration.

The results are listed in Tables 21.

TABLES 21
Effects of EVO214 (SEQ ID NO: 8), EVO21 (SEQ
ID NO: 12), EVO23 (SEQ ID NO: 3), and EVO24
(SEQ ID NO: 15) on the rat blood coagulation.

| | | APTT | PT | TT |
|---|---|---|---|---|
| Assay | | [sec] | [sec] | [sec] |
| EVO214 | Mean | 30.68 | 9.88 | 31.45 |
| (10 μM) | SD | 0.35 | 0.05 | 3.93 |
| | N | 4 | 4 | 4 |
| Vehicle | Mean | 13.34 | 9.52 | 32.18 |
| | SD | 1.47 | 0.18 | 5.18 |
| | N | 5 | 5 | 5 |
| EVO21 | Mean | 33.53 | 10.10 | 23.92 |
| (10 μM) | SD | 2.37 | 0.17 | 0.75 |
| | N | 6 | 6 | 6 |
| Vehicle | Mean | 13.88 | 9.53 | 29.72 |
| | SD | 2.14 | 0.14 | 2.42 |
| | N | 6 | 6 | 6 |
| EVO23 | Mean | 32.35 | 10.30 | 28.42 |
| (10 μM) | SD | 2.07 | 0.11 | 0.70 |
| | N | 6 | 6 | 6 |
| Vehicle | Mean | 13.13 | 9.55 | 28.43 |
| | SD | 2.22 | 0.10 | 0.76 |
| | N | 6 | 6 | 6 |
| EVO24 | Mean | 36.08 | 9.53 | 40.97 |
| (3.4 μM) | SD | 9.98 | 0.18 | 14.80 |
| | N | 5 | 6 | 6 |
| Vehicle | Mean | 19.02 | 9.43 | 45.10 |
| | SD | 3.78 | 0.12 | 18.78 |
| | N | 5 | 6 | 6 |

APTT stands for activated partial thromboplastin time, PT stands for prothrombin time, and TT stands for thrombin time in the three standard blood coagulation tests.

### F.6.3. Conclusions of the Blood Coagulation Tests

Even at the highest concentration, the proteins of the present invention had no or negligible effect in the PT and TT tests both in human and rat assays. On the basis of the results it can be clearly stated about the proteins of the present invention that they do not inhibit the following blood coagulation proteases with considerable affinity: thrombin, fVIIa and fXa.

On the other hand, all these four proteins of the present invention lengthened the APTT time at or above 1 μM concentration, which is about 3-4 orders of magnitudes higher than the K_{D} values of the four proteins of the present invention on human and rat MASP-2.

Nevertheless, effects on the APTT indicated that these proteins of the present invention can at least weakly inhibit at least one of the following blood coagulation enzymes: fIXa, fXIa and fXIIa.

Therefore these proteins of the present invention were also tested in vitro in these enzymes.

F.7. Testing the Efficacy of EVO21 (SEQ ID NO: 12), EVO214 (SEQ ID NO: 8), EVO23 (SEQ ID NO: 3), and EVO24 (SEQ ID NO: 15) of the Present Invention on MASP-1 and on Human Blood Coagulation Factors fIXa, fXIa and fXIIa

As newest studies show that MASP-1 also contributes to the physiologic coagulation of human blood (Golomingi 2022), we also tested the four compounds for MASP-1 inhibitory potency.

The measurements were done using non-binding microtiter plates (Greiner; #655901) in 100 μL final volume. After some pilot experiments to find the proper inhibitor concentration range, the maximal inhibitor concentration was set to 20 μM for MASP-1, 10 μM for fXIa and 40 μM for fIXa and fXIIa inhibition, and twofold serial dilutions were prepared from the proteins of the present invention. To these solutions, the proteins of the present invention were added to reach a previously optimized final concentration, 10 nM for MASP-1, 50 nM for fIXa, 3.3 nM for fXIa and 27.5 nM for fXIIa.

The samples were incubated for 10 minutes at room temperature. After adding the substrate (150 μM Z-Lys-S-Benzyl and 300 μM 4,4′-dithiodipyridine (DTDP) co-substrate for MASP-1; 150 μM Z-Gly-Arg-S-Benzyl and 300 μM DTDP co-substrate for fIXa; 300 μM Cbz-GPR-pNA for fXIa and 100 μM H-D-PFR-pNA for fXIIa). The reaction buffer was 50 mM Tris-HCl, 150 mM NaCl, 5 mM CaCl₂), 0.1% PEG-8000 pH 7.4 for MASP-1; 20 mM HEPES, 145 mM NaCl, 0.05% Triton-X100 pH 7.4 for fIXa and fXIa; which was complemented with 5 mM CaCl₂) for fXIIa.

The efficacies of the proteins of the present invention were determined in the form of (IC₅₀) values corresponding to inhibitor concentrations that provide 50% inhibition. The results are summarized in Table 22.

TABLE 22
IC₅₀ values of EVO21, EVO214, EVO23
and EVO24 on the indicated proteases.

IC₅₀ (μM)

| Inhibitor | MASP-1 | FIXa | FXIa | FXIIa |
|---|---|---|---|---|
| EVO21 | 13 | 28 | 0.8 | 10.5 |
| SEQ ID NO: 12 | | | | |
| EVO214 | 9.6 | 24 | 0.2 | 3.7 |
| SEQ ID NO: 8 | | | | |
| EVO23 | 6.5 | 2.6 | 0.5 | 3.3 |
| SEQ ID NO: 3 | | | | |
| EVO24 | 9.5 | 123 | 0.5 | 16.1 |
| SEQ ID NO: 15 | | | | |

In all, the four selected proteins of the present invention inhibit MASP-1 with IC₅₀ values around 10 μM, fIXa and fXIIa in the 3-100 μM range, which are negligibly weak inhibitions. Only fXIa is inhibited with sub-micromolar IC₅₀, which is still around 3-4 orders of magnitude higher than the K_{D} values of the four proteins of the present invention on human and rat MASP-2.

Nevertheless, a potential off-target effect on fXIa could be expected. It is important to note that complete deficiency of fXI is associated with minor bleeding upon trauma. Yet, besides fXIIa, fXIa is also considered as an optimal target for developing new antithrombotic drugs that are safer than the presently available compounds (Mohammed 2018), (Al-Horani 2016).

### F.8. Measurements of the Stability of the Proteins of the Present Invention

For assessing the in vivo lectin pathway inhibitory capacity of the four selected proteins, EVO21 (SEQ ID NO: 12), EVO23 (SEQ ID NO: 3), EVO24 (SEQ ID NO: 15) and EVO214 (SEQ ID NO: 8), we aimed to store and use them at high, 1.5 mM concentration in 1.4% wt/vol sodium bicarbonate buffer that is a proper vehicle in animal experiments. For testing the stability of these four proteins, lyophilized samples from the same batch were compared. One sample was dissolved in 1.4% sodium bicarbonate buffer and stored at 4° C. for 5 weeks. The other aliquot sample was freshly dissolved into vehicle buffer. Lectin pathway inhibitory potency of these samples was assessed at the same time on the same plate as described in F.5.1.

No significant difference was found between the IC₅₀ values of the freshly dissolved and the 5-week-old samples demonstrating that the proteins of the present invention are stable for at least five weeks in vehicle buffer.

Stability of EVO24L (SEQ ID NO: 114) was tested the same way by determining the IC₅₀ values of a freshly prepared EVO24L samples and EVO24L samples stored at 4° C. for 5 weeks. No significant difference was found between the IC₅₀ values of the freshly prepared and 5-week-old samples demonstrating that EVO24L is stable for at least 5 weeks in vehicle buffer.

### F.9. Monitoring the Extent and Time Course of In Vivo Complement Lectin Pathway Inhibition Efficacy of the Proteins of the Present Invention Administered to Rats by Determining Residual Lectin Pathway Activity of Serum Samples Ex Vivo.

Two such campaigns were conducted. In the first campaign EVO21 (SEQ ID NO: 12), EVO24 (SEQ ID NO: 15), EVO23 (SEQ ID NO: 3) and EVO214 (SEQ ID NO: 8) of the present invention were tested in comparison to EVO2 (SEQ ID NO: 2). In the second campaign, an Fc-fusion form of EVO24 referred to as EVO24L (SEQ ID NO: 114) was tested. The description below under F.9.1., details the general procedure with specific data corresponding to the first campaign. Specific differences in the second campaign are explained in section F.9.2.

### F.9.1. Pharmacodynamic Testing of EVO21 (SEQ ID NO: 12), EVO23 (SEQ ID NO: 3), EVO24 (SEQ ID NO: 15) and EVO214 (SEQ ID NO: 8)

Healthy male Wistar-Hanover rats (4 animals/test protein) were anesthetized by intraperitoneal (ip) injection of pentobarbital sodium and repeated doses to maintain anaesthesia. A heating pad assured maintenance of body temperature. The ECG of the animals (leads I-II-III) was monitored during the experiments. In the event of spontaneous breathing cessation or a significant reduction in heart rate or cardiac arrhythmias, the animals were intubated per os and connected to a rodent ventilator (Ugo Basile, Model 7025, Varese, Italy) for artificial ventilation at a rate and a stroke volume according to the manufacturer's recommendations. After development of anaesthesia, blood samples were taken by cannulation of the right carotid artery. Arterial blood was collected through a polyethylene cannula, directly dripped into tubes containing coagulation activator (VACUETTE® TUBE 2 ml Z Serum Clot Activator).

Test substances (1.5 mM) or vehicle were administered as a slow intravenous (iv) bolus injection lasting 1 min, followed immediately by an intraperitoneal bolus injection at a dose volume of 1.5 mL/kg the two doses together corresponding to 4.5 μmol/kg and about 32 mg/kg dose. Blood samples were taken 20 min and 5 min before administration of test substances or vehicle and 5, 30, 60, 120, and 240 min after administration of test substance or vehicle for serum sample preparation. The blood samples were incubated at room temperature for 30±5 minutes to allow blood coagulation. The coagulated blood samples were centrifuged at 4000 rpm at 20° C. for 15 minutes. Two 100 μl serum samples were taken from each blood sample and precisely pipetted into a 0.5 ml Eppendorf tube. Serum aliquots were frozen in liquid nitrogen and stored at −80° C. until assaying.

Activity of the complement lectin pathway activity was assessed by activation of the serum samples on a mannan-coated surface and measurement of C4b deposition by ELISA measurement. Briefly, the surface of the microtiter plates was covered with mannan (10 μg/ml) overnight at 4° C. After washing, wells were blocked with 1% BSA solution to prevent non-specific protein binding. The rat serum samples were applied in a 32-fold dilution to the surface covered with mannan and incubated for 30 minutes at 37° C. During this time, MASP-2 is activated and cleaves the C4 component, and the C4b fragment covalently deposits on the surface of the microtiter plate.

In the case of a 32-fold serum dilution, the activation of the lectin pathway takes place efficiently in the rat serum, but the alternative activation pathway is not initiated. Although activation of the alternative pathway would not contribute to the measured signal, because it does not generate cleaved C4, if the alternative pathway would activate, high density of the excessive amount of deposited C3b component could compete for the free surface with C4b.

After washing, the primary antibody (anti-human C4c polyclonal rabbit antibody (DakoCytomation—Q0369) was applied at first to the surface of the plates in 2,000-fold dilution. After another washing, an anti-rabbit IgG horseradish peroxidase (HRP) conjugated monoclonal mouse antibody (Sigma—A1949) was used at 40,000-fold dilution. The HRP component of the conjugated antibody catalysed the chemical reaction between the OPD (Ortho-Phenylenediamine) peroxidase and hydrogen-peroxide substrates of the enzyme at a rate proportional to the amount of immobilized HRP. The enzyme reaction was allowed to proceed for 8 minutes at room temperature, then, it was stopped with adding 1M sulfuric acid solution. The absorbance was read at a wavelength of 490 nm using a spectrophotometer.

As the absorbance is proportional to the amount of HRP, which is itself proportional to the deposited C4b, we could infer the level of activity of the lectin pathway. Serum samples obtained from each animal were assayed in triplicates on the same plate. When reading the pharmacodynamic (PD) endpoint, the 100% (control serum) activity value was given by the lectin pathway activity of the serum without the protein of the present invention (pre-dose sample taken at −5 min), while the 0% value (subtracted background absorbance at complete inhibition) was provided by the control serum treated with EDTA. For measurement of the inhibitory effects of administered test substances (i.e., proteins of the present invention) or vehicle, the C4b deposition values measured in serum samples taken at different time points were expressed in percentage of the background-subtracted absorbance value of −5 min pre-dose sample from the same rat. These percent lectin pathway activation values were subjected to descriptive statistical evaluation and plotting.

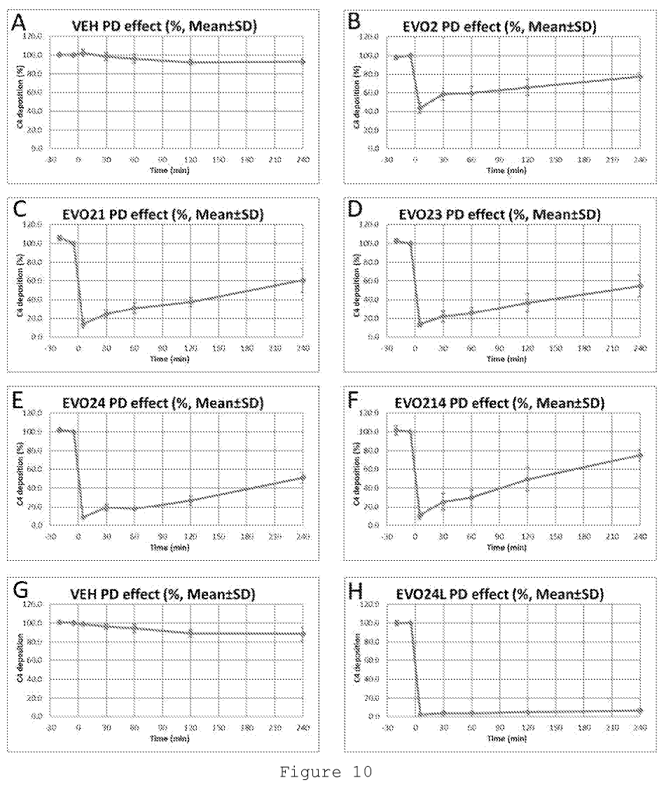

As FIG. 10 illustrates, based on the extent of maximal lectin pathway inhibitory capacity at 5 minutes after administration, EVO21, EVO23, EVO24 and EVO214 are all significantly more efficient in vivo lectin pathway inhibitors than EVO2. While at the applied those of 32 mg/kg, EVO2 can provide 56% pathway inhibition, the four tested proteins of the present invention provide 86-91% inhibition. The most efficient protein is EVO24, which after 1 hour provides 80% and after 4 hours about 50% inhibition. The corresponding data of EVO2 are 40% and 20% inhibition, respectively.

Based on these data we produced an Fc-fusion version of EVO24 to decrease its clearance rate from the blood and thereby improve its pharmacodynamic properties.

### F.9.2. Pharmacodynamic Testing of EVO24L (SEQ ID NO: 114)

For EVO24L the procedure was the same as described in F.9.1., except that the entire 4.5 μmol/kg dose was administered to the rats as a slow intravenous (iv) bolus injection lasting 2 min and no intraperitoneal administration was applied. In this case 5 animals/proteins (vehicle or EVO24L) were used.

As FIG. 10 demonstrates, the Fc fusion of EVO24L dramatically enhanced the efficacy of the compound. At the first sampling time, 5 minutes, EVO24L provided over 98% lectin pathway inhibition, and even at the last, 4-hour time point, lectin pathway inhibition was about 95%. Note that the fact that C4 deposition was completely inhibited clearly demonstrates that non-glycosylated nature of the IgG1 fusion tag prevents IgG-dependent activation of the classical complement pathway.

In all, due to the Fc fusion tag, a single dose of EVO24L can provide almost complete pathway inhibition for at least hours. It clearly demonstrates that with repeated doses or slow-release devices complete and long term lectin pathway blockade can be achieved in this type of example of the present invention.

### LITERATURE REFERENCES

Al-Horani, R. A. and Desai, U. R. (2016) Factor XIa inhibitors: A review of the patent literature. Expert Opin. Ther. Pat. 26, 323-345.
Ali Y M, Ferrari M, Lynch N J, Yaseen S, Dudler T, Gragerov S, Demopulos G, Heeney J L, Schwaeble W J. (2021) Lectin Pathway Mediates Complement Activation by SARS-CoV-2 Proteins. Front Immunol. 12:714511.
Ambrus, G., Gál, P., Kojima, M., Szilágyi, K., Balczer, J., Antal, J., Graf, L., Laich, A., Moffatt, B., Schwaeble, W., Sim, R. B. and Závodszky, P. (2003) Natural substrates and inhibitors of mannan-binding lectin-associated serine protease-1 and -2: a study on recombinant catalytic fragments. J. Immunol. 170, 1374-1382.
Ammitzboll, C. G., Thiel, S., Ellingsen, T., Deleuran, B., Jorgensen, A., Jensenius, J. C. and Stengaard-Pedersen, K. (2012) Levels of lectin pathway proteins in plasma and synovial fluid of rheumatoid arthritis and osteoarthritis. Rheumatol. Int. 32, 1457-1463.
Asgari, E., Farrar, C. A., Lynch, N., Ali, Y. M., Roscher, S., Stover, C., Zhou, W., Schwaeble, W. J. and Sacks, S. H. (2014) Mannan-binding lectin-associated serine protease 2 is critical for the development of renal ischemia reperfusion injury and mediates tissue injury in the absence of complement C4. FASEB J. 28(9):3996-4003.
Castellano, G., Melchiorre, R., Loverre, A., Ditonno, P., Montinaro, V., Rossini, M., Divella, C., Battaglia, M., Lucarelli, G., Annunziata, G., Palazzo, S., Selvaggi, F. P., Staffieri, F., Crovace, A., Daha, M. R., Mannesse, M., van Wetering, S., Paolo Schena, F. and Grandaliano, G. (2010) Therapeutic targeting of classical and lectin pathways of complement protects from ischemia-reperfusion-induced renal damage. Am. J. Pathol. 176, 1648-59.
Chan R K, Ding G, Verna N, Ibrahim S, Oakes S, Austen W G Jr, Hechtman H B, Moore F D Jr. (2004) IgM binding to injured tissue precedes complement activation during skeletal muscle ischemia-reperfusion. J Surg Res. 122(1):29-35.
Clark J E, Dudler T, Marber M S, Schwaeble W. (2018) Cardioprotection by an anti-MASP-2 antibody in a murine model of myocardial infarction. Open Heart. 5(1):e000652.
Collard C D, Vakeva A, Morrissey M A, Agah A, Rollins S A, Reenstra W R, Buras J A, Meri S, Stahl G L. (2000) Complement activation after oxidative stress: role of the lectin complement pathway. Am J Pathol. 156(5):1549-56.
Conway E M (2015) HUS and the case for complement. Blood. 126(18):2085-90.
Crooks G E, Hon G, Chandonia J M, Brenner S E. (2004) WebLogo: a sequence logo generator. Genome Res. 14 1188-1190.
Cummings W J, Wood C, Yabuki M, Li Y, Dudler T, Yaseem S, et al. (2017) MASP-3 Antibody Treatment Blocks Pro-Df Maturation, Reduces AP Activity, and Prevents Collagen Antibody-Induced Arthritis. Late-Breaking Abstract. 16th European Meeting on Complement in Human Disease, Copenhagen.
Damoah C E, Snir O, Hindberg K, Garred P. Ludviksen J K, Brækkan S K, Morelli V M, Eirik Mollnes T, Hansen J B; INVENT Consortium*. (2022) High Levels of Complement Activating Enzyme MASP-2 Are Associated With the Risk of Future Incident Venous Thromboembolism. Arterioscler Thromb Vasc Biol. 21:101161ATVBAHA122317746.
Davis A E 3rd, Lu F, Mejia P. (2010) C1 inhibitor, a multi-functional serine protease inhibitor. Thromb Haemost. 104(5):886-93.
Debreczeni M L, Németh Z, Kajdácsi E, Schwaner E, Makó V, Masszi A, Doleschall Z, Rigó J, Walter F R, Deli M A, Pál G, Dobó J, Gál P, Cervenak L. (2019) MASP-1 Increases Endothelial Permeability. Front Immunol. 10:991.
Degn S E, Kjaer T R, Kidmose R T, Jensen L, Hansen A G, Tekin M, Jensenius J C, Andersen G R, Thiel S. (2014) Complement activation by ligand-driven juxtaposition of discrete pattern recognition complexes. Proc Natl Acad Sci USA. 111(37):13445-50.
Dézsi L, Mészáros T, Kozma G, H-Velkei M, Oláh C Z, Szabó M, Patkó Z, Fülöp T, Hennies M, Szebeni M, Barta B A, Merkely B, Radovits T, Szebeni J. (2022) A naturally hypersensitive porcine model may help understand the mechanism of COVID-19 mRNA vaccine-induced rare (pseudo) allergic reactions: complement activation as a possible contributing factor. Geroscience. 44(2):597-618.
Diebolder C A, Beurskens F J, de Jong R N, Koning R I, Strumane K, Lindorfer M A, Voorhorst M, Ugurlar D, Rosati S, Heck A J R, et al. (2014) Complement is activated by IgG hexamers assembled at the cell surface. Science 343:1260-1263.
Dobó J, Kocsis A, Gál P. (2018) Be on Target: Strategies of Targeting Alternative and Lectin Pathway Components in Complement-Mediated Diseases. Front Immunol 9:1851.
Dobó J, Major B, Kékesi K A, Szabó I, Megyeri M, Hajela K, Juhász G, Závodszky P, Gál P. (2011) Cleavage of kininogen and subsequent bradykinin release by the complement component: mannose-binding lectin-associated serine protease (MASP)-1. PLoS One. 6(5):e20036.

Dobó J, Pál G, Cervenak L, Gál P. (2016a) The emerging roles of mannose-binding lectin-associated serine proteases (MASPs) in the lectin pathway of complement and beyond. Immunol Rev. 274(1):98-111.

Dobó J, Szakács D, Oroszlán G, Kortvely E, Kiss B, Boros E, Szász R, Závodszky P, Gál P, Pál G. (2016b) MASP-3 is the exclusive pro-factor D activator in resting blood: the lectin and the alternative complement pathways are fundamentally linked. Sci Rep 6:31877.

Ekdahl K N, Lambris J D, Elwing H, Ricklin D, Nilsson P H, Teramura Y, Nicholls I A, Nilsson B. (2011) Innate immunity activation on biomaterial surfaces: a mechanistic model and coping strategies. Adv Drug Deliv Rev. 63(12):1042-50.

Ekdahl K N, Soveri I, Hilborn J, Fellström B, Nilsson B. (2017) Cardiovascular disease in haemodialysis: role of the intravascular innate immune system. Nat Rev Nephrol. 13(5):285-296.

Fakhouri F, Frémeaux-Bacchi V. Noël L-H, Cook H T, Pickering M C. (2010) C3 glomerulopathy: a new classification. Nat Rev Nephrol (2010) 6:494-9.doi:10.1038/nrneph.2010.85
Empie, M. W. and Laskowski, M., Jr. (1982) Thermodynamics and kinetics of single residue replacements in avian ovomucoid third domains: effect on inhibitor interactions with serine proteinases. Biochemistry 21, 2274-2284.
Fildes, J. E., Shaw, S. M., Walker, A. H., McAlindon, M., Williams, S. G., Keevil, B. G. and Yonan, N. (2008) Mannose-binding lectin deficiency offers protection from acute graft rejection after heart transplantation. J. Heart Lung Transplant. 27, 1353-1356.
Flude B M, Nannetti G, Mitchell P, Compton N, Richards C, Heurich M, Brancale A, Ferla S, Bassetto M. (2021) Targeting the Complement Serine Protease MASP-2 as a Therapeutic Strategy for Coronavirus Infections. Viruses. 13(2):312.
Fritsche L G, Igl W, Bailey J N C, Grassmann F, Sengupta S, Bragg-Gresham J L, et al. (2016) A large genome-wide association study of age-related macular degeneration highlights contributions of rare and common variants. Nat Genet 48:134-43. doi:10.1038/ng.3448
Gál, P., Barna, L., Kocsis, A. and Závodszky P. (2007) Serine proteases of the classical and lectin pathways: Similarities and differences, Immunobiol. 212, 267-277.
Gál P, Dobó J, Závodszky P, Sim R B. (2009) Early complement proteases: C1r, C1s and MASPs. A structural insight into activation and functions. Mol Immunol. 46(14):2745-52.
Gavriilaki E, de Latour R P, Risitano A M. (2022) Advancing therapeutic complement inhibition in hematologic diseases: PNH and beyond. Blood. 139(25):3571-3582.
Geerlings M J, de Jong E K, den Hollander A I. (2017) The complement system in age-related macular degeneration: a review of rare genetic variants and implications for personalized treatment. Mol Immunol 84:65-76. doi:10.1016/j.molimm.2016.11.016
Geisbrecht B V, Lambris J D, Gros P. (2022) Complement component C3: A structural perspective and potential therapeutic implications. Semin Immunol. 24:101627.
Golomingi, M., Kohler, J., Jenny, L., Hardy, E. T., Dobó, J., Gál, P., Pál, P., Kiss, B., Lam, W. A. and Schroeder, V. (2022) Complement lectin pathway components MBL and MASP-1 promote haemostasis upon vessel injury in a microvascular bleeding model. Front. Immunol. doi.org/10.3389/fimmu.2022.948190 Gou, Y., Yang, F., Liang, H. (2016) Designing Prodrugs Based on Special Residues of Human Serum Albumin. Curr. Top. Med. Chem. 16, 996-1008.
Götz M P, Skjoedt M O, Bayarri-Olmos R, Hansen C B, Pérez-Alós L, Jarlhelt I, Benfield T, Rosbjerg A, Garred P. (2022) Lectin Pathway Enzyme MASP-2 and Downstream Complement Activation in COVID-19. J Innate Immun. 11:1-14.
Haddad G, Lorenzen J M, Ma H, de Haan N, Seeger H, Zaghrini C, Brandt S, Kölling M, Wegmann U, Kiss B, Pál G, Gál P, Wuthrich R P, Wuhrer M, Beck L H, Salant D J, Lambeau G, Kistler A D. (2021) Altered glycosylation of IgG4 promotes lectin complement pathway activation in anti-PLA2R1 associated membranous nephropathy. J Clin Invest. 131:e140453.
Hajishengallis G, Reis E S, Mastellos D C, Ricklin D, Lambris J D. (2017) Novel mechanisms and functions of complement. Nat Immunol 18:1288-1298. doi: 10.1038/ni.3858
Harboe M, Ulvund G, Vien L, Fung M, Mollnes T E. (2004) The quantitative role of alternative pathway amplification in classical pathway induced terminal complement activation. Clin Exp Immunol 138:439-446.
Hart M L, Ceonzo K A, Shaffer L A, Takahashi K, Rother R P, Reenstra W R, Buras J A, Stahl G L. (2005) Gastrointestinal ischemia-reperfusion injury is lectin complement pathway dependent without involving C1q. J Immunol. 2005 174(10):6373-80.
Harpaz, Y Gerstein, M and Chothia C. (1994) Volume changes on protein folding. Structure. 2, 641-649.
Héja, D., Kocsis, A., Dobó, J., Szilágyi, K., Szász, R., Závodszky, P., Pál, G. and Gál, P. (2012a) Revised mechanism of complement lectin-pathway activation revealing the role of serine protease MASP-1 as the exclusive activator of MASP-2. Proc. Natl. Acad. Sci. U.S.A 109, 10498-503.
Héja, D., Harmat, V., Fodor, K., Wilmanns, M., Dobó, J., Kékesi, K. A., Závodszky, P., Gál, P., and Pál, G. (2012b) Monospecific inhibitors show that both mannan-binding lectin-associated serine protease-1 (MASP-1) and -2 Are essential for lectin pathway activation and reveal structural plasticity of MASP-2. J. Biol. Chem. 287, 20290-20300.
Hill A, DeZern A E, Kinoshita T, Brodsky R A. Paroxysmal nocturnal haemoglobinuria. (2017) Nat Rev Dis Primer 3:17028. doi:10.1038/nrdp.2017.28
Hofer J, Giner T, Józsi M. (2014) Complement factor H-antibody-associated hemolytic uremic syndrome: pathogenesis, clinical presentation, and treatment. Semin Thromb Hemost 40:431-43. doi:10.1055/s-0034-1375297
Holers V M, Banda N K. (2018) Complement in the Initiation and Evolution of Rheumatoid Arthritis. Front Immunol. 9:1057.
Holmskov U, Thiel S, Jensenius J C. (2003) Collectins and ficolins: humoral lectins of the innate immune defense. Annu Rev Immunol 21:547-578.
Ibernon, M., Moreso, F. and Seron, D. (2014) Innate immunity in renal transplantation: the role of mannose-binding lectin. Transplant. Rev. (Orlando.) 28, 21-25.
Igonin A A, Protsenko D N, Galstyan G M, Vlasenko A V, Khachatryan N N, Nekhaev I V, Shlyapnikov S A, Lazareva N B, Herscu P. (2012) C1-esterase inhibitor infusion increases survival rates for patients with sepsis. Crit Care Med. 40(3):770-7.
Ingram, G., Hakobyan, S., Robertson, N. P. and Morgan, B. P. (2009) Complement in multiple sclerosis: its role in disease and potential as a biomarker. Clin. Exp. Immunol. 155, 128-139.
Jordan J E, Montalto M C, Stahl G L. (2001) Inhibition of mannose-binding lectin reduces postischemic myocardial reperfusion injury. Circulation. 104(12):1413-8.
Keizer, M. P., Pouw, R. B., Kamp, A. M., Patiwael, S., Marsman, G., Hart, M. H., Zeerleder, S., Kuijpers, T. W., Wouters, D. (2015) Eur. J. Immunol. 45, 544-50.
Kidmose, R. T., Laursen, N. S., Dobó, J., Kjaer, T. R., Sirotkina, S., Yatime, L., Sottrup-Jensen, L., Thiel, S., Gál, P. and Andersen, G. R. (2012) Structural basis for activation of the complement system by component C4 cleavage. Proc. Natl. Acad. Sci. U.S.A 109, 15425-30.
Kocsis, A., Kékesi, K. A., Szász, R., Végh, B. M., Balczer, J., Dobó, J., Závodszky, P., Gál, P. and Pal, G. (2010) Selective Inhibition of the Lectin Pathway of Complement with Phage Display Selected Peptides against Mannose-Binding Lectin-Associated Serine Protease (MASP)-1 and -2: Significant Contribution of MASP-1 to Lectin Pathway Activation. J. Immunol. 185, 4169-4178.
La Bonte L R, Dokken B, Davis-Gorman G, Stahl G L, McDonagh P F. (2009) The mannose-binding lectin pathway is a significant contributor to reperfusion injury in the type 2 diabetic heart. Diab Vasc Dis Res. 6(3):172-80.
Lewis, L. A., Ram, S. (2014) Meningococcal disease and the complement system. Virulence. 5, 98-126.
Liu D, Lu F, Qin G, Fernandes S M, Li J, Davis A E 3rd. (2007) C1 inhibitor-mediated protection from sepsis. J Immunol. 179(6):3966-72.
Lobstein, J., Emrich, C. A., Jeans, C., Faulkner, M., Riggs, P., and Berkmen, M. (2012) SHuffle, a novel Escherichia coli protein expression strain capable of correctly folding disulfide bonded proteins in its cytoplasm. Microb. Cell Factories 11, 56.
Magro C, Mulvey J J, Berlin D, Nuovo G, Salvatore S, Harp J, Baxter-Stoltzfus A, Laurence J. (2020) Complement associated microvascular injury and thrombosis in the pathogenesis of severe COVID-19 infection: A report of five cases. Transl Res. 220:1-13.
Mannes M, Dopler A, Zolk O, Lang S J, Halbgebauer R, Höchsmann B, Skerra A, Braun C K, Huber-Lang M, Schrezenmeier H, et al. (2021) Complement inhibition at the level of C3 or C5: mechanistic reasons for ongoing terminal pathway activity. Blood 137:443-455.
Markiewski, M. M. and Lambris, J. D. (2007) The role of complement in inflammatory diseases—from behind the scenes into the spotlight. Am. J. Pathol. 171, 715-727.
Mastellos D C, Ricklin D, Lambris J D. (2019) Clinical promise of next-generation complement therapeutics. Nat Rev Drug Discov. 18(9):707-729.
Mayilyan K R, Arnold J N, Presanis J S, Soghoyan A F, Sim R B. (2006) Increased complement classical and mannan-binding lectin pathway activities in schizophrenia. Neurosci Lett. 404(3):336-41.
McMullen M E, Hart M L, Walsh M C, Buras J, Takahashi K, Stahl G L. (2006) Mannose-binding lectin binds IgM to activate the lectin complement pathway in vitro and in vivo. Immunobiology. 211(10):759-66.
Megyeri M, Harmat V, Major B, Végh Á, Balczer J, Héja D, Szilágyi K, Datz D, Pál G, Závodszky P, et al. (2013) Quantitative characterization of the activation steps of mannan-binding lectin (MBL)-associated serine proteases (MASPs) points to the central role of MASP-1 in the initiation of the complement lectin pathway. J Biol Chem 288:8922-8934.
Meredith, M. E., Salameh, T. S., Banks, W. A. (2015) Intranasal Delivery of Proteins and Peptides in the Treatment of Neurodegenerative Diseases. AAPS. J. 17, 780-787.
Merle, N. S., Church, S. E., Fremeaux-Bacchi, V., Roumenina, L. T. (2015a) Complement System Part I—Molecular Mechanisms of Activation and Regulation. Front. Immunol. 6, 262.
Merle, N. S., Noe, R., Halbwachs-Mecarelli, L., Fremeaux-Bacchi, V. and Roumenina, L. T. (2015b) Complement System Part II: Role in Immunity. Front. Immunol. 6, 257.
Mohammed, B. M., Matafonov A, Ivanov, I., Sun, M., Cheng, Q., Dickeson, S. K., Li, C., Sun, D., Verhamme, I. M., Emsley, J. and Gailani, D. (2018) Thromb. Res. 161, 94-105.
Nauser, C. L., Howard, M. C., Fanelli, G., Farrar, C. A. & Sacks, S. (2018) Collectin-11 (CL-11) is a major sentinel at epithelial surfaces and key pattern recognition molecule in complement-mediated ischaemic injury. Front. Immunol. 9, 2023.
Neumann H P H, Salzmann M, Bohnert-Iwan B, Mannuelian T, Skerka C, Lenk D, et al. (2003) Haemolytic uraemic syndrome and mutations of the factor H gene: a registry-based study of German speaking countries. J Med Genet 40:676-81. doi:10.1136/jmg.40.9.676
Niederreiter J, Eck C, Ries T, Hartmann A, Markl B, Btittner-Herold M, Amann K, Daniel C. (2022) Complement Activation via the Lectin and Alternative Pathway in Patients With Severe COVID-19. Front Immunol. 13:835156.
Noris, M. and Remuzzi, G. (2009) Atypical hemolytic-uremic syndrome. N. Engl. J. Med. 361, 1676-1687.
Oroszlán G, Dani R, Szilágyi A, Závodszky P, Thiel S, Gál P, Dobó J. (2017) Extensive Basal Level Activation of Complement Mannose-Binding Lectin-Associated Serine Protease-3: Kinetic Modeling of Lectin Pathway Activation Provides Possible Mechanism. Front Immunol. 8:1821.
Oroszlán G, Dani R, Végh B M, Varga D, Ács A V, Pál G, Závodszky P, Farkas H, Gál P, Dobó J. (2021) Proprotein Convertase Is the Highest-Level Activator of the Alternative Complement Pathway in the Blood. J Immunol. 206(9):2198-2205.
Orsini, F., Chrysanthou, E., Dudler, T., Cummings, W. J., Takahashi, M., Fujita, T., Demopulos, G., De Simoni, M. G. and Schwaeble, W. (2016) Mannan binding lectin-associated serine protease-2 (MASP-2) critically contributes to post-ischemic brain injury independent of MASP-1. J. Neuroinflammation 13, 213.
Osthoff M, Katan M, Fluri F, Schuetz P. Bingisser R, Kappos L, Steck A J, Engelter S T, Mueller B, Christ-Crain M, Trendelenburg M. (2011) Mannose-binding lectin deficiency is associated with smaller infarction size and favorable outcome in ischemic stroke patients. PLoS One. 2011; 6(6):e21338.
Ozaki, M., Kang, Y., Tan, Y. S., Pavlov, V. I., Liu, B., Boyle, D. C., Kushak, R. I., Skjoedt, M. O., Grabowski, E. F., Taira, Y. and Stahl, G. L. (2016) Human mannose-binding lectin inhibitor prevents Shiga toxin-induced renal injury. Kidney Int. 90, 774-782.
Pangburn M K, Schreiber R D, Müller-Eberhard H J. (1981) Formation of the initial C3 convertase of the alternative complement pathway. Acquisition of C3b-like activities by spontaneous hydrolysis of the putative thioester in native C3. J Exp Med 154:856-867.
Paréj, K., Dobó, J., Závodszky, P. and Gál, P. (2013) The control of the complement lectin pathway activation revisited: both C1-inhibitor and antithrombin are likely physiological inhibitors, while α2-macroglobulin is not. Mol. Immunol. 54, 415-422.
Pavlov, V. I., Skjoedt, M. O., Siow, Tan, Y., Rosbjerg, A., Garred, P. and Stahl, G. L. (2012) Endogenous and natural complement inhibitor attenuates myocardial injury and arterial thrombogenesis. Circulation. 126, 2227-2235.
Pavlov, V. I., Tan, Y. S., McClure, E. E., La Bonte, L. R., Zou, C., Gorsuch, W. B. and Stahl, G. L. (2015) Human mannose-binding lectin inhibitor prevents myocardial injury and arterial thrombogenesis in a novel animal model. Am. J. Pathol. 185, 347-355.
Petersen, B. H., Lee, T. J., Snyderman, R., Brooks, G. F. (1979) Neisseria meningitidis and Neisseria gonorrhoeae bacteremia associated with C6, C7, or C8 deficiency. Ann. Intern. Med. 90, 917-920.
Petri, C., Thiel, S., Jensenius, J. C. and Herlin, T. (2015) Investigation of Complement-activating Pattern Recognition Molecules and Associated Enzymes as Possible Inflammatory Markers in Oligoarticular and Systemic Juvenile Idiopathic Arthritis. J. Rheumatol. 42, 1252-1258.
Pickering M C, D'Agati V D, Nester C M, Smith R J, Haas M, Appel G B, et al. (2013) C3 glomerulopathy: consensus report. Kidney Int 84:1079-89. doi:10.1038/ki.2013.377
Presumey J, Bialas A R, Carroll M C. (2017) Complement system in neural synapse elimination in development and disease. Adv Immunol. 135:53-79. doi:10.1016/bs.ai.2017.06.004
Rambaldi A, Gritti G, Mico M C, Frigeni M, Borleri G, Salvi A, Landi F, Pavoni C, Sonzogni A, Gianatti A, Binda F, Fagiuoli S, Di Marco F, Lorini L, Remuzzi G, Whitaker S, Demopulos G. (2020) Endothelial injury and thrombotic microangiopathy in COVID-19: Treatment with the lectin-pathway inhibitor narsoplimab. Immunobiology. 225(6):152001.
Ricklin D, Mastellos D C, Lambris J D. (2019) Therapeutic targeting of the complement system. Nat Rev Drug Discov. 2019 December 9:10.1038/s41573-019-0055-y.
Ricklin D, Reis E S, Mastellos D C, Gros P, Lambris J D. (2016) Complement component C3—The “Swiss Army Knife” of innate immunity and host defense. Immunol Rev. 274:33-58.
Schechter, I. and Berger, A. (1967) On the size of the active site in proteases. I. Papain. Biochem. Biophys. Res. Commun. 27, 157-162.
Schwaeble, W. J., Lynch, N.J., Clark, J. E., Marber, M., Samani, N.J., Ali, Y. M., Dudler, T., Parent, B., Lhotta, K., Wallis, R., Farrar, C. A., Sacks, S., Lee, H., Zhang, M., Iwaki, D., Takahashi, M., Fujita, T., Tedford, C. E. and Stover, C. M. (2011) Targeting of mannan-binding lectin-associated serine protease-2 confers protection from myocardial and gastrointestinal ischemia/reperfusion injury. Proc. Natl. Acad. Sci. U.S.A 108, 7523-7528.
Sharp T H, Boyle A L, Diebolder C A, Kros A, Koster A J, Gros P. (2019) Insights into IgM-mediated complement activation based on in situ structures of IgM-C1-C4b. Proc Natl Acad Sci USA 116:11900-11905.
Sim R B, Tsiftsoglou S A. (2004) Proteases of the complement system. Biochem Soc Trans 32:21-27. doi: 10.1042/bst0320021 Smith, G. P. (1985) Filamentous Fusion Phage—Novel Expression Vectors That Display Cloned Antigens on the Virion Surface. Science 228, 1315-1317.
Stover C M, Thiel S, Thelen M, Lynch N J, Vorup-Jensen T, Jensenius J C, Schwaeble W J. (1999) Two constituents of the initiation complex of the mannan-binding lectin activation pathway of complement are encoded by a single structural gene. J Immunol. 162(6):3481-90.
Szakács D, Kocsis A, Szász R, Gál P, Pál G. (2019) Novel MASP-2 inhibitors developed via directed evolution of human TFPI1 are potent lectin pathway inhibitors. J. Biol. Chem. 294:8227-8237.
Szebeni, J. (2005) Complement activation-related pseudoallergy: a new class of drug-induced acute immune toxicity. Toxicology 216, 106-121.
Tegla C A, Cudrici C, Patel S, Trippe R, Rus V, Niculescu F, Rus H. (2011) Membrane attack by complement: the assembly and biology of terminal complement complexes. Immunol Res 51:45-60.
Thielens N M, Tedesco F, Bohlson S S, Gaboriaud C, Tenner A J. (2017) C1q: A fresh look upon an old molecule. Mol Immunol. 89:73-83.
Tichaczek-Goska, D. (2012) Deficiencies and excessive human complement system activation in disorders of multifarious etiology. Adv. Clin. Exp. Med. 21, 105-114.
Tobin, P. H., Richards, D. H., Callender, R. A., Wilson, C. J. (2014) Protein engineering: a new frontier for biological therapeutics. Curr. Drug Metab. 15, 743-756.
van den Berg, S., Löfdahl, P. A., Hard, T. and Berglund, H. (2006) Improved solubility of TEV protease by directed evolution. Journal of Biotechnology 121, 291-298.
van Erp I A M, van Essen T A, Fluiter K, van Zwet E, van Vliet P, Baas F, Haitsma I, Verbaan D, Coert B, de Ruiter G C W, Moojen W A, van der Jagt M, Peul W C. (2021) Safety and efficacy of C1-inhibitor in traumatic brain injury (CIAO@TBI): study protocol for a randomized, placebo-controlled, multi-center trial. Trials. 22(1):874.
Zhang M, Takahashi K, Alicot E M, Vorup-Jensen T, Kessler B, Thiel S, Jensenius J C, Ezekowitz R A, Moore F D, Carroll M C. (2006) Activation of the lectin pathway by natural IgM in a model of ischemia/reperfusion injury. J Immunol. 177(7):4727-34.
Zwarthoff S A, Widmer K, Kuipers A, Strasser J, Ruyken M, Aerts P C, de Haas C J C, Ugurlar D, den Boer M A, Vidarsson G, et al. (2021) C1q binding to surface-bound IgG is stabilized by C1r2s2 proteases. Proc Natl Acad Sci USA 118:e2102787118.

1. Protein comprising an amino acid sequence of SEQ ID NO: 115, where the variable positions in the amino acid sequence of SEQ ID NO: 115 are limited in such a way that
x1 to x4, x58, x57, and x56 may be variable or absent,
x6 to x11, x13, x15 to x20, x24 to x29, x31 to x32, x34, x39, x41 to x42, x44, x46 to x50, x52 to x54 may be variable,
x21 is F, Y, or W,
x22 is Y or F,
x23 is Y or F,
x35 is Y or W,
x36 is G or S,
x40 is G or A,
x43 is N or G, and
x45 is F or Y;
characterized in that said protein
i) has an amino acid sequence segment of general formula Ih-mod, starting at position 12 and ending at position 19 of SEQ ID NO: 115:
GX₁CX_{1V}X₂X₃X₄X₅, (Ih-mod)
where
X1 is any of F, Y, L, P, Q, M, V, W, A, or T,
X_{1V} is R, or K,
X₂ is any of A, G, S, or T,
X₃ is any amino acid of the 17-set, where the 17-set comprises A, I, L, F, or Y,
X₄ is any of K, I, Q, R, H, S, F, M, N, L, or V, and
X₅ is any ofR, V, I, K, M, Q, E, F, L, N, Y, D, S, H;
and
ii) in position 34 of the amino acid sequence of SEQ ID NO: 115 it contains any amino acid of the 34-set, where the 34-set comprises Y, I, F, G, V and S;
and salts, esters and pharmaceutically acceptable prodrugs of said protein.

2. Protein, salts, esters and pharmaceutically acceptable prodrugs of said protein according to claim 1, characterized in that said protein is a human MASP-2 inhibitor with a K₁ value equal to or lower than 100 nM.

3. Protein, salts, esters and pharmaceutically acceptable prodrugs of said protein according to claim 1, characterized in that said protein comprises an amino acid sequence, where
i) said amino acid sequence has at least 70%, or at least 80%, or at least 90%, or at least 95% similarity, more preferably at least 98% similarity, even more preferably at least 70%, or at least 80%, or at least 90%, or at least 95% identity, most preferably 98% identity with the amino acid sequence set forth in SEQ ID NO: 116,
with the proviso that
i) said amino acid segment starting at position 12 and ending at position 19 has the sequence defined by the general formula Ih-mod; and
ii) in position 34 of the amino acid sequence of SEQ ID NO: 115 it contains an amino acid selected from the 34-set.

4. Protein, salts, esters and pharmaceutically acceptable prodrugs of said protein according to claim 1, characterized in that said protein comprises an amino acid sequence, where the amino acid pair from the 17-set and 34-set is selected from the group consisting of (in x17/x34 format): A/Y, A/I, A/F, A/G, A/V, A/S, I/Y, I/I, I/F, I/G, I/V, I/S, L/Y, LUI, L/F, L/G, L/V, L/S, F/Y, F/I, F/F, F/G, F/V, F/S, Y/Y, Y/I, Y/F, Y/G, Y/V, Y/S.

5. Protein, salts, esters and pharmaceutically acceptable prodrugs of said protein according to claim 4, characterized in that said amino acid pair from the 17-set and 34-set is selected from the group consisting of (in x17/x34 format): A/Y, A/I, A/F, A/V, I/Y, I/I, I/F, I/G, I/V, I/S, L/Y, L/I, L/F, L/G, L/V, L/S, F/I, F/G, F/V, F/S, Y/Y, Y/I, Y/G, Y/V, Y/S.

6. Protein, salts, esters and pharmaceutically acceptable prodrugs of said protein according to claim 1, characterized in that said protein comprises an amino acid sequence, where in position 9 of the amino acid sequence of SEQ ID NO: 115 said protein contains any amino acid of the 9-set, where the 9-set consists of N or E.

7. Protein, salts, esters and pharmaceutically acceptable prodrugs of said protein according to claim 1, characterized in that said protein comprises an amino acid sequence, where in position 39 of the amino acid sequence of SEQ ID NO: 115 said protein contains any amino acid of the 39-set, where the 39-set consists of F or L.

8. Protein, salts, esters and pharmaceutically acceptable prodrugs of said protein according to claim 1, characterized in that said protein comprises an amino acid sequence, where in position 46 of the amino acid sequence of SEQ ID NO: 115 said protein contains any amino acid of the 46-set, where the 46-set consists of V or E.

9. Protein, salts, esters and pharmaceutically acceptable prodrugs of said protein according to claim 1, characterized in that said protein is selected from proteins comprising an amino acid sequence selected from the group consisting of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 54, SEQ ID NO: 55, and SEQ ID NO: 56.

10. Protein, salts, esters and pharmaceutically acceptable prodrugs of said protein according to claim 1, characterized in that said protein comprises an amino acid sequence that has at least 70%, or at least 80%, or at least 90%, or at least 95% similarity, more preferably at least 98% similarity, even more preferably at least 70%, or at least 80%, or at least 90%, or at least 95% identity, most preferably 98% identity, or is fully identical with any of the amino acid sequences set forth from SEQ ID NO: 3 to SEQ ID NO: 22 and from SEQ ID NO: 24 to SEQ ID NO: 32, with the proviso that the amino acid segment starting at position 12 and ending at position 19 has the sequence defined by the general formula Ih-mod, and in position 34 of the amino acid sequence of SEQ ID NO: 115 it contains an amino acid selected from the 34-set.

11. Protein, salts, esters and pharmaceutically acceptable prodrugs of said protein according to claim 10, characterized in that said protein comprises an amino acid sequence that has at least 95% similarity, more preferably at least 98% similarity, even more preferably 95% identity, most preferably 98% identity, or is fully identical with any of the amino acid sequences set forth in SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 24, SEQ ID NO: 25, and SEQ ID NO: 26, with the proviso that the amino acid segment starting at position 12 and ending at position 19 has the sequence defined by the general formula Ih-mod, and in position 34 of the amino acid sequence of SEQ ID NO: 115 it contains an amino acid selected from the 34-set.

12. Protein, salts, esters and pharmaceutically acceptable prodrugs of said protein according to claim 1, characterized in that said protein is in the form of a fusion protein, which comprises
i) an amino acid sequence of SEQ ID NO: 115, where the variable positions in the amino acid sequence of SEQ ID NO: 115 are limited in such a way that
x1 to x4, x58, x57, and x56 may be variable or absent,
x6 to x11, x13, x15 to x20, x24 to x29, x31 to x32, x34, x39, x41 to x42, x44, x46 to x50, x52 to x54 may be variable,
x 21 is F, Y, or W,
x22 is Y or F,
x23 is Y or F,
x35 is Y or W,
x36 is G or S,
x40 is G or A,
x43 is N or G, and
x45 is F or Y;
within which
a) there is an amino acid sequence segment of general formula Ih-mod, starting at position 12 and ending at position 19 of said SEQ ID NO: 115:
GX₁CX_{1V}X₂X₃X₄X₅, (Ih-mod)
where
X₁ is any of F, Y, L, P, Q, M, V, W, A, or T,
X_{1V} is R, or K,
X₂ is any of A, G, S, or T,
X₃ is any amino acid of the 17-set, where the 17-set comprises A, I, L, F, or Y,
X₄ is any of K, I, Q, R, H, S, F, M, N, L, or V, and
X₅ is any of R, V, I, K, M, Q, E, F, L, N, Y, D, S, H;
and
b) in position 34 of the amino acid sequence of SEQ ID NO: 115 it contains an amino acid selected from the 34-set, where the 34-set comprises Y, I, F, G, V and S; and
ii) an antibody Fc-domain, preferably a human antibody Fc-domain.

13. Protein, salts, esters and pharmaceutically acceptable prodrugs of said protein according to claim 12, characterized in that said fusion protein comprises an amino acid sequence that has at least 70%, or at least 80%, or at least 90%, or at least 95% similarity, more preferably at least 98% similarity, even more preferably at least 70%, or at least 80%, or at least 90%, or at least 95% identity, most preferably 98% identity, or is fully identical with SEQ ID NO: 114.

14. Pharmaceutical preparation, characterized in that it contains at least one protein, its pharmaceutically acceptable salt, pharmaceutically acceptable ester or pharmaceutically acceptable prodrug of claim 1, and at least one additive, said at least one protein is preferably selected from proteins defined by any of the amino acid sequences of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 56; more preferably said at least one protein is selected from the proteins comprising any of the amino acid sequences of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 24, SEQ ID NO: 25, and SEQ ID NO: 26, SEQ ID NO: 27, SEQ ID NO: 28, SEQ ID NO: 29, SEQ ID NO: 30, SEQ ID NO: 31, and SEQ ID NO: 32; most preferably said at least one protein is selected from the proteins comprising any of the amino acid sequences of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 24, SEQ ID NO: 25, and SEQ ID NO: 26.

15. Pharmaceutical preparation according to claim 14, characterized in that said additive is preferably a matrix ensuring controlled active agent release.

16. Pharmaceutical preparation according to claim 14, characterized in that said pharmaceutical preparation is in the form of infusions, tablets, powders, granules, suppositories, injections, syrups, and inhalation and intranasal delivery systems.

17. Nucleic acid encoding a protein of claim 1.

18. Vector, comprising the nucleic acid of claim 17.

19. Kit containing at least one protein, its salt or ester of claim 1, and manual for use or reference to such manual.

20. Screening procedure of compounds potentially inhibiting a MASP-2 enzyme, preferably the human MASP-2 enzyme, in the course of which i) a protein, its salt or ester, of claim 1, in a labelled form, is added to a solution containing said MASP-2 enzyme, preferably said human MASP-2 enzyme, then ii) the solution containing one or more compounds to be tested is added to it, and iii) the amount of the released labelled protein is measured.

21. A method of inhibiting a MASP-2 protein comprising administering to a subject in need thereof the proteins, their salts, esters or prodrugs, of claim 1.

22. A method of treating or preventing diseases that can be treated by inhibiting the complement system comprising administering a pharmaceutical preparation comprising the proteins, their pharmaceutically acceptable salts, pharmaceutically acceptable esters or pharmaceutically acceptable prodrugs, of claim 1 to a subject in need thereof.

23. The method of claim 22, characterized in that said diseases are selected from the following list: (1) ischemia-reperfusion (IR) injuries (especially following recanalization after arterial occlusion due to thrombosis or other obstructive diseases), including those occurring after myocardial infarction, coronary bypass surgery, IR injury of the graft at organ transplantations, gastrointestinal IR injury, renal IR injury, post-ischemic brain injury, stroke, thrombosis affecting any region of the body; (2) inflammatory and autoimmune conditions with excess activation of the complement system, including autoimmune nephritis (including dense deposit disease, C3 glomerulonephritis), IgA nephropathy, membranous nephropathy, rheumatoid arthritis (RA), juvenile idiopathic arthritis, age-related macular degeneration, systemic lupus erythematosus (SLE), atypical hemolytic uremic syndrome (aHUS), thrombotic microangiopathy (TMA), post-infection hemolytic uremic syndrome (HUS), pseudo-allergy developing as a consequence of complement activation (CARPA), paroxysmal nocturnal hemoglobinuria (PNH), polytrauma, graft rejection after organ transplantation; venous thromboembolism (3) neurodegenerative diseases, preferably Alzheimer's disease, Huntington's disease, Parkinson's disease, multiple sclerosis and age-related macular degeneration; (4) complement overactivation caused by viral infection such as COVID-19 (SARS-CoV-2), acute respiratory distress syndrome (ARDS), complement associated microvascular injury and thrombosis due to severe COVID-19 infection.

24. A process for isolating the human MASP-2 enzyme, comprising i) contacting a carrier with one or more immobilised proteins, their pharmaceutically acceptable salts, esters, of claim 1 with a solution containing said human MASP-2 enzyme and ii) washing the preparation.
